(19) United States
(12) Patent Application Publication
(10) Pub. No.: US 2006/0264384 A1
(43) Pub. Date: Nov. 23, 2006

(54) Compositions and methods for treatment for neoplasms

(76) Inventors: Lisa M. Johansen, Belmont, MA (US); Margaret S. Lee, Middleton, MA (US); M. James Nichols, Boston, MA (US); Grant R. Zimmermann, Somerville, MA (US)

Correspondence Address: CLARK & ELBING LLP, 101 FEDERAL STREET, BOSTON, MA, 02110, US

(21) Appl. No.: 11/429,544

(22) Filed: May 4, 2006

Related U.S. Application Data
(60) Provisional application No. 60/678,078, filed on May 5, 2005.

(51) Int. Cl. A61K 31/7048; A61K 31/522; A61K 31/366; A61K 31/48; A61K 31/4745

(52) U.S. Cl. 514/27; 514/263.32; 514/460; 514/381; 514/283; 514/254.07; 514/411; 514/288

(57) ABSTRACT

The invention features compositions including two, three, or more agents useful in treating a patient with a neoplasm, methods for treatment of a patient with a neoplasm such as cancer (e.g., brain cancer), kits which include one, two, three, or more agents useful in the treatment of cancer, as well as methods for identifying combinations of compounds potentially useful in treating a patient with a neoplasm.

### CROSS-REFERENCE TO RELATED APPLICATIONS

This application claims benefit of U.S. provisional application No. 60/678,078, filed May 5, 2005, which is hereby incorporated by reference.

### BACKGROUND OF THE INVENTION

The invention relates to combinations of drugs and methods for treatment of neoplasms such as cancer (e.g., brain cancer), kits containing compositions and combinations of drugs for the treatment of a neoplasm as well as methods for identifying combinations of compounds useful in treatment of a neoplasm.

Cancer is a disease marked by the uncontrolled growth of abnormal cells. Cancer cells have overcome the barriers imposed in normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream may ensue, destroying healthy tissue.

Brain tumors are the leading cause of death in childhood cancers and the second most common cancer-related cause of death in middle aged males. In 2002, an estimated 17,000 patients in the United States were diagnosed with a primary malignant brain tumor. That same year, 170,000 patients in the United States were diagnosed with a secondary metastatic brain tumor.

Primary brain tumors have an extremely poor prognosis despite aggressive treatment with current therapies. In 2003, the Central Brain Tumor Registry of the United States reported that only 8.2% of patients survived 2 years after diagnosis of the most common primary malignant brain tumor, glioblastoma multiforme, and only 2.9% of these patients survived 5 years.

Thus, a significant unmet need exists for new therapies to treat this disease.

### SUMMARY OF THE INVENTION

The present invention features compositions, methods, and kits useful in the treatment or prevention of a neoplasm such as cancer (e.g., brain cancer). The invention also provides methods for identification of compositions useful in treating neoplasms.

In a first aspect, the invention features a composition (e.g., a composition formulated for oral, systemic, parenteral, intracranial, or intrathecal administration) including a first agent selected from the agents of Table 1 and Table 2; and a second, different agent selected from the agents of Table 1 and Table 2, where the first agent and the second agent may be present in amounts that, when administered to a patient, are sufficient to inhibit the growth of a neoplasm. The composition may further include one or more additional agents selected from Table 1 or Table 2. In particular embodiments, the composition includes those where the first agent and the second agent are cerivastatin and adefovir dipivoxil; irinotecan and adefovir dipivoxil; lovastatin and adefovir dipivoxil; topotecan and adefovir dipivoxil; disulfiram and auranofin; cerivastatin and candesartan cilexetil; lovastatin and candesartan cilexetil; triflupromazine and carvedilol; efavirenz and cerivastatin; lovastatin and efavirenz; lovastatin and epirubicin; irinotecan and idebenone; lovastatin and idebenone; simvastatin and idebenone; norethynodrel and irinotecan; metergoline and itraconazole; paroxetine and itraconazole; triflupromazine and itraconazole; raloxifene and maprotiline; raloxifene and metergoline; sertraline and metergoline; topotecan and norethynodrel; or itraconazole and chlorprothixene.

TABLE 1

| | | | |
|---|---|---|---|
| Adapalene | Ciclopirox | Ibudilast | Pramoxine (e.g., |
| | | | hydrochloride) |
| Adefovir dipivoxil | Clotrimazole | Idebenone | Prazosin (e.g., |
| | | | hydrochloride) |
| Alosetron (e.g., | Colchicine | Isotretinoin | Prednisolone |
| hydrochloride) | | | |
| Amiodarone (e.g., | Curcumin | Itraconazole | Prochlorperazine maleate |
| hydrochloride) | | | |
| Amlodipine besylate | Deferoxamine mesylate | Lomefloxacin (e.g., | Quinacrine |
| | | hydrochloride) | |
| Amodiaquine | Dexamethasone | Lomerizine | Rescinnamine |
| Auranofin | Dipyridamole | Lovastatin | Rilmenidine |
| Azacitidine | Disulfiram | Maprotiline (e.g., | Riluzole |
| | | hydrochloride) | |
| Azelastine | Docetaxel | Metergoline | Secobarbital sodium |
| Beta-carotene | Ebastine | Methacycline (e.g., | Sertraline (e.g., |
| | | hydrochloride) | hydrochloride) |
| Bortezomib | Efavirenz | Nelfinavir mesylate | Sibutramine |
| Bupivacaine (e.g., | Ergotamine tartrate | Nicardipine (e.g., | Simvastatin |
| hydrochloride) | | hydrochloride) | |
| Candesartan cilexetil | Estradiol (e.g., valerate) | Niclosamide | Sirolimus |
| Cantharidin | Ethinyl estradiol | Nifedipine | Spironolactone |
| Carvedilol | Exemestane | Norethynodrel | Testosterone |
| Celecoxib | Felodipine | Oxymetholone | Thalidomide |
| Cerivastatin sodium | Fluorouracil | Paroxetine (e.g., | Triflupromazine (e.g., |
| | | hydrochloride) | hydrochloride) |
| Chlordiazepoxide (e.g., | Fluspirilene | Parthenolide | Vinorelbine |
| hydrochloride) | | | |
| Chloroquine (e.g., | Furazolidone | Perhexiline | Voriconazole |
| phosphate) | | | |
| Chlorprothixene | Griseofulvin, | Phenoxybenzamine | |
| | microcrystalline | | |
| Chrysin | Hymecromone | Pioglitazone (e.g., | |
| | | hydrochloride) | |

TABLE 2

| | |
|---|---|
| Busulfan | Irinotecan hydrochloride |
| Carmustine | Melphalan |
| Cepharanthine | Oxaliplatin |
| Epirubicin hydrochloride | Raloxifene (e.g., |
| | hydrochloride) |
| Gefitinib | Tamoxifen (e.g., citrate) |
| Gemcitabine hydrochloride | Temozolomide |
| Imatinib mesylate | Topotecan (e.g., |
| | hydrochloride) |

In a second aspect, the invention features a method for treating a patient having neoplasm which includes administration to the patient of an agent selected from the agents of Table 1 (FIG. 1) in an amount effective to treat the patient.

In a third aspect, the invention features method for treating a patient having a neoplasm including administration of a plurality of agents (e.g., cerivastatin and adefovir dipivoxil; irinotecan and adefovir dipivoxil; lovastatin and adefovir dipivoxil; topotecan and adefovir dipivoxil; disulfiram and auranofin; cerivastatin and candesartan cilexetil; lovastatin and candesartan cilexetil; triflupromazine and carvedilol; efavirenz and cerivastatin; lovastatin and efavirenz; lovastatin and epirubicin; irinotecan and idebenone; lovastatin and idebenone; simvastatin and idebenone; norethynodrel and irinotecan; metergoline and itraconazole; paroxetine and itraconazole; triflupromazine and itraconazole; raloxifene and maprotiline; raloxifene and metergoline; sertraline and metergoline; topotecan and norethynodrel; or itraconazole and chlorprothixene; shown in FIG. 2) each selected from any of the agents of Table 1 and Table 2, where the agents are administered within 28 days (e.g., within 10 days, five days, or 24 hours) of each other.

In either of the second or third aspects, the neoplasm may be cancer (e.g., brain cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, and retinoblastoma, lung cancer, squamous cell carcinoma, adenocarcinoma, large cell carcinoma, and colon cancer). In a particular embodiment, the cancer is brain cancer (e.g., glioblastoma, astrocytoma, glioma, meduloblastoma, and oligodendroma, neuroglioma, ependymoma, and meningioma). The methods may be performed in conjunction with administration to the patient of an additional treatment for a neoplasm, where the method and the additional treatment are administered within 6 months (e.g., within 14 days, 5 days, or 24 hours) of each other. The additional treatment may be surgery, radiation therapy, chemotherapy (e.g., Group A antiproliferative agents), immunotherapy, anti-angiogenesis therapy, or gene therapy. The chemotherapy may be selected from one or more Group A antiproliferative agents (e.g., bleomycin, carmustine, cisplatin, daunorubicin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, and rituximab, or any combination of these). The agents of the methods of the second and third aspects of the invention may be administered to the patient by intravenous, intramuscular, inhalation, rectal, or oral administration. In another embodiment, the agents are administered by intracranial or intrathecal administration. The methods may further include administration of a compound that increases blood-brain barrier permeability (e.g., a Na⁺/Ca⁺⁺ exchange blocker, mannitol, or Cereport).

The invention also provides for a kit including an agent selected from any one of the agents of Table 1, and instructions for administering the agent to a patient having or at risk of having a neoplasm.

The invention also features a kit including a composition including two agents selected from any one of the agents of Table 1 and Table 2, and instructions for administering the composition to a patient having or at risk of having a neoplasm.

The invention also features a kit including a first agent selected from any one of the agents of Table 1 and Table 2, a second agent selected from any one of the agents of Table 1 and Table 2, and instructions for administering the first and the second agents to a patient having or at risk of having a neoplasm.

The invention also features a kit including (a) an agent selected from any one of the agents of Table 1 and Table 2; and (b) instructions for administering the agent with a second agent selected from any one of the agents of Table 1 and Table 2 to a patient having or at risk of having a neoplasm, wherein the second agent is not the agent in (a).

The invention also features a kit including a composition including a first agent selected from any one of the agents of Table 1 and Table 2, and one or more Group A antiproliferative agents; and instructions for administering the composition to a patient having or at risk of having a neoplasm.

The invention also features a kit including a first agent selected from any one of the agents of Table 1 and Table 2, one or more Group A antiproliferative agents, and instructions for administering both to a patient having or at risk of having a neoplasm.

The invention also features a kit including an agent selected from any one of the agents of Table 1, and instructions for administering the agent and one or more Group A antiproliferative agents.

The invention also features a kit including (a) one or more Group A antiproliferative agents, and (b) instructions for administering the agent from (a) with any agent selected from any one of the agents of Table 1 and Table 2 to a patient having or at risk of having a neoplasm.

The invention also features a method of identifying a combination that may be useful for the treatment, prevention, or reduction of a neoplasm, the method including the steps of contacting neoplastic cells with an agent selected from any one the agents of Table 1 and Table 2 and a candidate compound, and determining whether the combination of the agent and the candidate compound inhibits the growth of a neoplasm relative to cells contacted with the agent but not contacted with the candidate compound, where a reduction in proliferation (e.g., a reduction in proliferation resulting from a decreased rate of cellular division, toxicity to rapidly dividing cells, an increase in apoptotic death, or an increase in necrotic death) identifies the combination as a combination useful for the treatment, prevention, or reduction of a neoplasm. The neoplastic cells may be mammalian cells, for example, human cells (e.g., neuronal cells, glial cells, microglial cells, oligodendrocytes, or astrocytes).

By “Group A antiproliferative agent” is meant an agent listed in Table 3.

TABLE 3

| | | |
|---|---|---|
| Alkylating agents | cyclophosphamide | procarbazine |
| | ifosfamide | altretamine |
| | hexamethylmelamine | estramustine phosphate |
| | thiotepa | mechlorethamine |
| | chlorambucil | streptozocin |
| | dacarbazine | semustine |
| | lomustine | |
| Platinum agents | cisplatin | ZD-0473 (AnorMED) |
| | spiroplatinum | lobaplatin (Aeterna) |
| | carboxyphthalatoplatinum | satraplatin (Johnson Matthey) |
| | tetraplatin | BBR-3464 (Hoffmann-La Roche) |
| | ormiplatin | SM-11355 (Sumitomo) |
| | iproplatin | AP-5280 (Access) |
| | carboplatinum | |
| Antimetabolites | azacytidine | trimetrexate |
| | capecitabine | deoxycoformycin |
| | 5-fluorouracil | fludarabine |
| | floxuridine | pentostatin |
| | 2-chlorodeoxyadenosine | raltitrexed |
| | 6-mercaptopurine | hydroxyurea |
| | 6-thioguanine | decitabine (SuperGen) |
| | cytarabin | clofarabine (Bioenvision) |
| | 2-fluorodeoxy cytidine | irofulven (MGI Pharma) |
| | methotrexate | DMDC (Hoffmann-La Roche) |
| | idatrexate | ethynylcytidine (Taiho) |
| | tomudex | |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | J-107088 (Merck & Co) |
| | rebeccamycin analogue (Exelixis) | BNP-1350 (BioNumerik) |
| | BBR-3576 (Novuspharma) | CKD-602 (Chong Kun Dang) |
| | rubitecan (SuperGen) | KW-2170 (Kyowa Hakko) |
| Antitumor | dactinomycin (actinomycin D) | amonafide |
| antibiotics | doxorubicin (adriamycin) | azonafide |
| | deoxyrubicin | anthrapyrazole |
| | valrubicin | oxantrazole |
| | daunorubicin (daunomycin) | losoxantrone |
| | therarubicin | bleomycin sulfate (blenoxane) |
| | idarubicin | bleomycinic acid |
| | rubidazone | bleomycin A |
| | plicamycinp | bleomycin B |
| | porfiromycin | mitomycin C |
| | cyanomorpholinodoxorubicin | MEN-10755 (Menarini) |
| | mitoxantrone (novantrone) | GPX-100 (Gem Pharmaceuticals) |
| Antimitotic | paclitaxel | SB 408075 (GlaxoSmithKline) |
| agents | docetaxel | E7010 (Abbott) |
| | colchicine | PG-TXL (Cell Therapeutics) |
| | vinblastine | IDN 5109 (Bayer) |
| | vincristine | A 105972 (Abbott) |
| | vinorelbine | A 204197 (Abbott) |
| | vindesine | LU 223651 (BASF) |
| | dolastatin 10 (NCI) | D 24851 (ASTAMedica) |
| | rhizoxin (Fujisawa) | ER-86526 (Eisai) |
| | mivobulin (Warner-Lambert) | combretastatin A4 (BMS) |
| | cemadotin (BASF) | isohomohalichondrin-B (PharmaMar) |
| | RPR 109881A (Aventis) | ZD 6126 (AstraZeneca) |
| | TXD 258 (Aventis) | PEG-paclitaxel (Enzon) |
| | epothilone B (Novartis) | AZ10992 (Asahi) |
| | T 900607 (Tularik) | IDN-5109 (Indena) |
| | T 138067 (Tularik) | AVLB (Prescient NeuroPharma) |
| | cryptophycin 52 (Eli Lilly) | azaepothilone B (BMS) |
| | vinflunine (Fabre) | BNP-7787 (BioNumerik) |
| | auristatin PE (Teikoku Hormone) | CA-4 prodrug (OXiGENE) |
| | BMS 247550 (BMS) | dolastatin-10 (NIH) |
| | BMS 184476 (BMS) | CA-4 (OXiGENE) |
| | BMS 188797 (BMS) | |
| | taxoprexin (Protarga) | |
| Aromatase | aminoglutethimide | exemestane |
| inhibitors | letrozole | atamestane (BioMedicines) |
| | anastrazole | YM-511 (Yamanouchi) |
| | formestane | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | mafosfamide (Baxter International) |
| | glufosfamide (Baxter International) | apaziquone (Spectrum Pharmaceuticals) |
| | albumin + 32P (Isotope Solutions) | O6 benzyl guanine (Paligent) |
| | thymectacin (NewBiotics) | |
| | edotreotide (Novartis) | |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | methylprednisolone |
| antihormonal | conjugated estrogens | prednisolone |
| agents | ethinyl estradiol | aminoglutethimide |
| | chlortrianisen | leuprolide |
| | idenestrol | goserelin |
| | hydroxyprogesterone caproate | leuporelin |
| | medroxyprogesterone | bicalutamide |
| | testosterone | flutamide |
| | testosterone propionate; fluoxymesterone | octreotide |
| | methyltestosterone | nilutamide |
| | diethylstilbestrol | mitotane |
| | megestrol | P-04 (Novogen) |
| | toremofine | 2-methoxyestradiol (EntreMed) |
| | dexamethasone | arzoxifene (Eli Lilly) |
| | prednisone | |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Tyrosine Kinase | leflunomide (Sugen/Pharmacia) | kahalide F (PharmaMar) |
| Inhibitors | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | phenoxodiol () |
| | SU6668 (Pharmacia) | trastuzumab (Genentech) |
| | ZD4190 (AstraZeneca) | C225 (ImClone) |
| | ZD6474 (AstraZeneca) | rhu-Mab (Genentech) |
| | vatalanib (Novartis) | MDX-H210 (Medarex) |
| | PKI166 (Novartis) | 2C4 (Genentech) |
| | GW2016 (GlaxoSmithKline) | MDX-447 (Medarex) |
| | EKB-509 (Wyeth) | ABX-EGF (Abgenix) |
| | EKB-569 (Wyeth) | IMC-1C11 (ImClone) |

Miscellaneous agents

| | |
|---|---|
| SR-27897 (CCK A inhibitor, Sanofi-Synthelabo) | BCX-1777 (PNP inhibitor, BioCryst) |
| tocladesine (cyclic AMP agonist, Ribapharm) | ranpirnase (ribonuclease stimulant, Alfacell) |
| alvocidib (CDK inhibitor, Aventis) | galarubicin (RNA synthesis inhibitor, Dong-A) |
| CV-247 (COX-2 inhibitor, Ivy Medical) | tirapazamine (reducing agent, SRI International) |
| P54 (COX-2 inhibitor, Phytopharm) | N-acetylcysteine (reducing agent, Zambon) |
| CapCell ™ (CYP450 stimulant, Bavarian Nordic) | R-flurbiprofen (NF-kappaB inhibitor, Encore) |
| GCS-100 (gal3 antagonist, GlycoGenesys) | 3CPA (NF-kappaB inhibitor, Active Biotech) |
| G17DT immunogen (gastrin inhibitor, Aphton) | seocalcitol (vitamin D receptor agonist, Leo) |
| efaproxiral (oxygenator, Allos Therapeutics) | 131-I-TM-601 (DNA antagonist, TransMolecular) |
| PI-88 (heparanase inhibitor, Progen) | eflornithine (ODC inhibitor, ILEX Oncology) |
| tesmilifene (histamine antagonist, YM BioSciences) | minodronic acid (osteoclast inhibitor, Yamanouchi) |
| histamine (histamine H2 receptor agonist, Maxim) | indisulam (p53 stimulant, Eisai) |
| tiazofurin (IMPDH inhibitor, Ribapharm) | aplidine (PPT inhibitor, PharmaMar) |
| cilengitide (integrin antagonist, Merck KGaA) | rituximab (CD20 antibody, Genentech) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promoter, Procyon) |
| bortezomib (proteasome inhibitor, Millennium) | doranidazole (apoptosis promoter, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promoter, MAXIA) |
| midostaurin (PKC inhibitor, Novartis) | apomine (apoptosis promoter, ILEX Oncology) |
| bryostatin-1 (PKC stimulant, GPC Biotech) | urocidin (apoptosis promoter, Bioniche) |
| CDA-II (apoptosis promoter, Everlife) | Ro-31-7453 (apoptosis promoter, La Roche) |
| SDX-101 (apoptosis promoter, Salmedix) | brostallicin (apoptosis promoter, Pharmacia) |
| ceflatonin (apoptosis promoter, ChemGenex) | |

Analogs of any of the compounds listed in Table 1, Table 2, and Table 3 may be used in any of the methods, kits, and compositions of the invention. Such analogs include any agent from the same chemical class, mechanistic class, or therapeutic class as the compounds of Table 1, Table 2, and Table 3, and include those described herein.

Compounds useful in the invention include those described herein (e.g., in Table 1, Table 2, and Table 3) in any of their pharmaceutically acceptable forms, including isomers such as diastereomers and enantiomers, salts, solvates, and polymorphs, thereof, as well as racemic mixtures of the compounds described herein.

By “patient” is meant any animal (e.g., a mammal such as a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

To “treat” is meant to administer one or more agents to measurably slow, stop, or reverse the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “an effective amount” is meant the amount of a compound, alone or in combination with another therapeutic regimen, required to treat a patient with a neoplasm such as cancer (e.g., brain cancer) in a clinically relevant manner. A sufficient amount of an active compound used to practice the present invention for therapeutic treatment of conditions caused by or contributing to a neoplasm varies depending upon the manner of administration, the age, body weight, and general health of the patient. Ultimately, the prescribers will decide the appropriate amount and dosage regimen. Additionally, an effective amount may be an amount of compound in the combination of the invention that is safe and efficacious in the treatment of a patient having a neoplasm such as cancer (e.g., brain cancer) over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components or derivatives thereof.

By “rapidly dividing cells” is meant cells (e.g., neoplastic cells, or blastoma cells) that undergo cellular division a rate that is at least 5%, 10%, 15%, 25%, 50%, 75%, 100%, 150%, 200%, or 500% greater than control cells (e.g., non-neoplastic cells) of the same cell type.

In the generic descriptions of compounds of this invention, the number of atoms of a particular type in a substituent group is generally given as a range, e.g., an alkyl group containing from 1 to 4 carbon atoms or C₁₋₄alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “C₁₋₄ alkyl” is meant a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl- I -propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3, or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” or “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)+, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the following Detailed Description, the drawings, and the claims.

### BRIEF DESCRIPTION OF THE DRAWINGS

FIG. 1 shows structures of compounds screened for anti-proliferative activity in the human D54MG cell line and the results of the screen, which are shown as graphs indicating the relationship between concentration of each compound and percent inhibition of growth of the cells.

FIG. 2 shows pairwise combinations identified that exhibit enhanced anti-proliferative activity when both compounds of the pair are used together. The results from the anti-proliferative assay using a 9×9 matrix of a range of concentrations for each compound are shown; excess inhibition for each pair is shown using the (highest single agent) HSA, Bliss, and ADD models.

### DETAILED DESCRIPTION

We have identified compounds that, alone or in combination, may be effective in the treatment of a patient with a neoplasm such as cancer (e.g., brain cancer). Accordingly, the invention features a composition including two or more compounds identified herein, methods for treating a patient (e.g., a mammal such as a human) that has been diagnosed with or is at risk of having a neoplasm by administering one, two, three, or more agents from Table 1 and/or Table 2, kits containing one, two, three, or more agents from Table 1, Table 2, and/or Table 3, and screening methods for identifying combinations of compounds that may be useful in treating a patient having a neoplasm. Optionally, analogs (e.g., those described herein) of these agents may be employed in the methods and compositions of the invention. In the case of cancer, for example, administration of compound(s) in the treatment methods of the invention may reduce cell proliferation and tumor growth. The ability of the agent to cause the reduction in cell proliferation may be attributed, for example, to its ability to increase the rate of cell death of the cancer cells (e.g., necrotic or apoptotic death) or to decrease the rate of cell division of the cancer cells. Optionally, the patient may also receive other therapeutic regimens (e.g., surgery, radiation therapy, chemotherapy, immunotherapy, anti-angiogenesis therapy, and gene therapy). The compounds or combinations of compounds may enhance the efficacy of the other therapeutic regimens such that the dosage, frequency, or duration of the other therapeutic regimen is lowered to achieve the same therapeutic benefit, thereby moderating any unwanted side effects.

In one particular example, the patient being treated is administered two agents listed in Table 1 and/or Table 2 within 28 days of each other in amounts that together are sufficient to treat a patient having or at risk of having a neoplasm. The two agents are desirably administered within 14 days of each other, more desirably within seven days of each other, and even more desirably within twenty-four hours of each other, or even simultaneously (i.e., concomitantly). If desired, either one of the two agents may be administered in low dosage.

Campthotecin Derivatives

Camptothecin is an alkaloid found in Camptotheca accuminata. It has topoisomerase I inhibitory activity and has been used in the treatment of cancer.

The structure of camptothecin is:

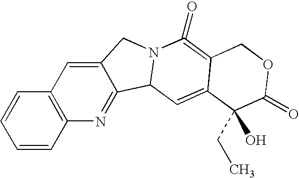

Derivatives of camptothecin are described, for example, in U.S. Pat. No. 3,894,029 and include compounds with the general structure: 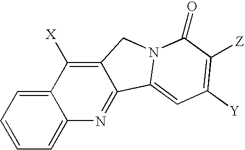
where X is a hydrogen, chlorine, bromine, alkoxy or dialkyl-amino; Y is —CH(COOR)₂; Z is —CH₂OH; or Y and Z together are 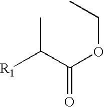
where R is a sterically hindering alkyl and R₁ is a hydrogen or C₁₋₄ alkyl.

Other camptothecin analogs include 9-aminocamptothecin, rubitecan, exatecan, lurtotecan, 7-hydroxymethylcamptothecin, 5-hydroxycamptothecin, 20-O-acetyl-7-acetoxymethylcamptothecin, 7-acetoxymethylcamptothecin, 7-succinoyloxymethylcamptothecin, 20-O-trifluoroacetyl-7-trifluoroacetoxymethylcamptothecin, 7-benzoyloxymethylcamptothecin, 7-propionyloxymethylcamptothecin, 7-butyryloxymethylcamptothecin, 7-caprylyloxymethylcamptothecin, 7-capryloxymethylcamptothecin, 7-isovaleryloxymethylcamptothecin, 7-phenylacetoxymethylcamptothecin, camptothecin-7-carboxylic acid, ethyl camptothecin-7-carboxylate, 5-methoxycamptothecin, 5-butoxycamptothecin, 5-acetoxycamptothecin, 20-O-acetyl-5-acetoxycamptothecin, 5-benzoyloxycamptothecin, 7-methylcamptothecin, 7-ethylcamptothecin, 7-propylcamptothecin, 7-butylcamptothecin, 7-heptylcamptothecin, 7-nonylcamptothecin, 7-isobutylcamptothecin, 7-benzylcamptothecin, 7-.beta.-phenethylcamptothecin, 7-isopropylcamptothecin, 7-cyclohexylcamptothecin, 1-allyl-1-hydroxy-1,2,5,7-tetrahydro-4H-pyrano[3,4-f]indolizino[1,2-b]-quinoline-2,5-dione, 1-hydroxy-1-propargyl-1,2,5,7-tetrahydro-4H-pyrano[3,4-f]indolizino[1,2-b]-quinoline-2,5dione, 1-benzyl-1-hydroxy-1,2,5,7-tetrahydro-4H-pyrano[3,4-f]indolizino[1,2-b]-quinoline-2,5-dione, and the camptothecin analogs described in U.S. Pat. Nos. 4,031,098, 4,399,282, 4,604,463, RE32,518, 4,851,399, 4,900,737, 4,943,579, 5,122,606, 5,180,722, 5,401,747, 5,446,047, 5,468,754, 5,525,731, 5,527,913, 5,541,327, 5,646,159, 5,658,920, 5,663,260, 5,731,316, 5,801,167, 5,889,017, 5,910,491, 5,916,896, 5,968,943, 5,972,955, 6,040,313, 6,096,336, 6,100,273, 6,214,836, 6,218,399, 6,228,855, 6,352,996, 6,407,118, 6,407,239, and 6,706,734. Particularly useful derivatives include irinotecan and topotecan.

Irinotecan

Irinotecan is currently used for treatment of cancer, and its mechanism of action is inhibition of topoisomerase I activity. The structure of irinotecan is: 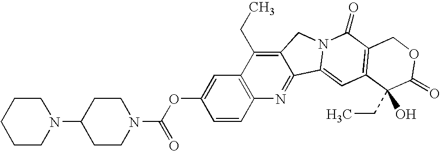
Analogs of irinotecan are described, for example, in U.S. Pat. No. 4,604,463 and have the general structure: 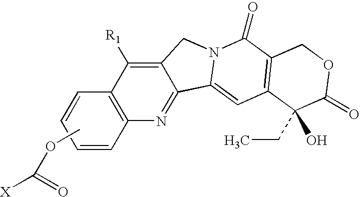
where R₁ is a hydrogen atom, a halogen atom, or a C₁₋₄ alkyl, and X is a chlorine or —NR₂R₃, wherein R₂ and R₃ are the same or different and each represents a hydrogen atom, a C₁₋₄ alkyl, or a substituted or unsubstituted carbocyclic or heterocyclic group, with the proviso that when both R₂ and R₃ are the substituted or unsubstituted alkyl groups, they may be combined together with the nitrogen atom, to which they are bonded, to form a heterocyclic ring which may be interrupted with —O—, —S—, and/or >N—R₄ in which R₄ is a hydrogen atom, a substituted or unsubstituted C₁₋₄ alkyl, or a substituted or unsubstituted phenyl group and where the grouping —O—CO—X is bonded to a carbon atom located in any of the 9-, 10-, and 11-positions in the ring A of camptothecin.

Irinotecan is available as for delivery by intravenous injection, supplied as an aqueous solution. It is commonly in hydrochloride form, which is a yellow powder slightly soluble in water and organic solvents.

Topotecan

Topotecan, a derivative of campthecin, has topoisomerase I inhibitory activity and is used in the treatment of cancer. The structure of topotecan is:

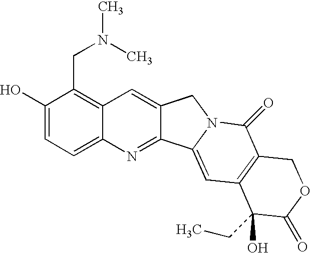

Analogs of topotecan are described, for example, in European Patent 321,122 and include compounds with the general formula: 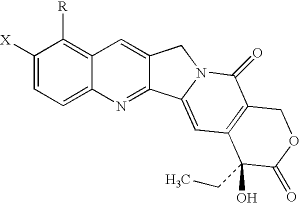
wherein X is hydroxy, hydrogen, cyano, —CH₂NH₂, or formyl; R is hydrogen when X is cyano, CH₂NH₂ or formyl or R is —CHO or —CH₂R₁ when X is hydrogen or hydroxy; R₁ is —O—R₂, —S—R₂, —N—R₂(R₃); or —N⁺—R₂—(R₃)(R₄), R₂, R₃, and R₄ are the same or different and are selected from H, C₁₋₆ alkyl, C₂₋₆ hydroxyalkyl, C₁₋₆ dialkyamino, C₁₋₆-dialkylaminoC₂₋₆alkyl, C₁₋₆ alkyamino-C₂₋₆ alkyl, C₂₋₆ aminoalkyl, or a 3-7 member unsubstituted or substituted carbocyclic ring; and when R₁ is —N—R₂(R₃), the R₂ and R₃ groups may be combined together to form a ring.

Topotecan is light yellowish to green powder and is soluble in water up to 1 mg/ml. The powder is typically reconstituted in solution prior to administration to a patient via intravenous injection.

Adefovir Dipivoxil

Adefovir dipivoxil has antiviral properties and is used in the treatment of HIV and hepatitis B. The structure of adefovir dipivoxil is:

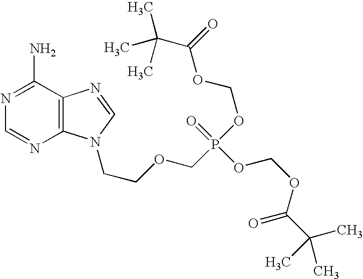

Adefovir dipivoxil is derived from adefovir. Analogs of adefovir are described, for example, in U.S. Pat. No. 4,808,716 and include compounds with the general structure: 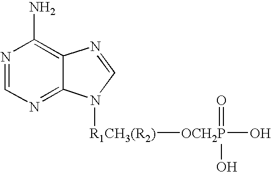
wherein R₁ is a hydrogen atom, an alkyl group containing one to three carbon atoms, or a hydroxymethyl group, and R₂ is a methylene, ethylene, propylene, ethylidene, methoxyethylene, benzyloxyethylene, tetrahydropyran-2-yloxyethylene, (1-ethoxyethoxy)ethylene, or 1,2-O-isopropylidene-1,2-dihydroxypropylene group.
Disulfuram

Disulfiram is used in the treatment of alcoholism; its mechanism of action is inhibition of alcohol dehydrogenase. The structure of disulfiram is:

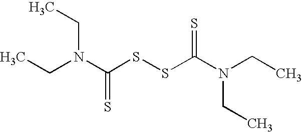

Analogs of disulfiram are described in, for example, U.S. Pat. No. 1,796,977 and have the general structure: 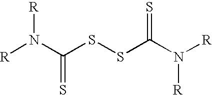
wherein the R groups represent same of dissimilar organic groups (e.g., C₁₋₄ alkyls).

Disulfiram is a crystal, barely soluble in water, and is soluble in solvents such as alcohol, ether, acetone, and benzene. Disulfiram is available in tablet form, and is typically administered orally.

Auranofin

Auranofin is an anti-inflammatory agent and an antirheumatic. The structure of auranofin is:

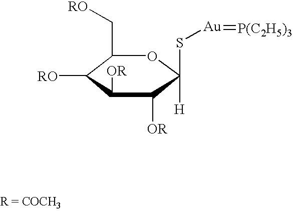

Analogs of auranofin are described, for example, in U.S. Pat. No. 3,635,945, and can be represented by the general formulas: 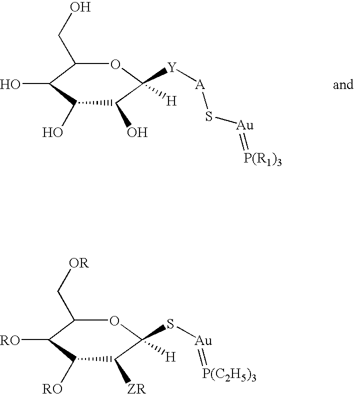
where R represents acetyl or, when Z is oxygen, hydrogen; R₁ represents a C₁₋₄ alkyl; A represents a C₂₋₅ alkylene chain, straight or branched; Y represents oxygen or sulfur; and Z represents oxygen or —NH—.

Auronfin is a white, odorless, crystaline powder and is insoluble in water. It is administered orally in tablet form.

Norethynodrel

Norethynodrel is an orally active estrogenic steroid used as a contraceptive. The structure of norethynodrel is:

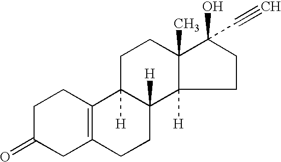

Analogs of norethynodrel are described, for example, in U.S. Pat. No. 2,691,028, and can be represented by: 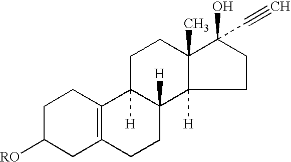
wherein R is a lower alkyl, a lower phenylalkyl (e.g., methyl, ethyl, benzyl, straight and branch chained propyl, butyl, amyl, hexyl, phenethyl, and phenylpropyl, or an ethynyl or vinyl group).

Norethynodrel forms crystals from aqueous methanol.

Analogs

Analogs of any of the compounds listed in Table 1 or Table 2 may be used in any of the compositions, methods, and kits of the invention. Analogs are known in the art (e.g., as described herein). Adapalene analogs are described in European Patent 199,636 and U.S. Pat. No. 4,717,720. Adefovir dipivoxil analogs are described in European Patents 206,459 and 481,214 and U.S. Pat. No. 4,808,716 and 5,663,159. Alosetron hydrochloride analogs are described in European Patent 306,323 and U.S. Pat. No. 5,360,800. Amiodarone analogs are described in French Patent 1,339,389 and U.S. Pat. No. 3,248,401. Amlodipine analogs are described in European Patent 89,167 and U.S. Pat. No. 4,572,909. Amodiaquine analogs are described in U.S. Pat. Nos. 2,474,819 and 2,474,821. Auranofin analogs are described in German Patent 2,051,495 and U.S. Pat. No. 3,635,945. Azelastine analogs are described in Belgian Patent 778,269 and U.S. Pat. No. 3,813,384. Bupivacaine (e.g., hydrochloride salt) analogs are described in U.S. Pat. No. 2,955,111. Busulfan analogs are described in U.S. Pat. No. 2,917,432. Carvedilol analogs are described in German Patent 2,815,926 and U.S. Pat. No. 4,503,067. Celecoxib analogs are described in WO 95/15316 and U.S. Pat. No. 5,466,823. Cerivastatin sodium analogs are described in European Patent 325,130 and U.S. Pat. No. 5,006,530 and U.S. Pat. No. 5,177,080. Chlordiazepoxide (e.g., hydrochloride salt) analogs are described in U.S. Pat. No. 2,893,992. Chloroquine phosphate analogs are described in German Patent 683,692 and U.S. Pat. No. 2,233,970. Chlorprothixene analogs are described in U.S. Pat. No. 3,046,283. Ciclopirox analogs are described in U.S. Pat. No. 3,883,545. Clotrimazole analogs are described in South African Patent 68 05392 and U.S. Pat. No. 3,705,172. Curcumin analogs are described in German Patent 859,145. Deferoxamine (e.g., mesylate) analogs are described in U.S. Pat. No. 3,471,476. Dipyridamole analogs are described in U.S. Pat. No. 3,031,450. Disulfiram analogs are described in U.S. Pat. No. 1,796,977. Docetaxel analogs are described in U.S. Pat. No. 4,814,470. Ebastine analogs are described in European patent 134,124 and U.S. Pat. No. 4,550,116. Efavirenz analogs are described in European patent 582,455 and U.S. Pat. No. 5,519,021. Epirubicin (e.g., hydrochloride salt) analogs are described in German Patent 2,510,866 and U.S. Pat. No. 4,058,519. Estradiol (e.g., valerate) analogs are described in U.S. Pat. No. 2,096,744. Ethinyl estradiol analogs are described in German Patent 702,063, British Patent 516,444, U.S. Pat. No. 2,243,887, U.S. Pat. No. 2,251,939, U.S. Pat. No. 2,265,976, and U.S. Pat. No. 2,267,257. Exemestane analogs are described in German Patent 3,622,841 and U.S. Pat. No. 4,808,616. Felodipine analogs are described in U.S. Pat. No. 4,264,611. Fluorouracil analogs are described in U.S. Pat. Nos. 2,802,005 and 2,885,396. Fluspirilene analogs are described in Belgian Patent 633,914 and U.S. Pat. No. 3,238,216. Furazolidone analogs are described in British Patent 735,136, U.S. Pat. No. 2,742,462, and U.S. Pat. No. 2,927,110. Gemcitabine (e.g., hydrochloride salt) analogs are described in U.S. Pat. No. 4,808,614 and British Patent 2,136,425. Ibudilast analogs are described in German Patent 2,315,801 and U.S. Pat. No. 3,850,941. Idebenone analogs are described in Gernan Patent 2,519,730 and U.S. Pat. No. 4,271,083. Imatinib (e.g., mesylate) analogs are described in European Patent 564,409 and U.S. Pat. No. 5,521,184. Irinotecan hydrochloride analogs are described in Japanese Publication Kokai 95 18,790 and U.S. Pat. No. 4,604,463. Isotretinoin analogs are described in European Patent 111,325 and U.S. Pat. No. 4,556,518. Itraconazole analogs are described in European Patent 6711 and U.S. Pat. No. 4,267,179. Lomefloxacin analogs are described in Gennan Patent 3,433,924 and U.S. Pat. No. 4,528,287. Lomerizine analogs are described in European Patent 159,566 and U.S. Pat. No. 4,663,325. Maprotiline analogs are described in U.S. Pat. No. 3,399,201. Melphalan analogs are described in U.S. Pat. No. 3,032,584. Metergoline analogs are described in U.S. Pat. No. 3,238,211. Methacycline analogs are described in U.S. Pat. No. 2,984,686. Nelfinavir mesylate analogs are described in WO 95/09843 and U.S. Pat. No. 5,484,926. Nicardipine analogs are described in Belgian Patent 811,324 and U.S. Pat. No. 3,985,758. Niclosamide analogs are described in British Patent 824,345, U.S. Pat. No. 3,079,297, and U.S. Pat. No. 3,113,067. Nifedipine analogs are described in South African Patent 68 01482 and U.S. Pat. No. 3,485,847. Norethynodrel analogs are described in U.S. Pat. No. 2,691,028. Oxymetholone analogs are described in German Patent 1,070,632. Paroxetine analogs are described in German Patent 2,404,113, U.S. Pat. No. 3,912,743, and U.S. Pat. No. 4,007,196. Phenoxybenzamine analogs are described in U.S. Pat. No. 2,599,000. Pioglitazone hydrochloride analogs are described in U.S. Pat. No. 4,687,777. Pramoxine analogs are described in U.S. Pat. No. 2,870,151. Prazosin analogs are described in British Patent 1,156,973, U.S. Pat. No. 3,511,836, and Dutch Patent 7,206,067. Prednisolone analogs are described in U.S. Pat. No. 2,837,464 and U.S. Pat. No. 3,134,718. Prochlorperazine (e.g., maleate) analogs are described in British Patent 780,193, French Patent 1,167,627, and U.S. Pat. No. 2,902,484. Quinacrine analogs are described in German Patents 553,072 and 571,499, and U.S. Pat. No. 2,113,357. Raloxifene (e.g., hydrochloride salt) analogs are described in European Patent 62,503 and U.S. Pat. No. 4,418,068. Rilmenidine analogs are described in German Patent 2,362,754 and U.S. Pat. No. 4,102,890. Riluzole analogs are described in European Patent 50,551 and U.S. Pat. No. 4,370,338. Secobarbital (e.g., sodium salt) analogs are described in U.S. Pat. No. 1,954,429. Sertraline (e.g., hydrochloride salt) analogs are described in European Patent 30,081 and U.S. Pat. No. 4,536,518. Simvastatin analogs are described in European Patent 33,538 and U.S. Pat. No. 4,444,784. Spironolactone analogs are described in U.S. Pat. No. 4,444,784. Tamoxifen analogs are described in Belgian Patent 678,807 and U.S. Pat. No. 4,536,516. Temozolomide analogs are described in German patent 3,231,255 and U.S. Pat. No. 5,260,291. Thalidomide analogs are described in British Patent 768,821. Topotecan (e.g., hydrochloride salt) analogs are described in European Patent 321,122. Triflupromazine hydrochloride analogs are described in British Patent 813,861 and U.S. Pat. No. 2,921,069. Vinorelbine analogs are described in U.S. Pat. No. 4,307,100. Voriconazole analogs are described in European Patent 440,372 and U.S. Pat. No. 5,278,175.

Therapy

The combinations of the invention are useful for the treatment of a patient having a neoplasm such as cancer (e.g., brain cancer). Therapy may be performed alone or in conjunction with another therapy (e.g., surgery, radiation therapy, chemotherapy, immunotherapy, anti-angiogenesis therapy, and gene therapy). Additionally, a patient having a greater risk of developing a neoplasm (e.g., one who is genetically predisposed or one who previously had a neoplasm) may receive prophylactic treatment to inhibit or delay neoplasm formation. The duration of the combination therapy depends on the type of disease or disorder being treated, the age and condition of the patient, the stage and type of the patient's disease, and how the patient responds to the treatment. Therapy may be given in on-and-off cycles that include rest periods so that the patient's body has a chance to recovery from any as yet unforeseen side-effects.

Examples of cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenriglioma, schwannoma, glioblastoma meningioma, melanoma, neuroblastoma, or retinoblastoma).

Conjugates

If desired, the drugs used in any of the combinations described herein may be covalently attached to one another to form a conjugate of formula I.
(A)-(L)-(B) (I)

In formula I, (A) is a drug listed on Table 1 or Table 2 covalently tethered via a linker (L) to (B), a Group A antiproliferative, or a second drug listed on Table 1 or Table 2.

Conjugates of the invention can be administered to a subject by any route and for the treatment of any neoplasm described herein.

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker which contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) a-haloacetyl compounds, which show specificity towards amino groups in the absence of reactive thiol groups and are of the type XCH₂CO— (where X═Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁₋₁₀ alkyl, C₂₋₁₀ alkenyl, C₂₋₁₀ alkynyl, C₂₋₆heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (II):
G¹-(Z¹)ₒ-(Y¹)ᵤ-(Z²)ₛ-(R₃₀)-(Z³)ₜ-(Y²)ᵥ-(Z⁴)ₚ-G² (II)

In formula (II), G¹ is a bond between drug (A) and the linker; G² is a bond between the linker and drug (B); Z¹, Z², Z³, and Z⁴ each, independently, is selected from O, S, and NR₃₁; R₃₁ is hydrogen, C₁₋₄alkyl, C₂₋₄ alkenyl, C₂₋₄alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ-(Y²)ᵥ-(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

Formulation of Pharmaceutical Compositions

The administration of each compound of the combination may be by any suitable means that results in a concentration of the compound that, combined with the other component, inhibits the growth of a neoplasm upon reaching the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of the combination in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the combination is formulated with appropriate excipients into a pharmaceutical composition that, upon administration, releases the combination in a controlled manner. Examples include single or multiple unit tablet or capsule compositions, oil solutions, suspensions, emulsions, microcapsules, molecular complexes, microspheres, nanoparticles, patches, and liposomes.

Parenteral Compositions

The pharmaceutical composition may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. Nos. 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the combination in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

The compositions of the invention may be mixed together in the tablet, or may be partitioned. In one example, a first agent is contained on the inside of the tablet, and a second agent is on the outside, such that a substantial portion of the second agent is released prior to the release of the first agent.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

Controlled Release Oral Dosage Forms

Controlled release compositions for oral use may, e.g., be constructed to release the active agent(s) by controlling the dissolution and/or the diffusion of said active combination.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, DL-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed compositions may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the composition with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Formulations and Methods for Delivery of Agents to Neoplasms in the Brain

Treatment of neoplams in the brain (e.g., glioblastoma, astrocytoma, glioma, meduloblastoma, and oligodendroma, neuroglioma, ependymoma, and meningioma) may be hampered by the inability of an active, therapeutic compound to cross the blood-brain barrier (BBB). Strategies to delivery of compounds of the invention to brain neoplasms include strategies to bypass the BBB (e.g., intracranial administration via craniotomy and intrathecal administration), and strategies to cross the BBB (e.g., the use of compounds that increase permeability of the BBB in conjunction with systemic administration of compositions of the invention), and modification of compounds of the invention to increase their permeability or transport across the blood-brain barrier.

Craniotomy, a procedure known in the art, can be used with any composition of the invention for delivery to the brain. In this approach, a opening in made in the patient's cranium, and a compound is delivered via a catheter. This approach can be used to target a compound to a specific area of the brain.

Intrathecal administration provides another means of bypassing the blood brain barrier for drug delivery. Briefly, drugs are administered to the spinal chord, for example, via lumbar puncture or through the use of devices such as pumps. Lumbar puncture is preferable for single or infrequent administration, whereas constant and/or chronic administration may be achieved using any commercially available pump attached to a intraspinal catheter, for example a pump and catheter made by Medtronic (Minneapolis, Minn.).

To allow for delivery across the BBB, compositions of the invention can be administered along with a compound or compounds that induce a transient increase in permeability of the blood-brain barrier. Such compounds include mannitol, Cereport (RMP-7), and KB-R7943, a Na⁺/Ca⁺⁺ exchange blocker.

Compounds of the invention can be modified (e.g., lipidated, acetylated) to increase transport across the blood-brain barrier following systemic administration (e.g., parenteral), by using chemical modifications standard in the art. In one embodiment, compounds of the invention are conjugated to peptide vectors that are transported across the BBB. For example, compounds may be conjugated to a monoclonal antibody to the human insulin receptor as described by Partridge (Jpn. J. Pharmacol. 87:97-103, 2001), thus permitting the compound to be transported across the BBB following systemic administration. Compounds of the invention can be conjugated to such peptide vectors, for example, using biotin-streptavidin technology.

Delivery of Compositions of the Invention

It is not intended that administration of a combination be limited to a single formulation and delivery method for all compounds of a combination. The combination may be administered using separate formulations and/or delivery methods for each compound of the combination using, for example, any of the above-described formulations and methods. In one example, a first agent is delivered orally, and a second agent is delivered intramuscularly.

Dosages

The dosage of each compound or agent of the claimed combinations depends on several factors, including: the administration method, the neoplasm to be treated, the severity of the neoplasm, whether the neoplasm is to be treated or prevented, and the age, weight, and health of the patient to be treated.

The compound or agent in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. An antiproliferative agent of the invention is usually given by the same route of administration that is known to be effective for delivering it as a monotherapy. When used in combination therapy with another agent according to the methods of this invention, the antiproliferative agent is dosed in amounts and frequencies equivalent to or less than those that result in its effective monotherapeutic use.

Additional Applications

If desired, the compounds of the invention may be employed in mechanistic assays to determine whether other combinations, or single agents, are as effective as the combinations of the invention in inhibiting the growth of a neoplasm such as cancer (e.g., brain cancer) using assays generally known in the art, examples of which are described herein. For example, candidate compounds may be tested, alone or in combination (e.g., with an agent that inhibits the growth of a neoplasm, such as those described herein) and applied to neoplastic cells. After a suitable time, growth of these cells is examined. A decrease in growth identifies a candidate compound or combination of agents as an effective agent for inhibiting the growth of a neoplasm.

The agents of the invention are also useful tools in elucidating mechanistic information about the biological pathways involved in neoplastic disorders such as cancer (e.g., brain cancer). Such information can lead to the development of new combinations or single agents for treating, preventing, or reducing neoplasms. Methods known in the art to determine biological pathways can be used to determine the pathway, or network of pathways affected by contacting neoplastic cells (e.g., glioblastoma cells) with the compounds of the invention. Such methods can include, analyzing cellular constituents that are expressed or repressed after contact with the compounds of the invention as compared to untreated, positive or negative control compounds, and/or new single agents and combinations, or analyzing some other activity of the cell such as an enzymatic activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g., using 2D gels, gene expression profiling. Once identified, such compounds can be used in in vivo models (e.g., knockout or transgenic mice) to further validate the tool or develop new agents or strategies to inhibit the growth of a neoplasm.

As indicated above, the methods of this invention may also be used prophylactically, in patients who are an increased risk of developing a neoplasm. Risk factors include, for example, family history, exposure to known carcinogens, previous neoplasms, presence of molecular markers of cancer, age, race, or sex.

Exemplary Candidate Compounds

Peptide Moieties

Peptides, peptide mimetics, and peptide fragments (whether natural, synthetic or chemically modified) are suitable for use in practicing the invention. Exemplary inhibitors include compounds that reduce the amount of target protein or RNA levels (e.g., antisense compounds, dsRNA, ribozymes) and compounds that compete with endogenous mitotic kinesins or protein tyrosine phosphatases for binding partners (e.g., dominant negative proteins or polynucleotides encoding the same).

Antisense Compounds

The biological activity of any protein that increases cellular growth or reduces apoptic or necrotic death can be reduced through the use of an antisense compound directed to RNA encoding the target protein. Antisense compounds that reduce expression of signaling molecules can be identified using standard techniques. For example, accessible regions of the target the mRNA of the target enzyme can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Res. 28:2597-2604, 2000, and Nucleic Acids Res. 31:4989-4994, 2003.

RNA Interference

The biological activity of a signaling molecule can be reduced through the use of RNA interference (RNAi), employing, e.g., a double stranded RNA (dsRNA) or small interfering RNA (siRNA) directed to the signaling molecule in question (see, e.g., Miyamoto et al., Prog. Cell Cycle Res. 5:349-360, 2003; U.S. Patent Application Publication No. 20030157030). Methods for designing such interfering RNAs are known in the art. For example, software for designing interfering RNA is available from Oligoengine (Seattle, Wash.).

Dominant Negative Proteins

One skilled in the art would know how to make dominant negative proteins to the signaling molecules to be targeted. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992).

### EXAMPLE 1

### Antiproliferative Screening Assay

Experimental Procedures

Approved small molecule drugs selected from a drug library were screened in combination for antiproliferative activity against the D54MG glioblastoma multiforme (GBM) cell line. Cell Titer-Blue dye (Promega) was used to measure the metabolic potential of the D54MG cells and can be taken as an indirect measure of the number of viable cells in the well. Cell Titer-Blue dye is a non-fluorescent dye that is reduced, by living cells, to a red fluorescent product that can be easily quantified.

Tumor Cell Culture

The human D54MG cell line (provided by Dr. Darrell Bigner, Duke Univeristy) was grown at 37±0.5° C and 5% CO₂, in Roswell Park Memorial Institute (RPMI)-1640 media supplemented with 10% fetal bovine serum (FBS), 2 mM glutamine, 1% penicillin, and 1% streptomycin.

Test Compounds

Irinotecan hydrochloride was obtained from Abatra Technology Co (Xi'an, China). Intraconazole and sertraline hydrochloride were obtained from Interchem Corporation (Paramus, N.J.). Paroxetine was obtained from LKT Laboratories, Inc (St. Paul, Minn.). Auranofin was obtained through Professional Compounding Centers of America (Houston, Tex.). Topotecan hydrochloride, adefovir dipivoxil, cerivastatin sodium, candesartan cilexetil, simvastatin, idebenone, efavirenz, carvedilol, and epirubicin hydrochloride were obtained from Sequoia Research Products Ltd. (Oxford, UK). Norethynodrel, disulfiram, metergoline, triflupromazine hydrochloride, raloxifene, maprotiline, and prochlorperazine were obtained from Sigma-Aldrich Co. (St. Louis, Mo.). Lovastatin was purchased from US Pharmacopeial Convention, Inc. (Rockville, Md.). Stock solutions (1000×) of each compound were prepared in DMSO and stored at −20° C. Master stock plates of 2-fold serial dilutions of individual compounds were prepared using a Matrix Platemate liquid handling station. Dilutions plates containing test compounds in culture media were generated from these master stock plates. The final concentration of test compounds in the dilution plates was 10× greater than used in the assay. The dilution plates were used immediately and discarded.

Anti-Proliferation Assay

The anti-proliferation assays were performed in 384-well plates. The D54MG cells were liberated from the culture flask using a solution of 0.25% trypsin. Cells were diluted in culture media such that 3000 cells were delivered in 35 μl of media into each assay well. Next, 4.5 μl of 10× stock solutions from the dilution plates were added to each well of cells in assay plates. Assay plates were incubated for 72 hours. Following incubation, 40 μl of 5% Cell Titer-Blue, in culture media, were added to each assay. Cell Titer-Blue metabolism was quantified by the amount of fluorescence intensity 6 hours after addition. Quantification, using a Wallac Victor V, was taken at the top of the well with stabilized energy lamp control, 100 msec read time, an excitation filter at 530 nm, and an emission filter at 590 nm.

The percent inhibition (% I) for each well was calculated using the following formula:
% I=[(avg. untreated wells−treated well)/(avg. untreated wells)]×100

The average untreated well value (avg. untreated wells) is the arithmetic mean of 31 wells from the same assay plate treated with vehicle alone. The data shown are the average of at least four 9×9 matrices except for the combinations of itraconazole with TCA and metergoline with raloxifene which are the average of two matrices.

Screening

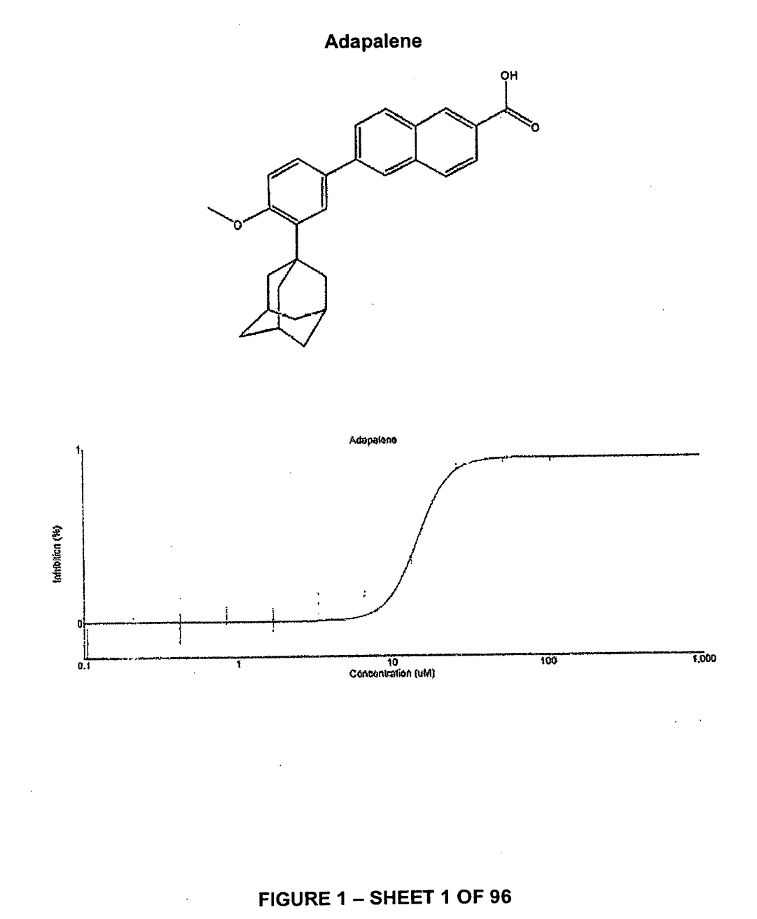

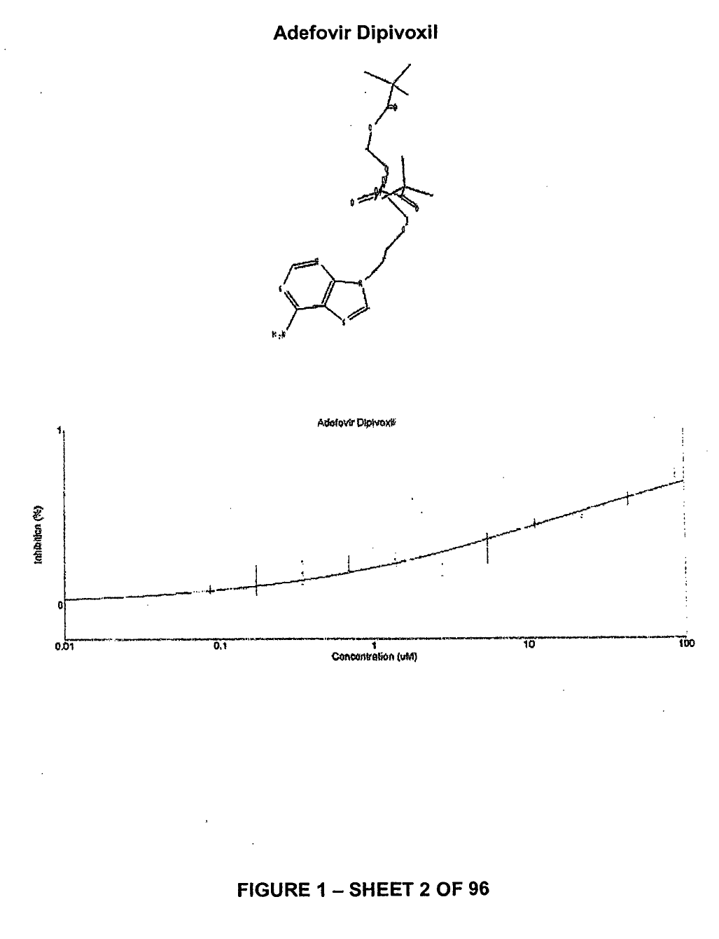

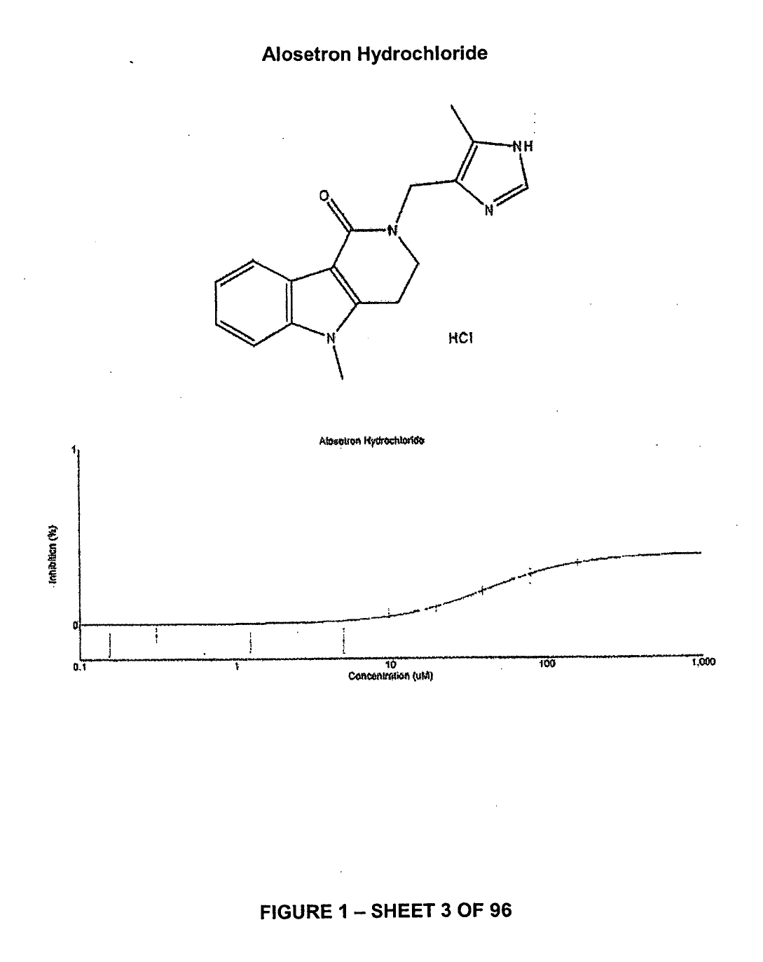

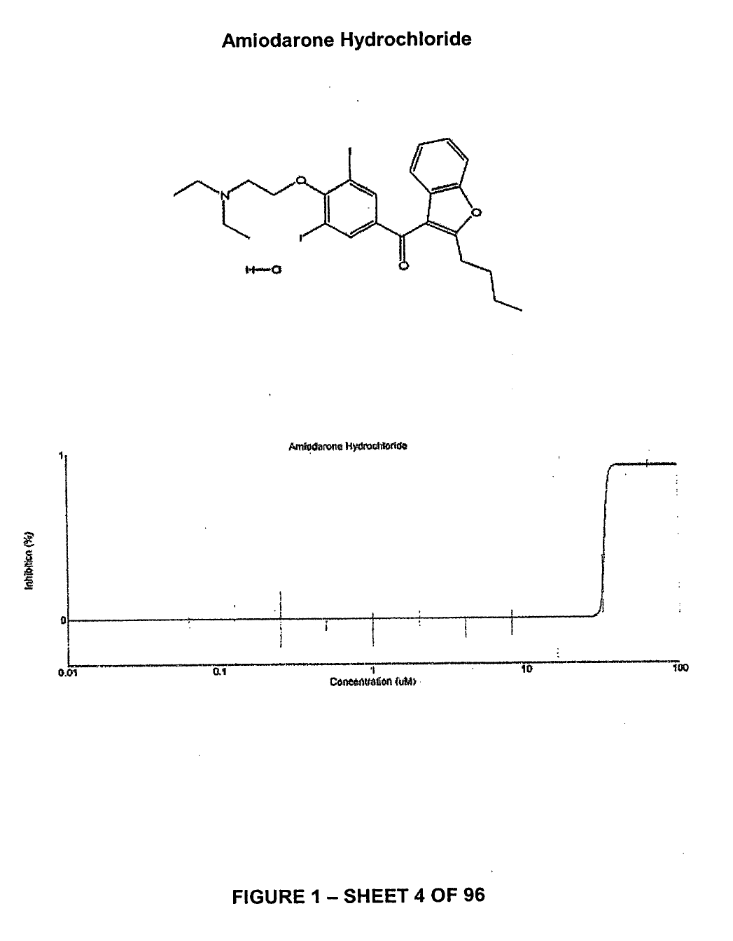

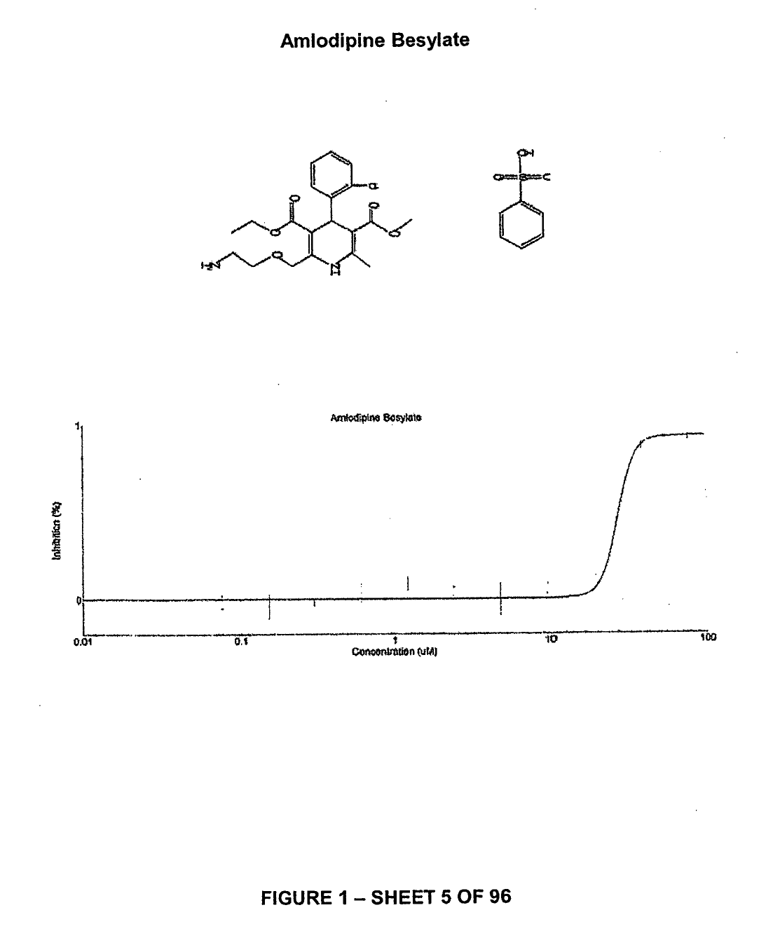

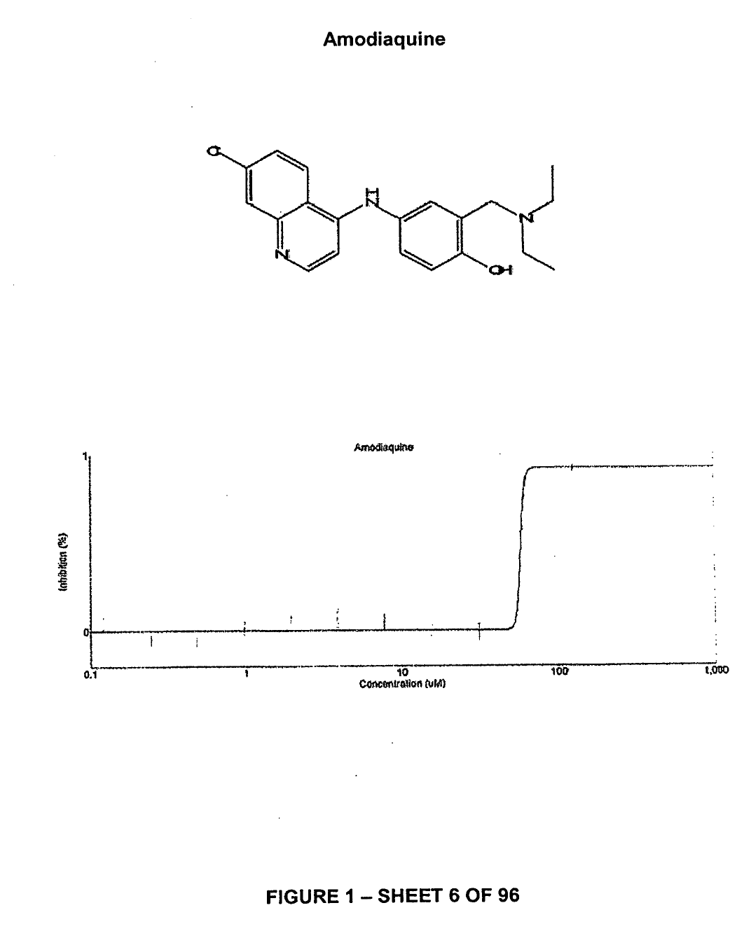

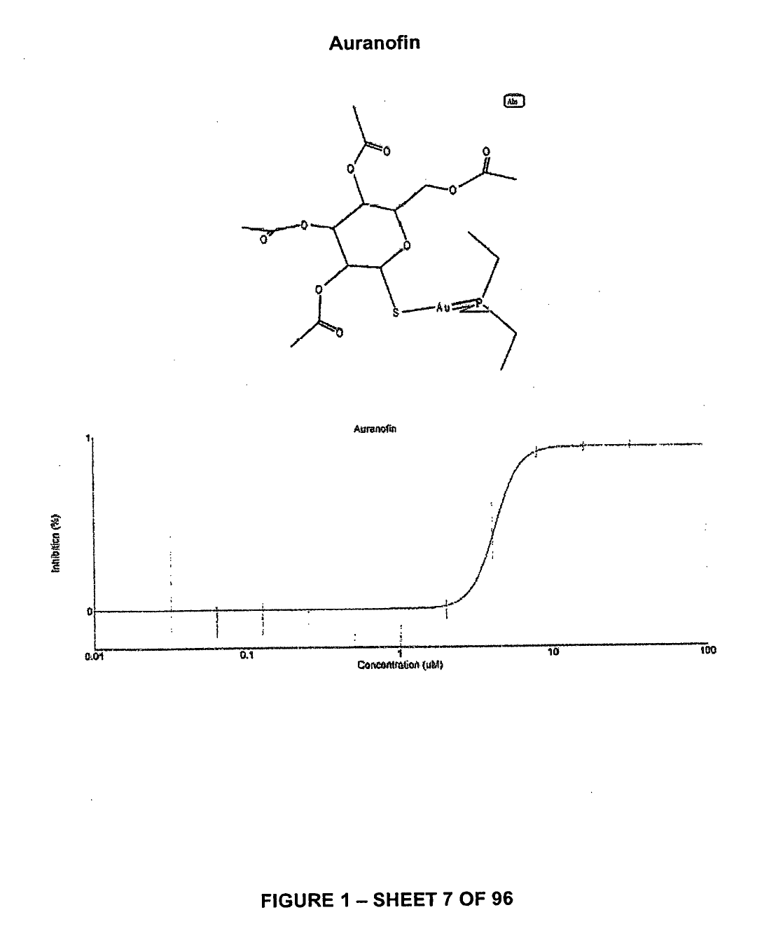

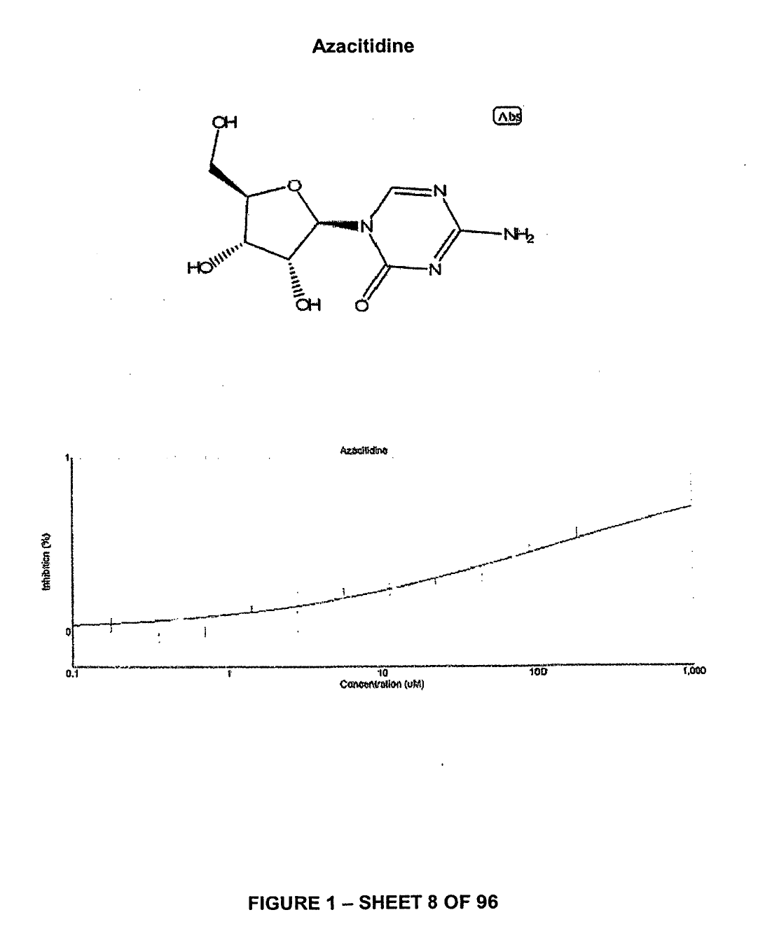

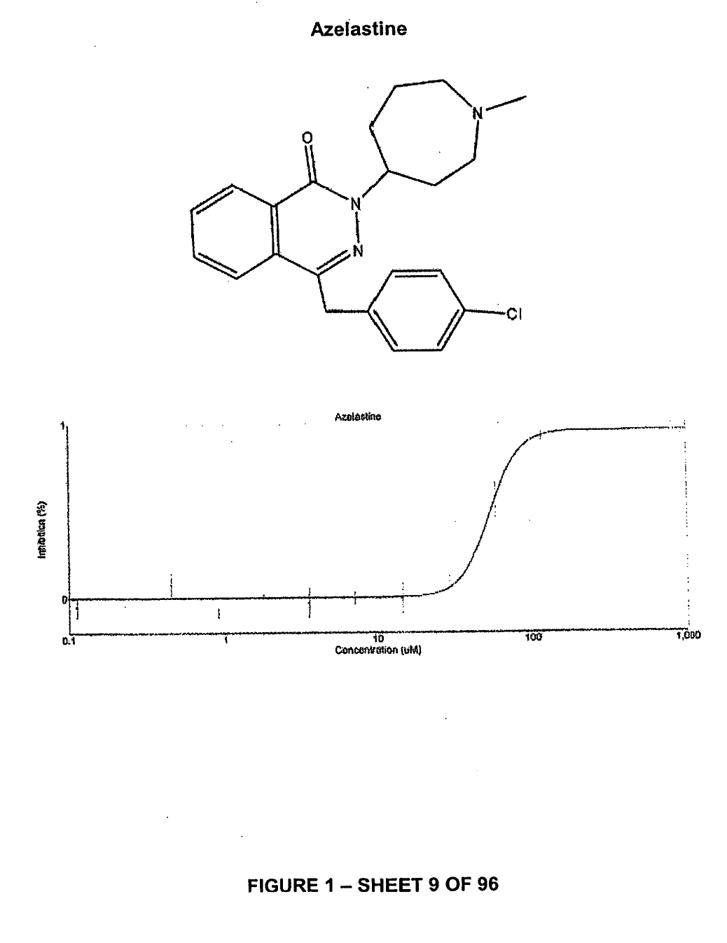

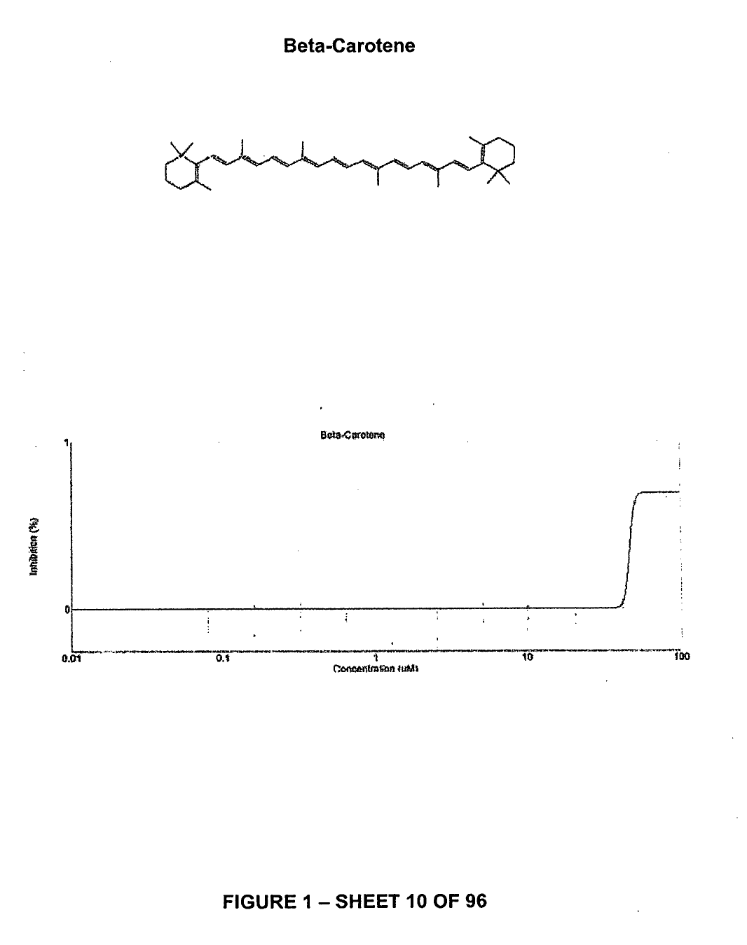

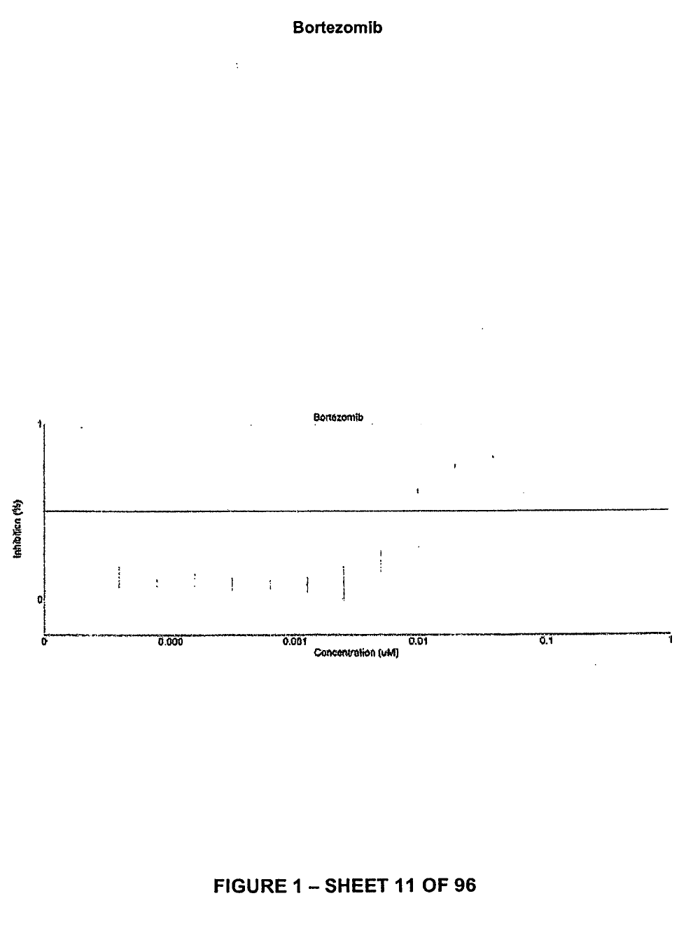

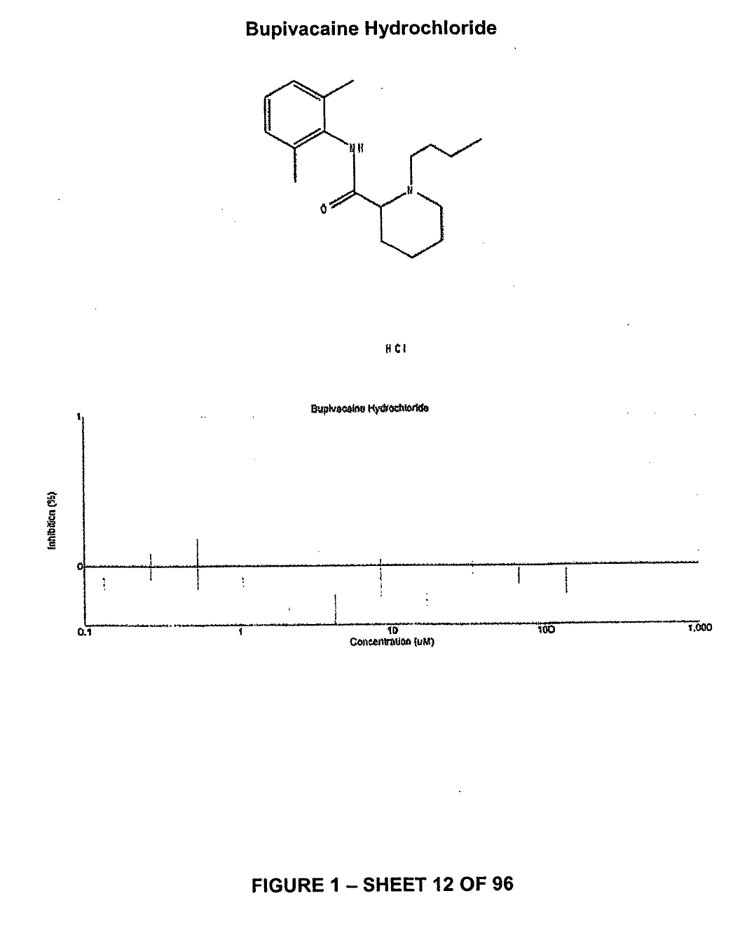

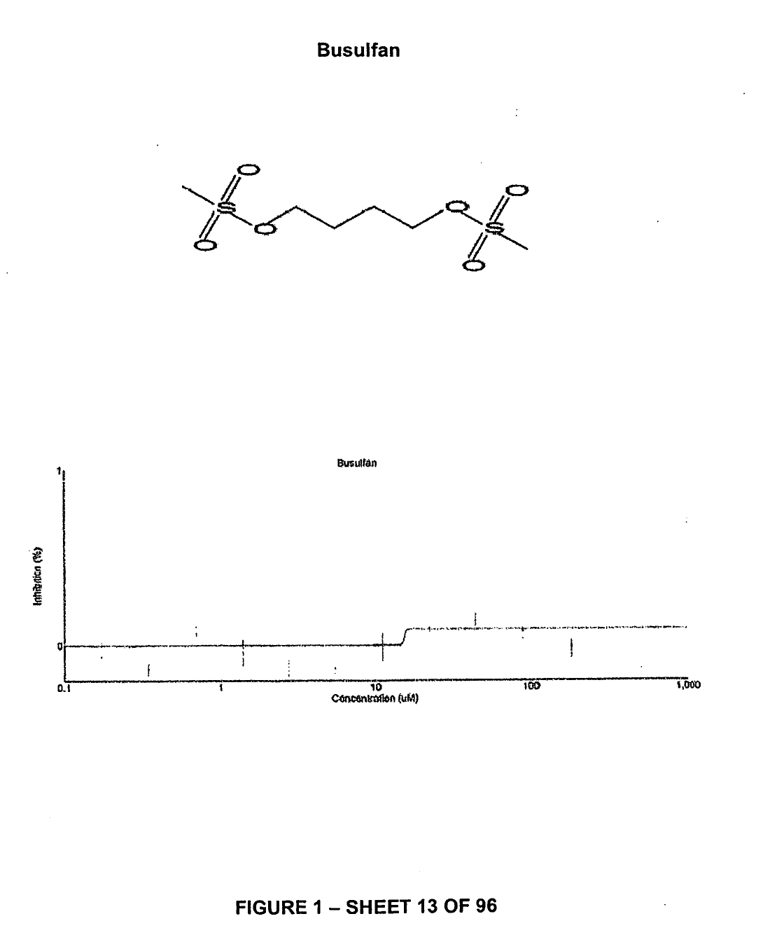

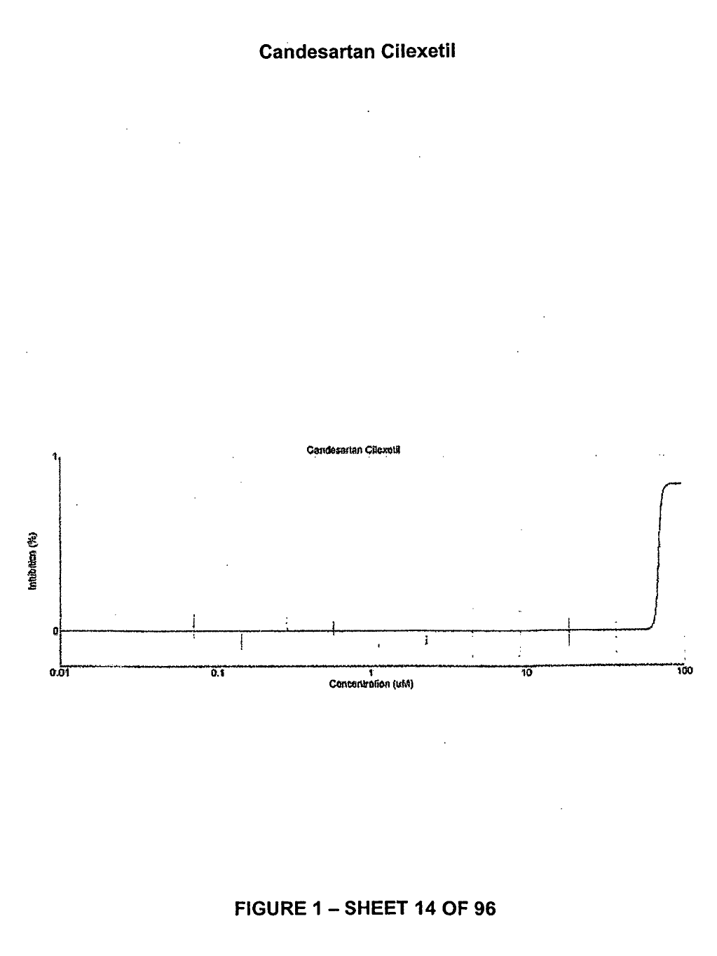

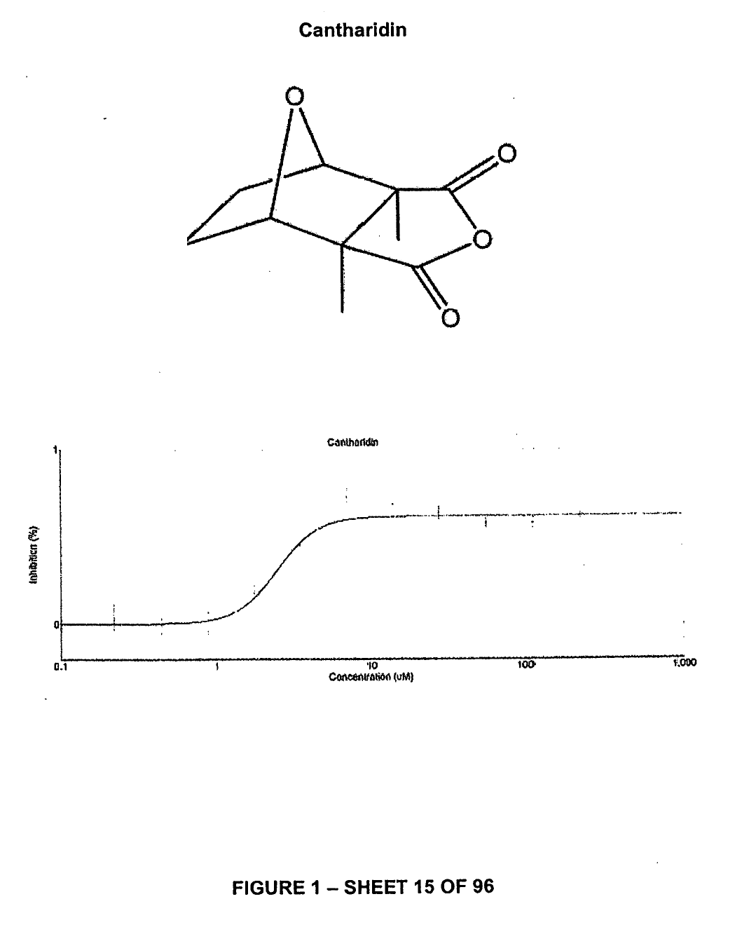

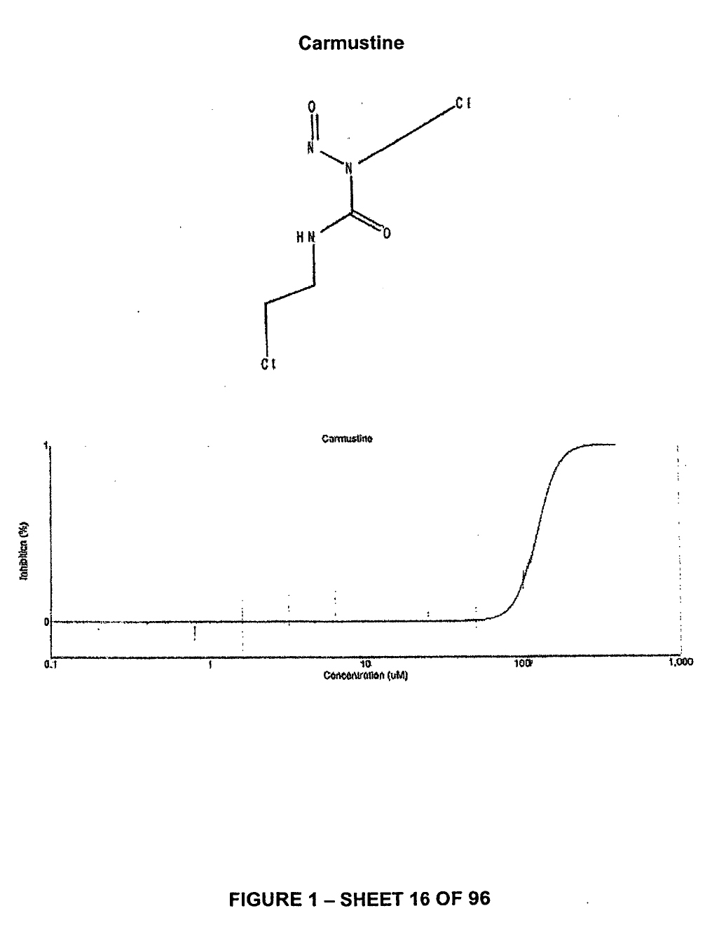

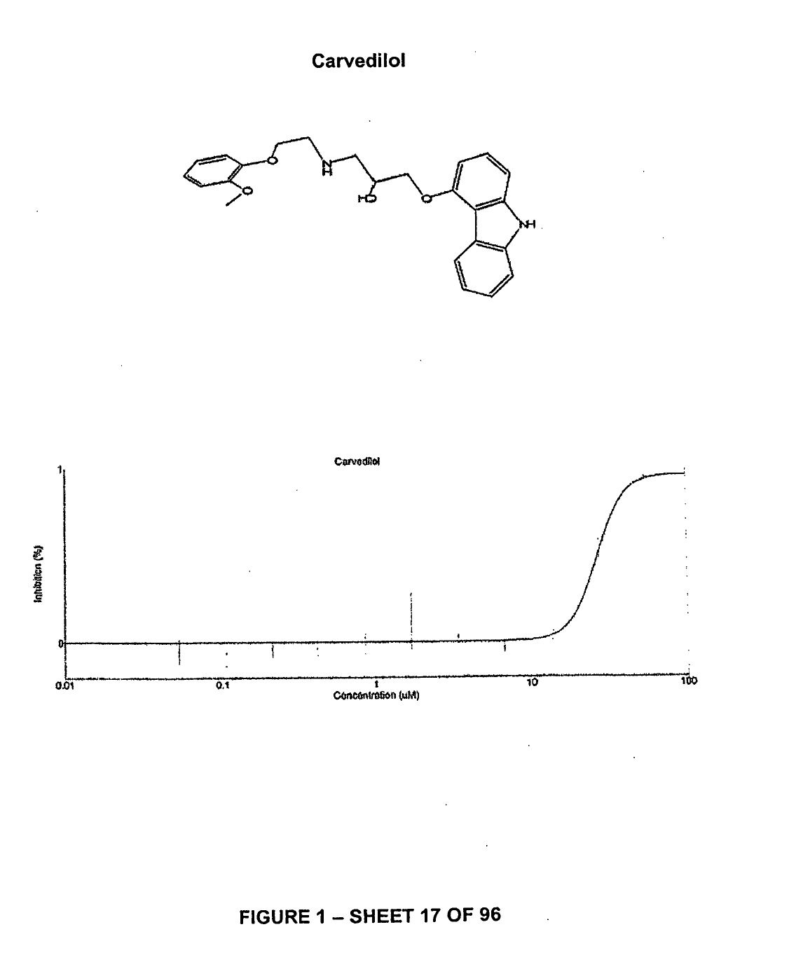

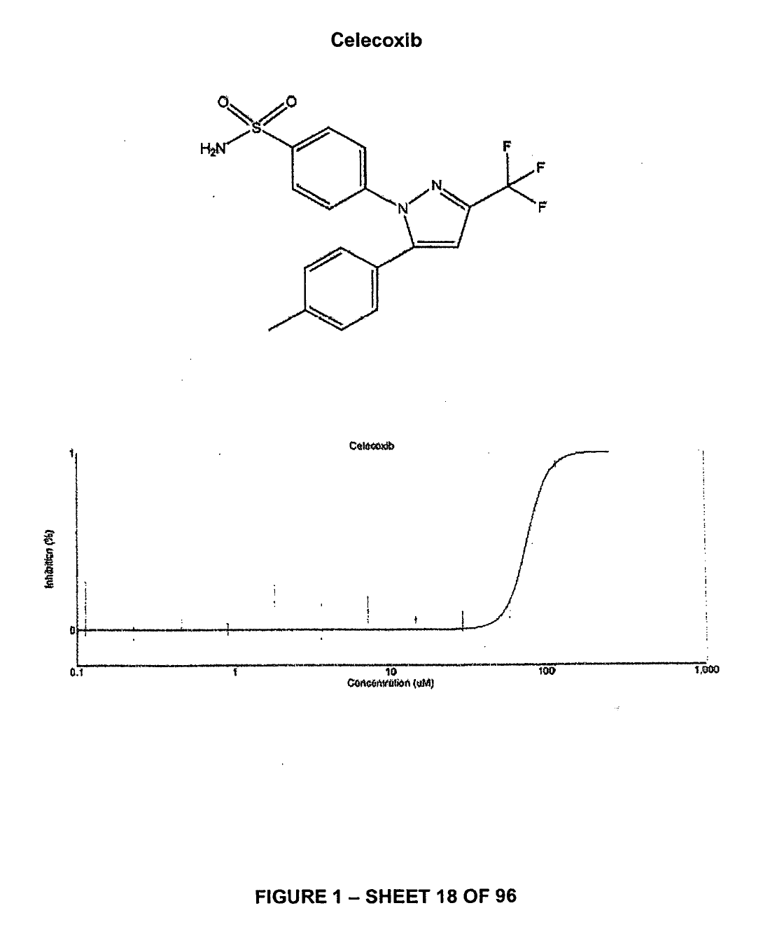

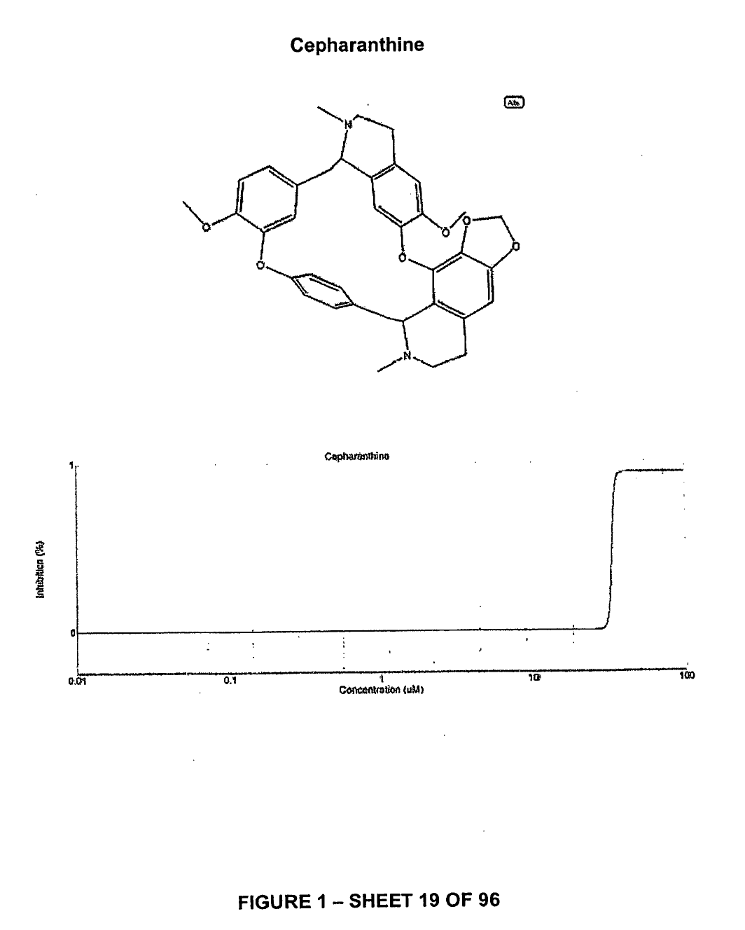

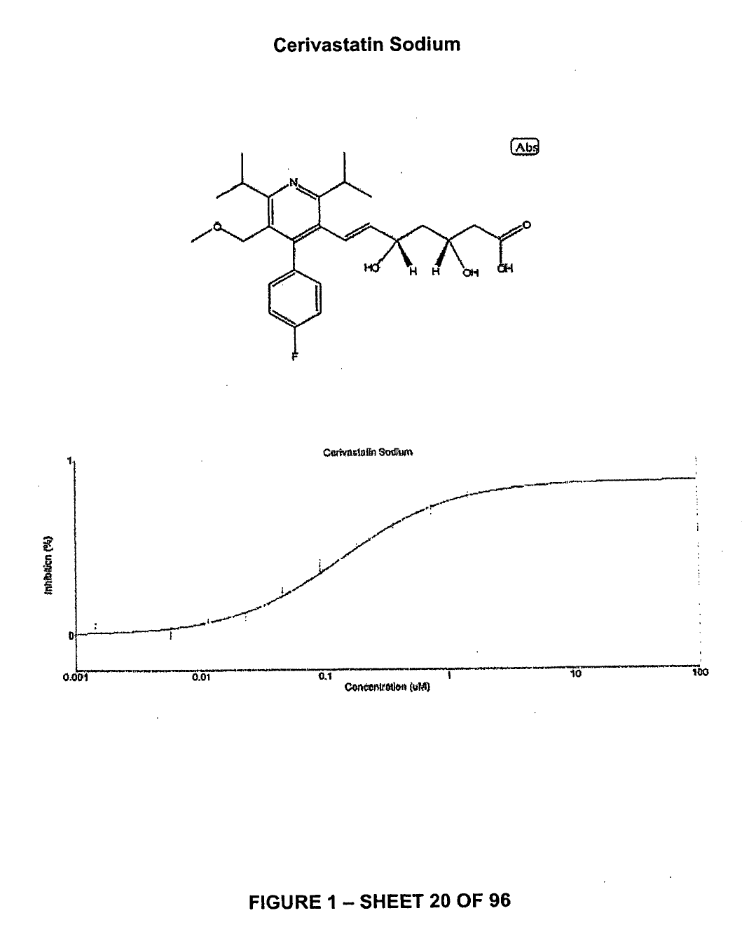

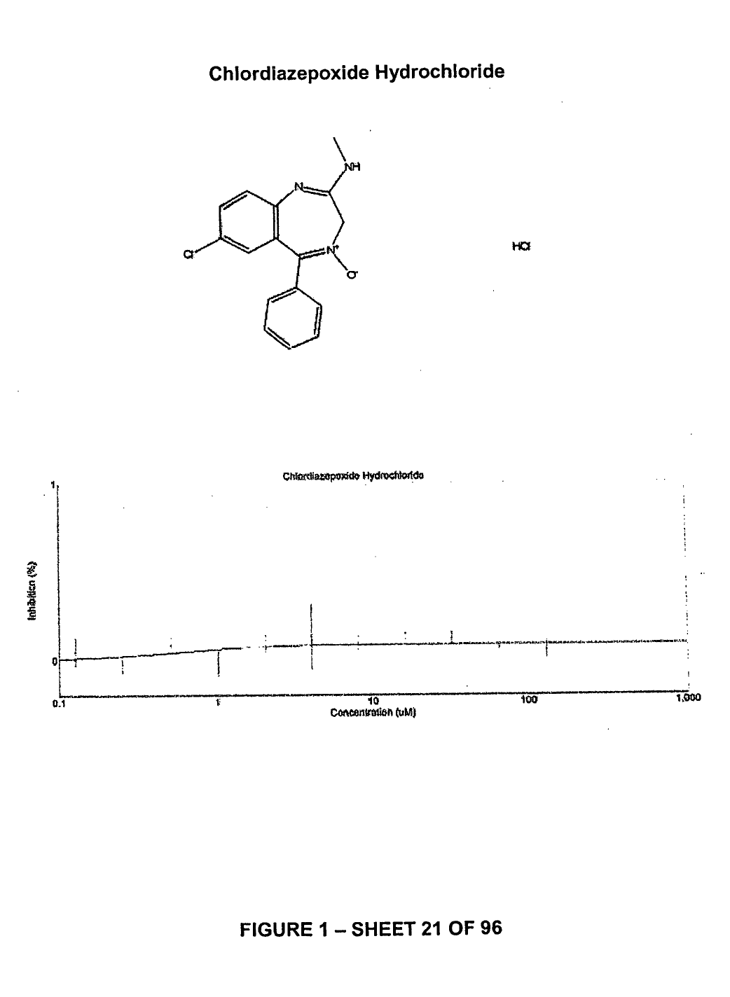

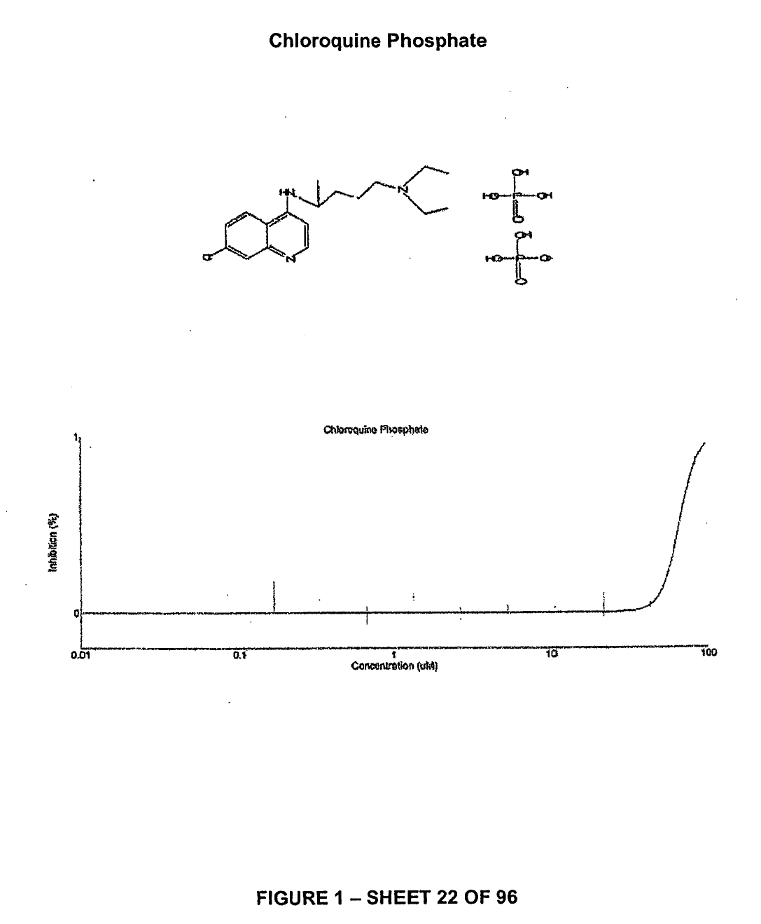

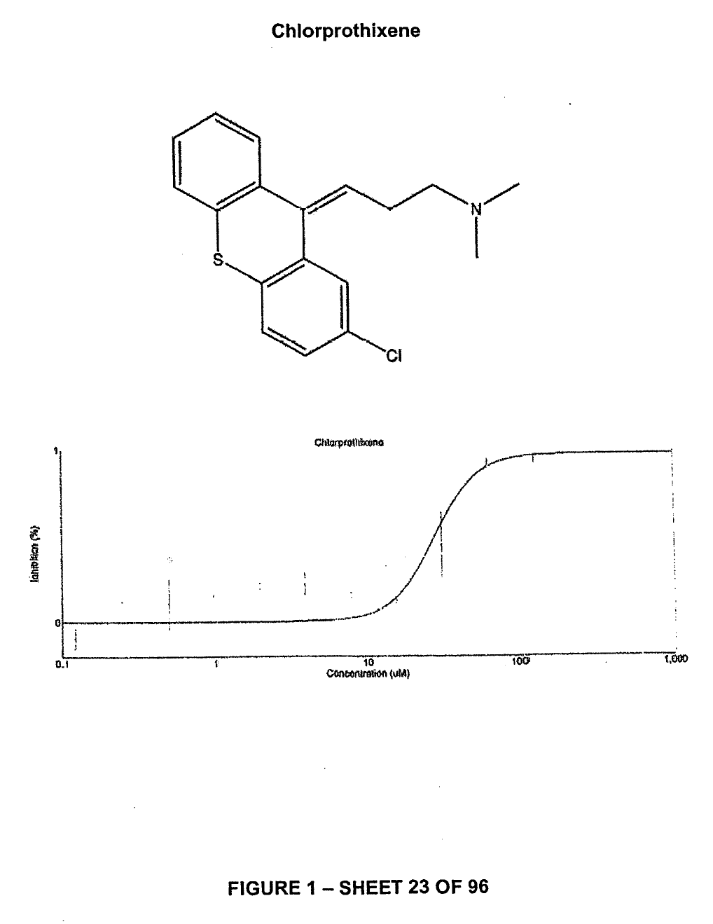

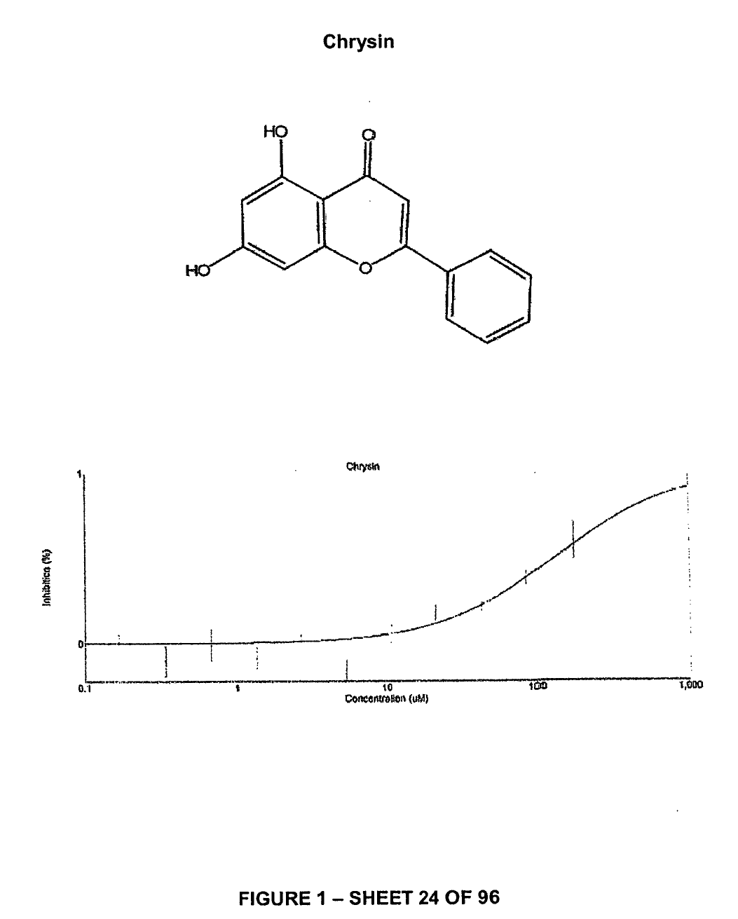

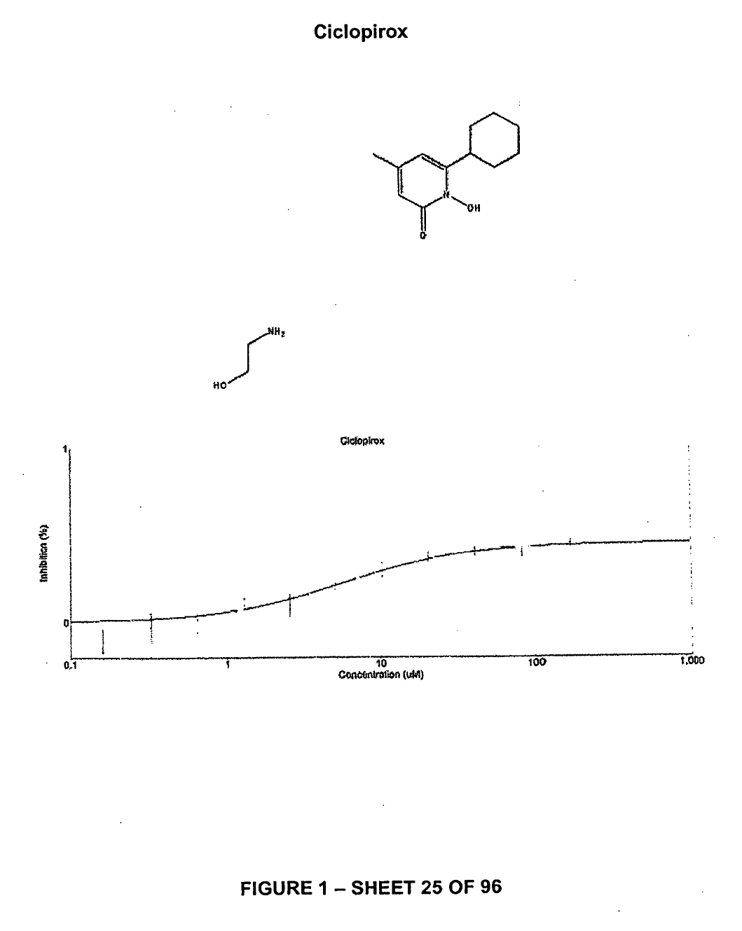

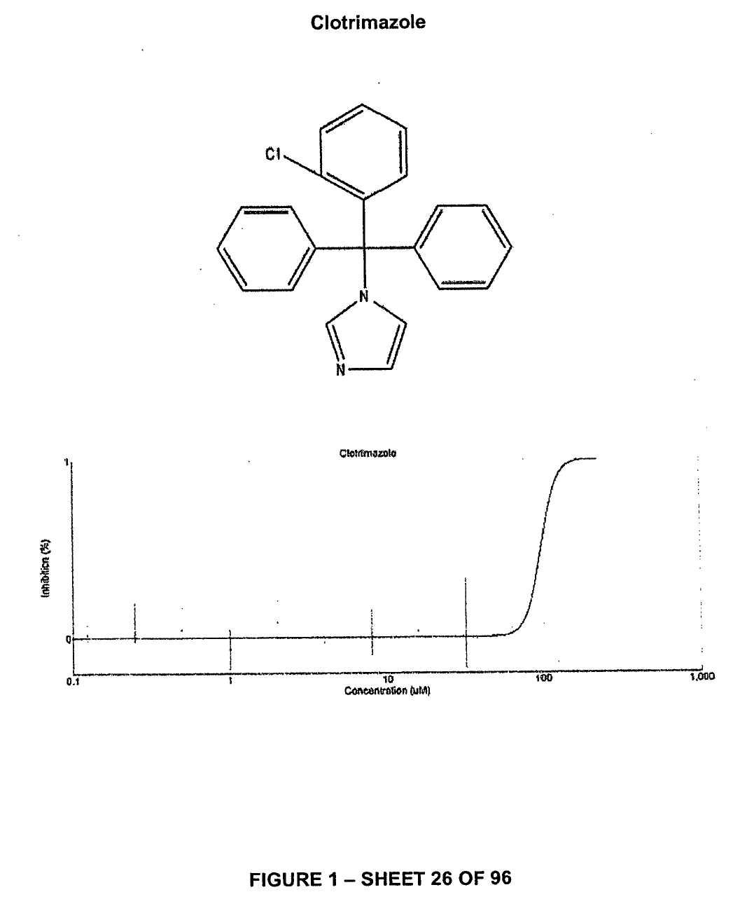

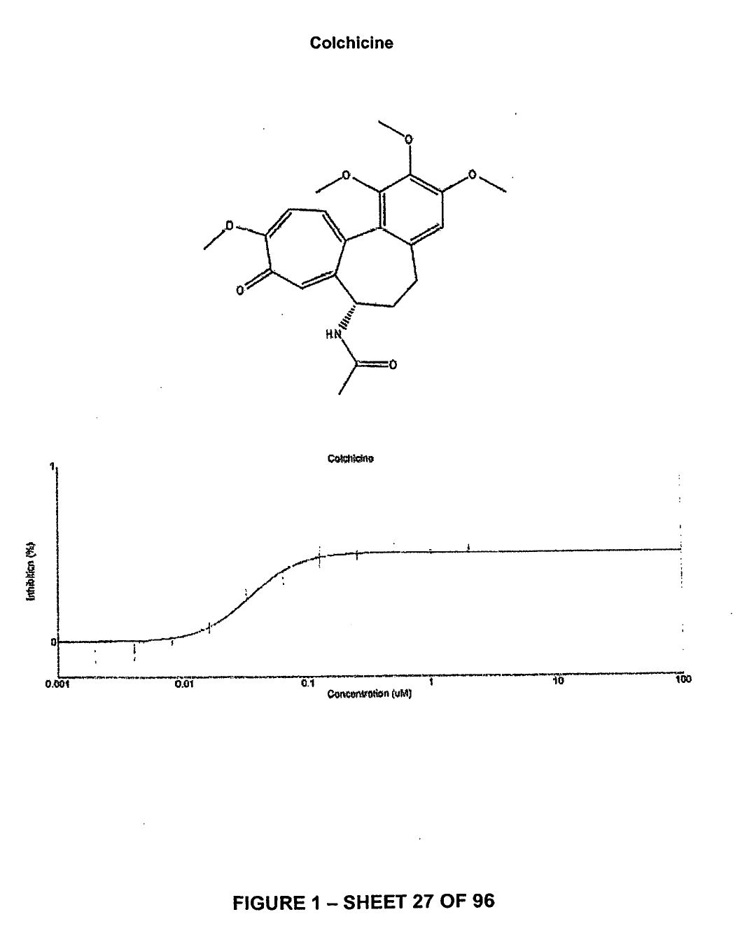

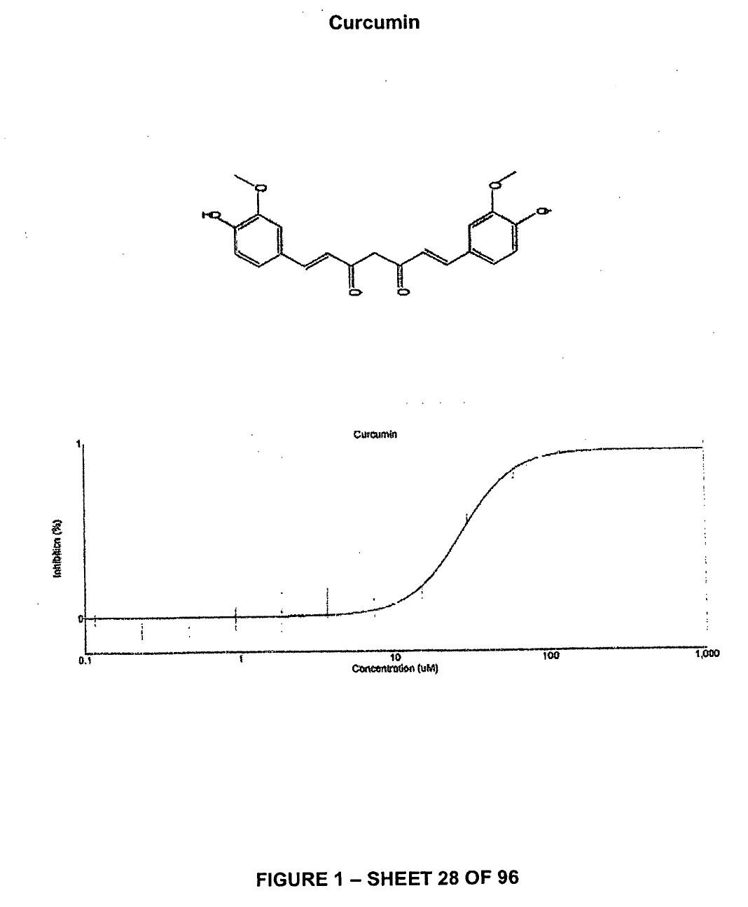

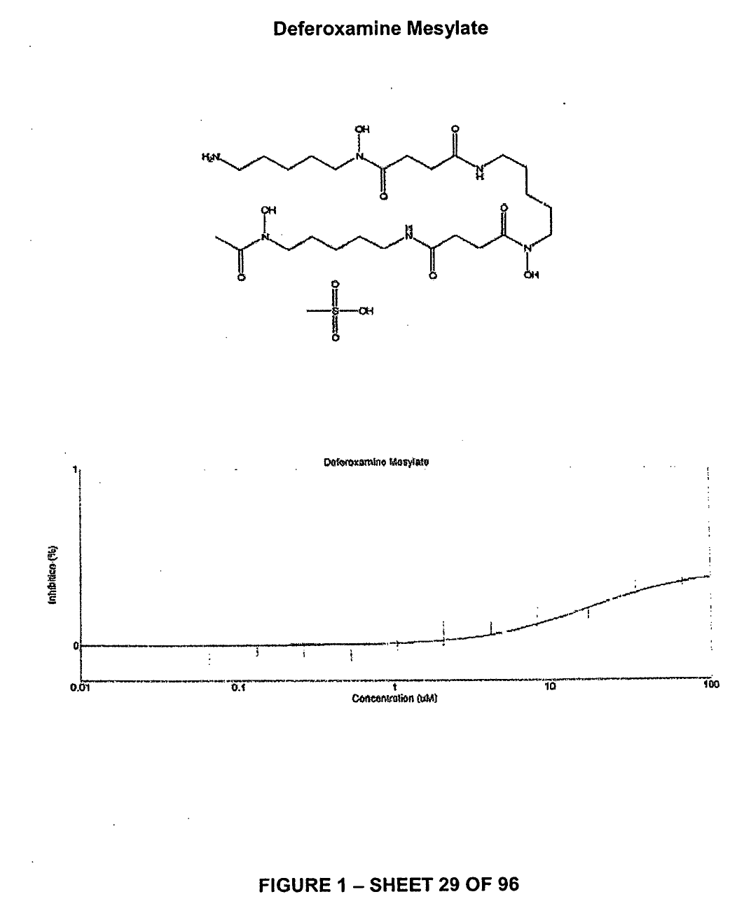

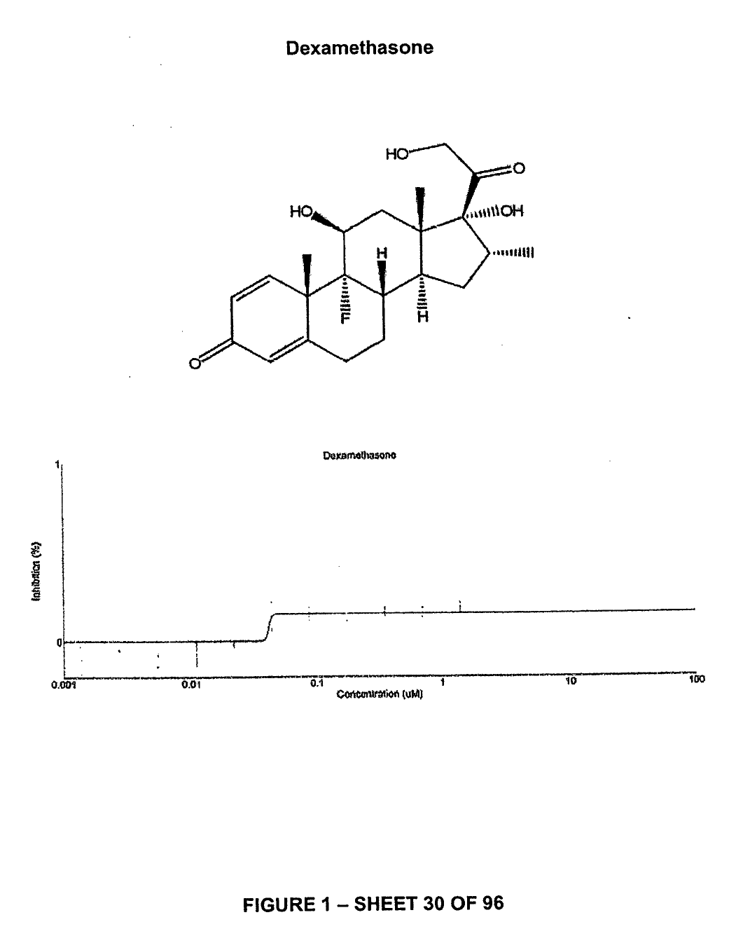

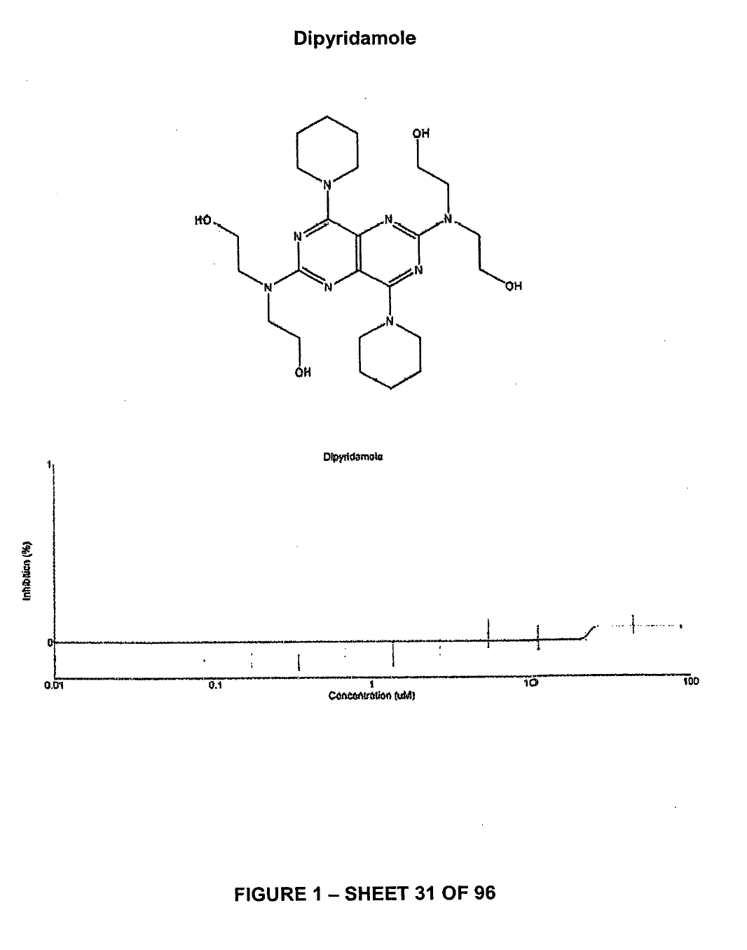

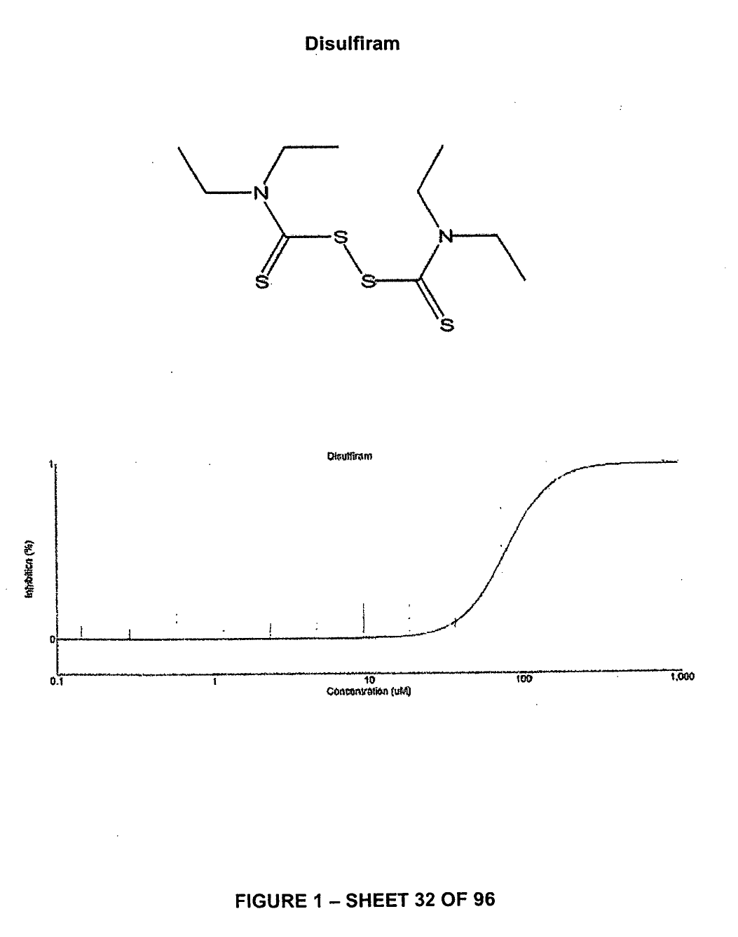

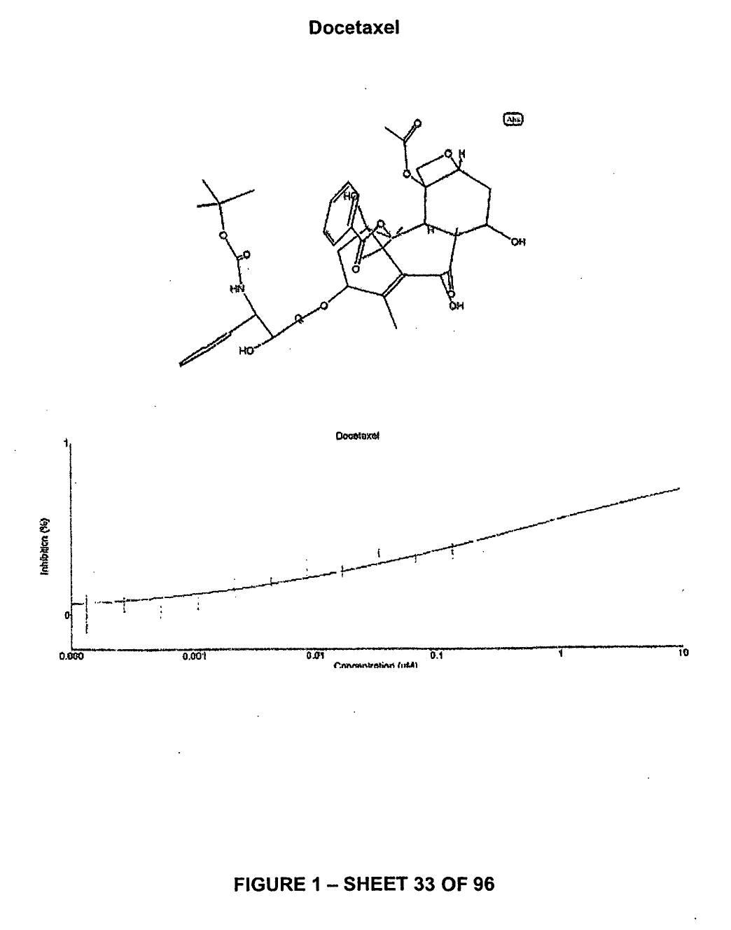

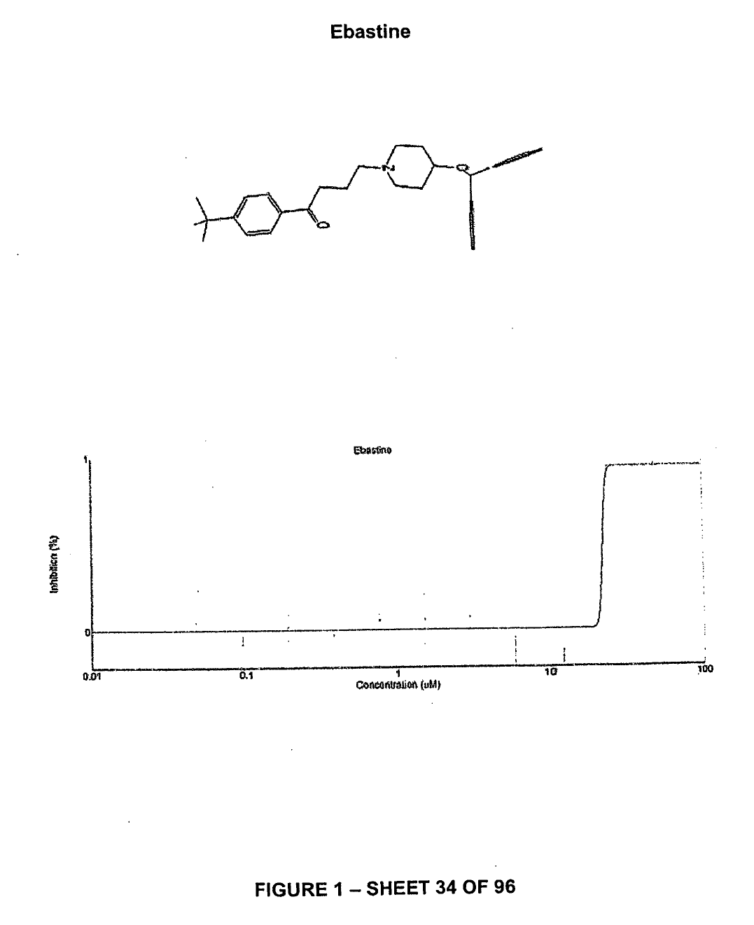

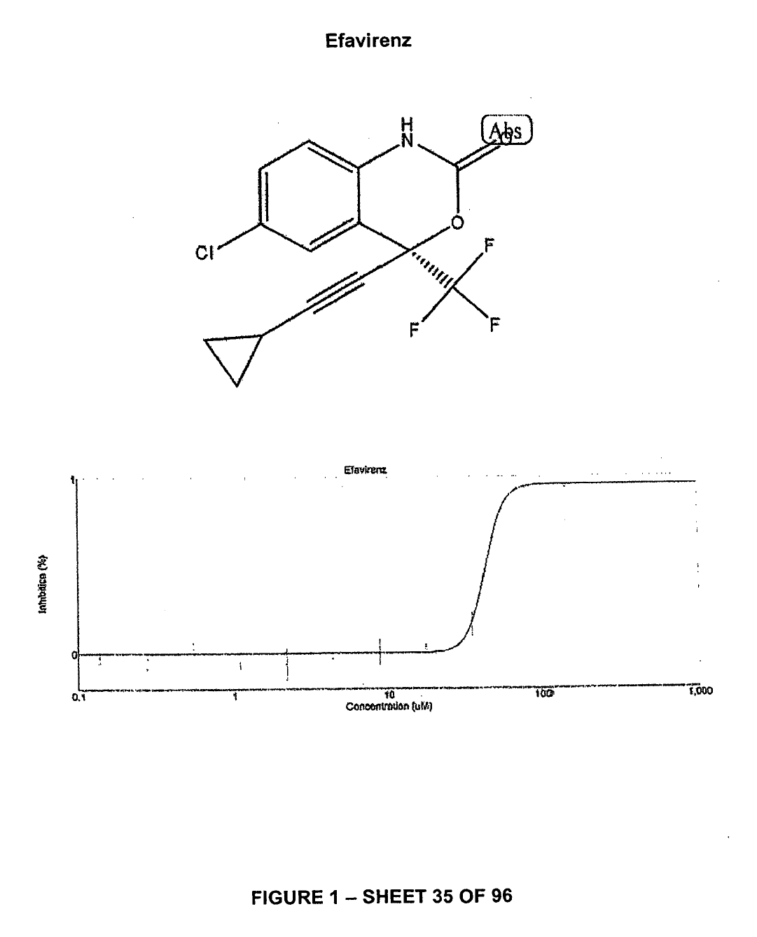

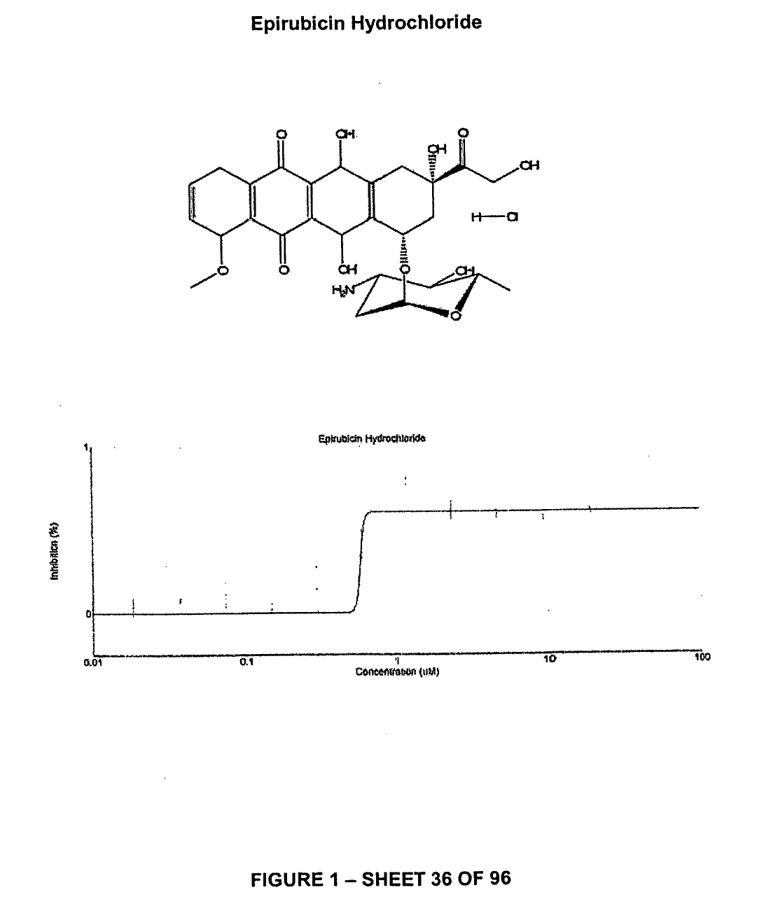

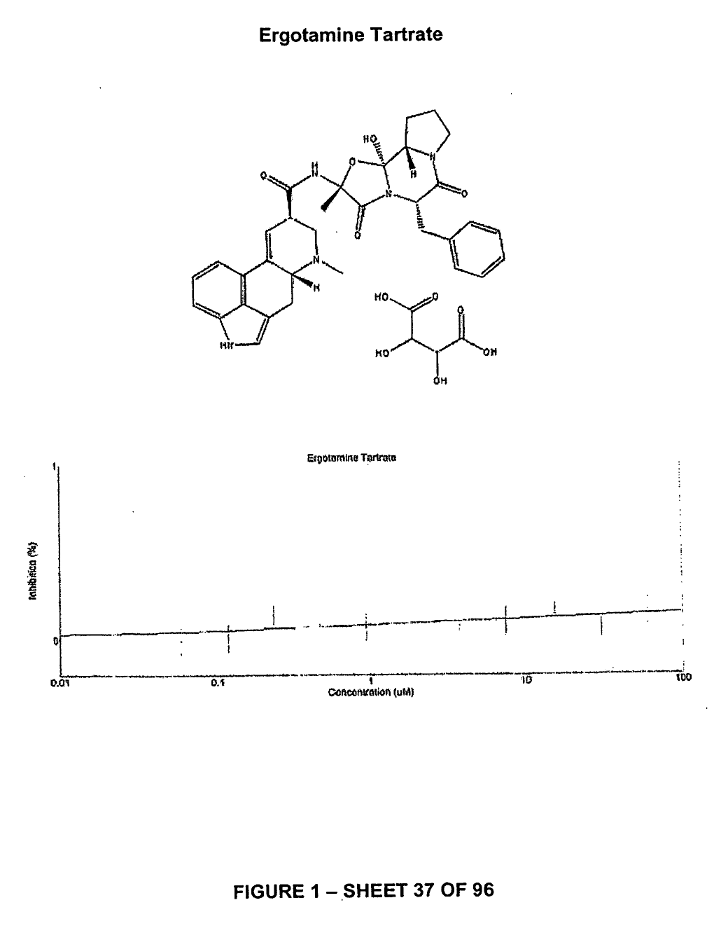

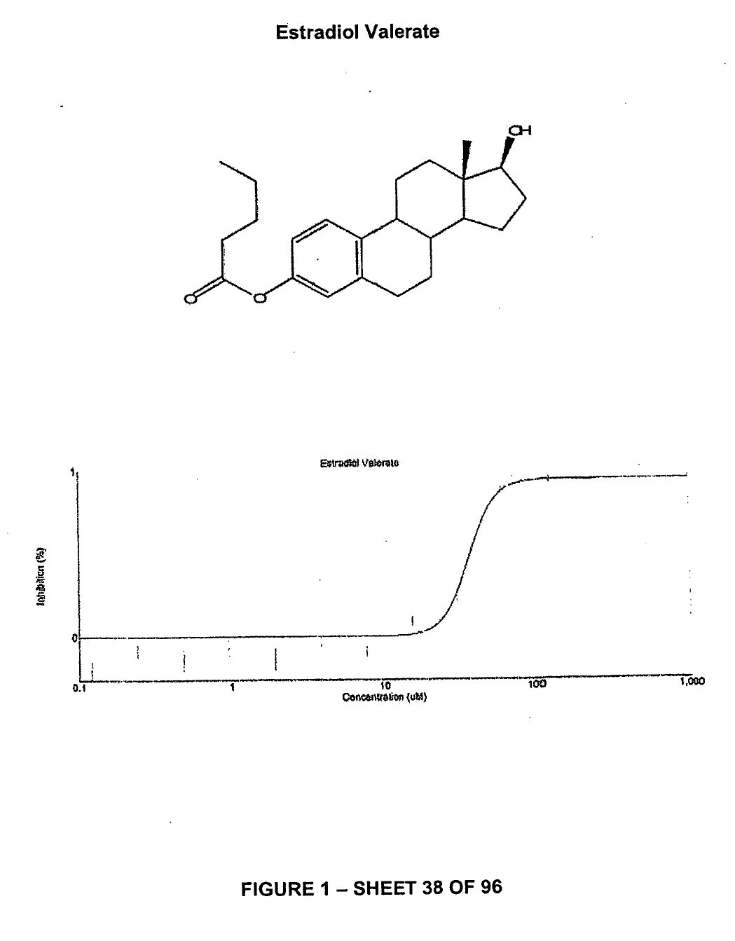

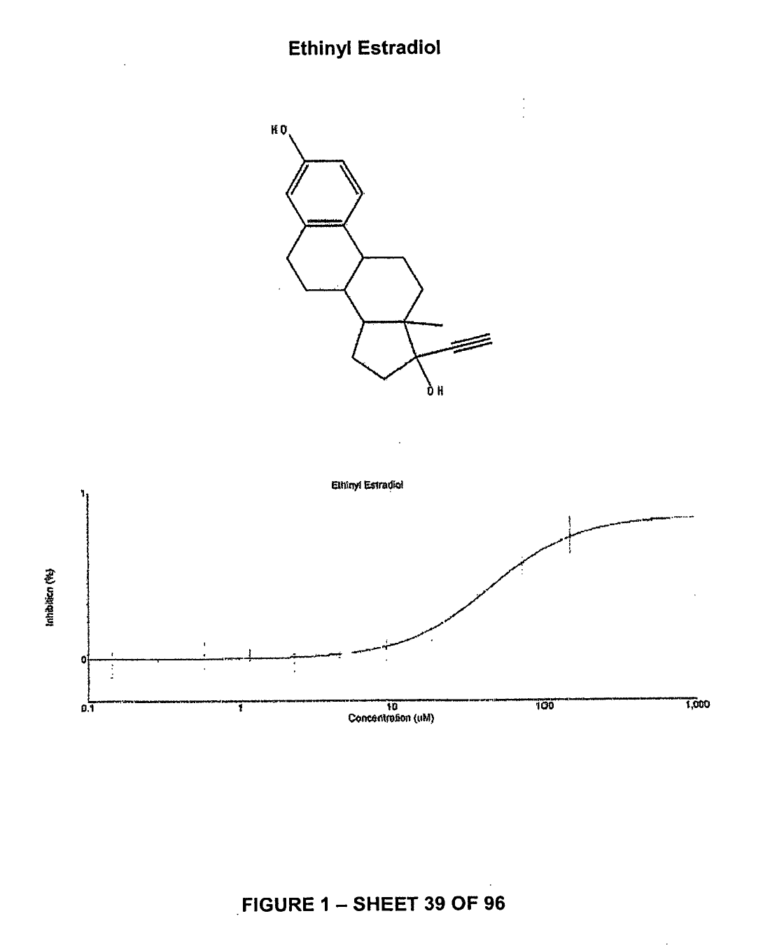

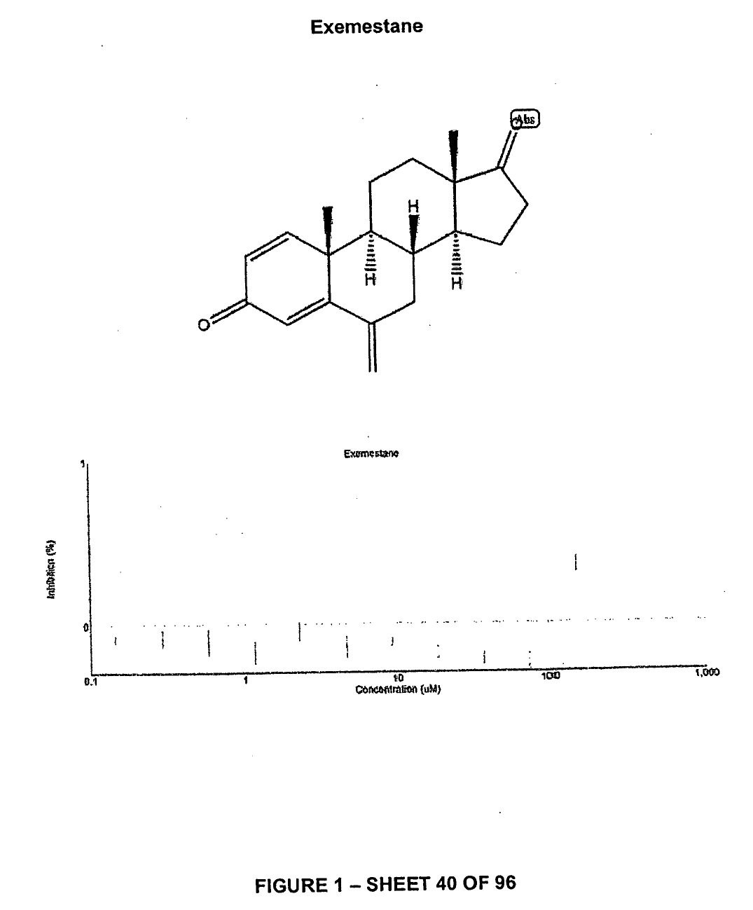

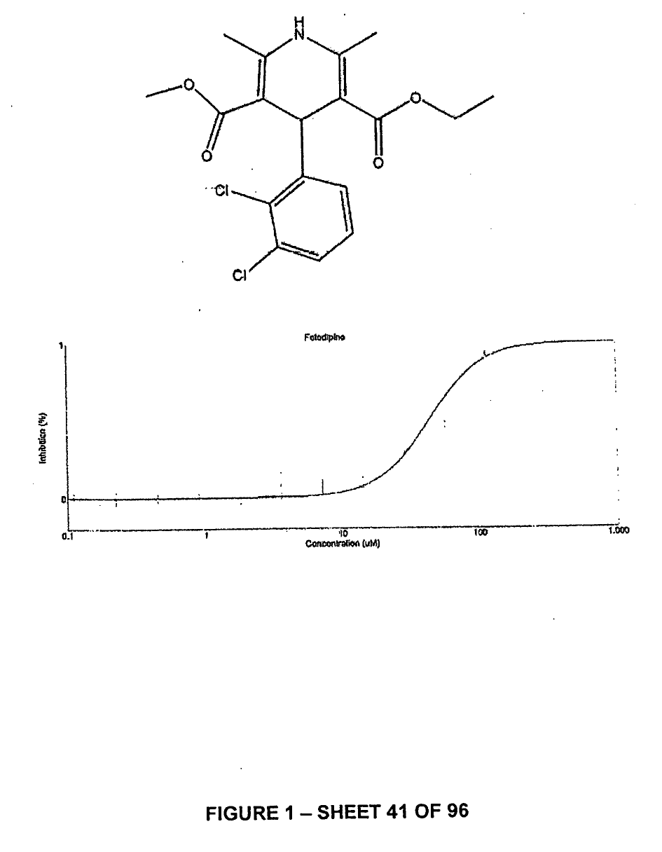

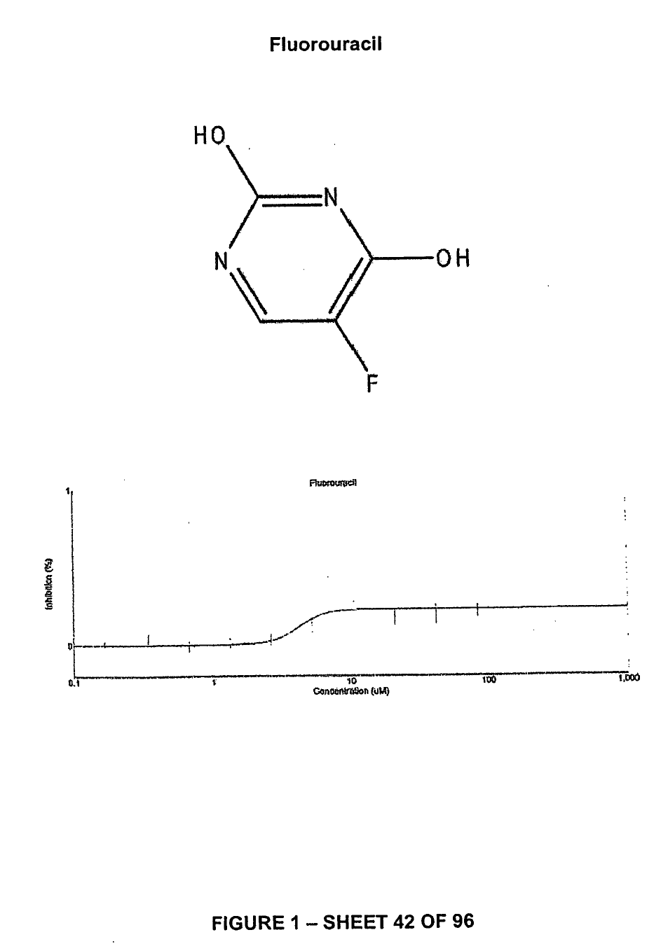

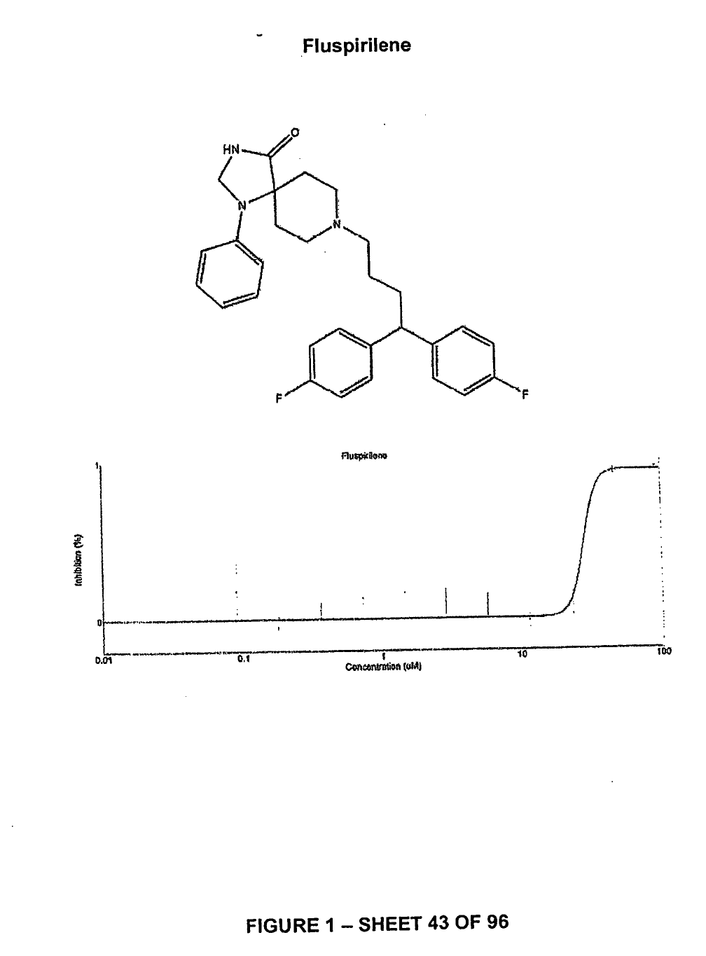

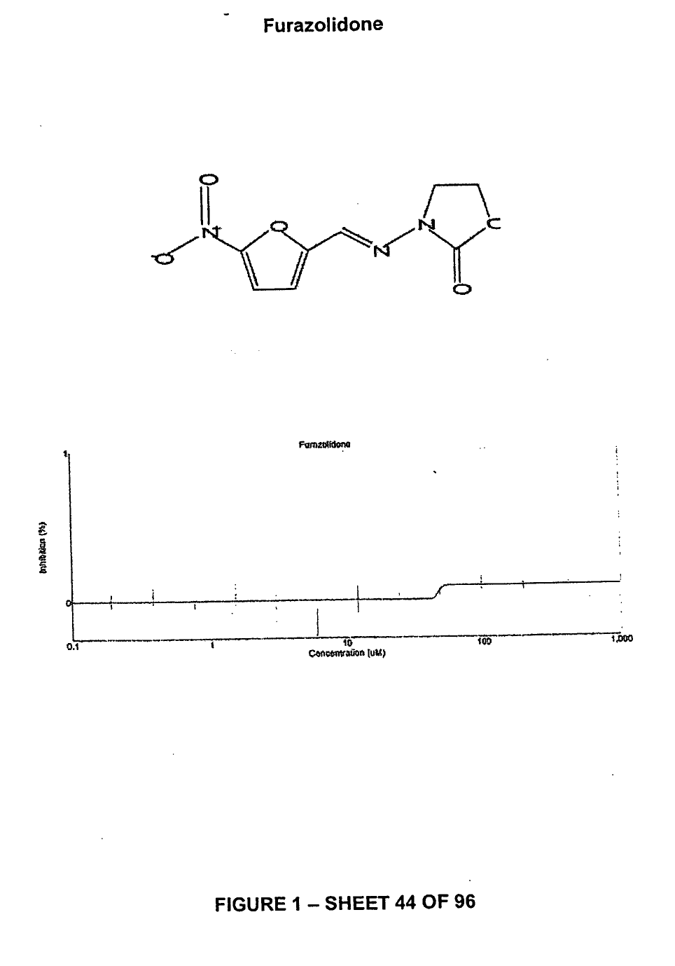

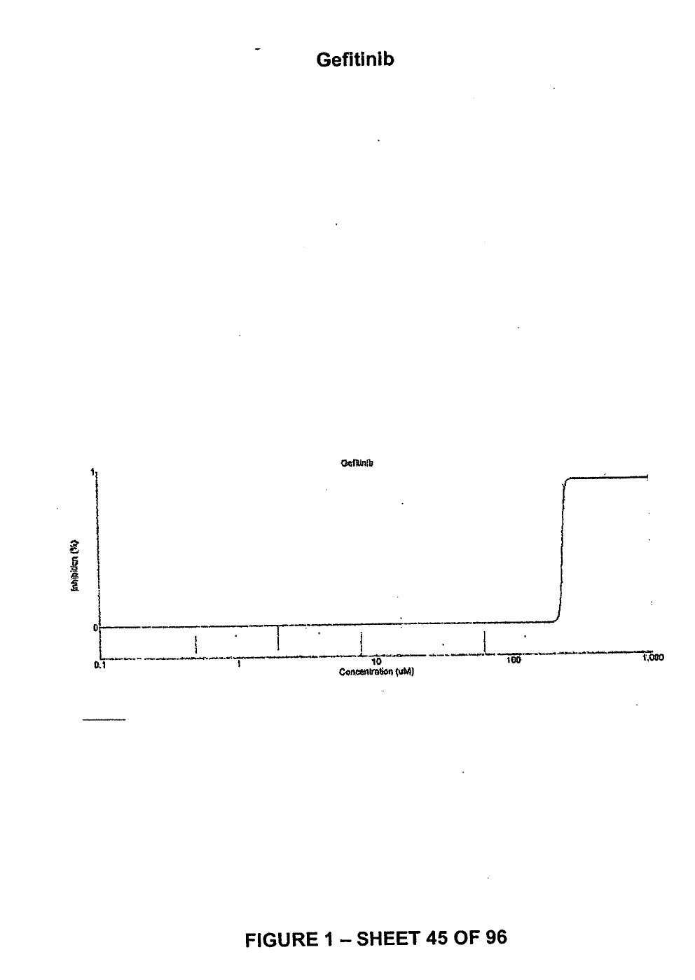

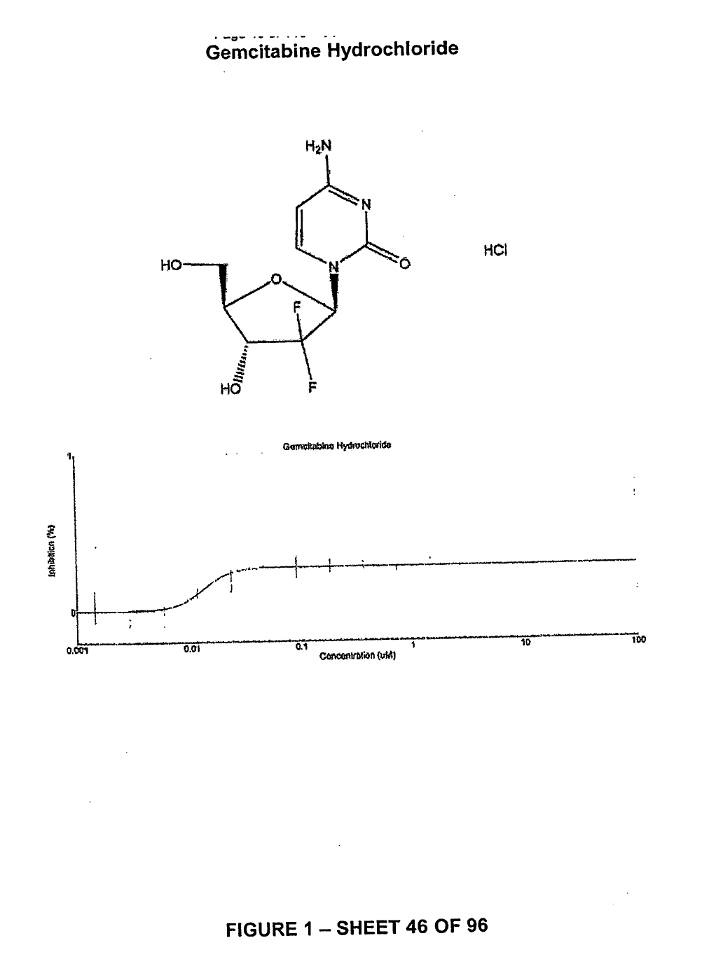

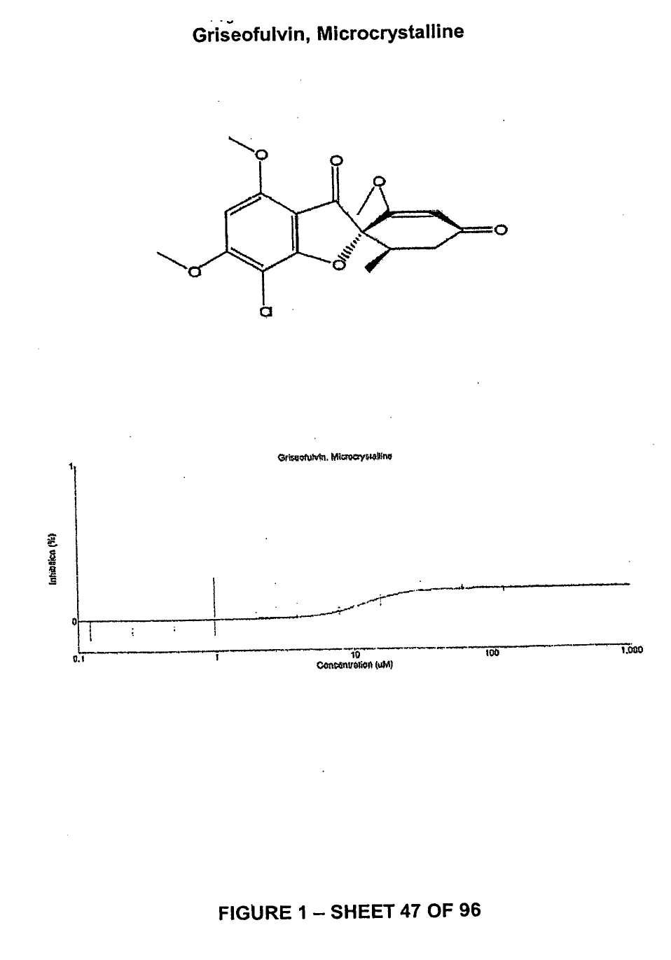

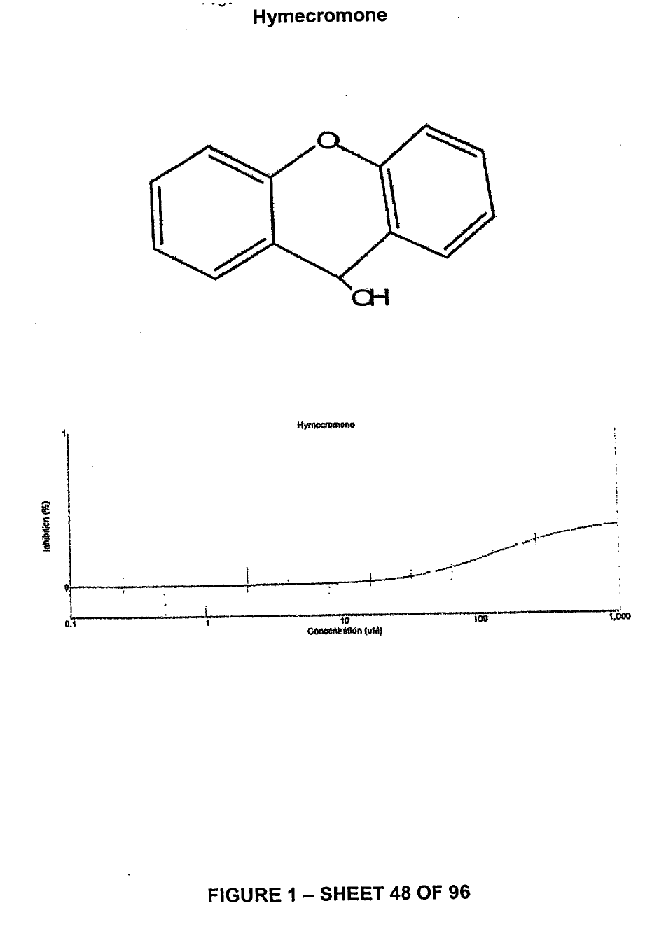

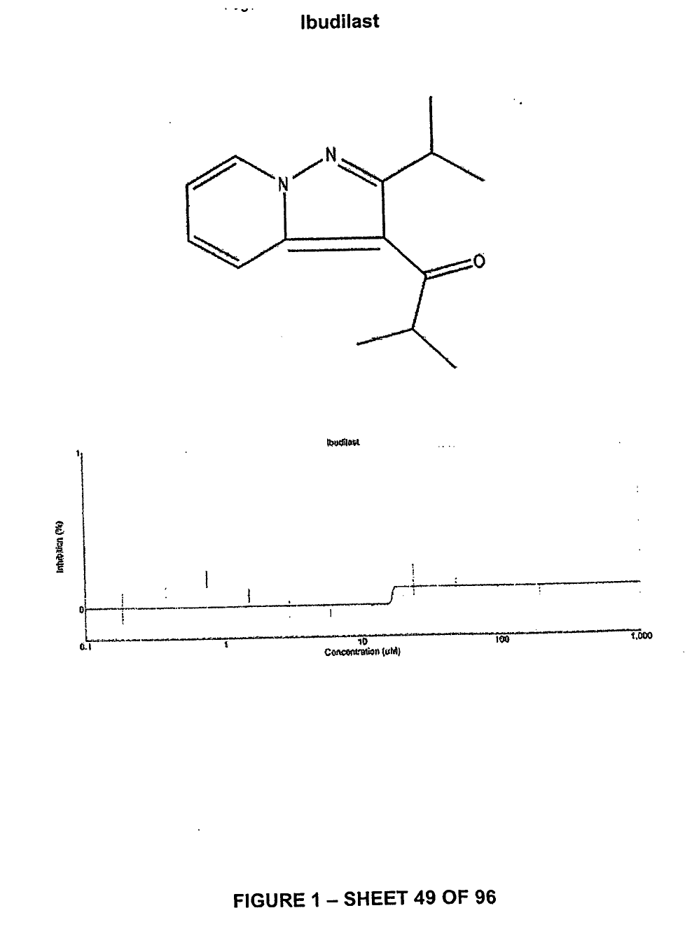

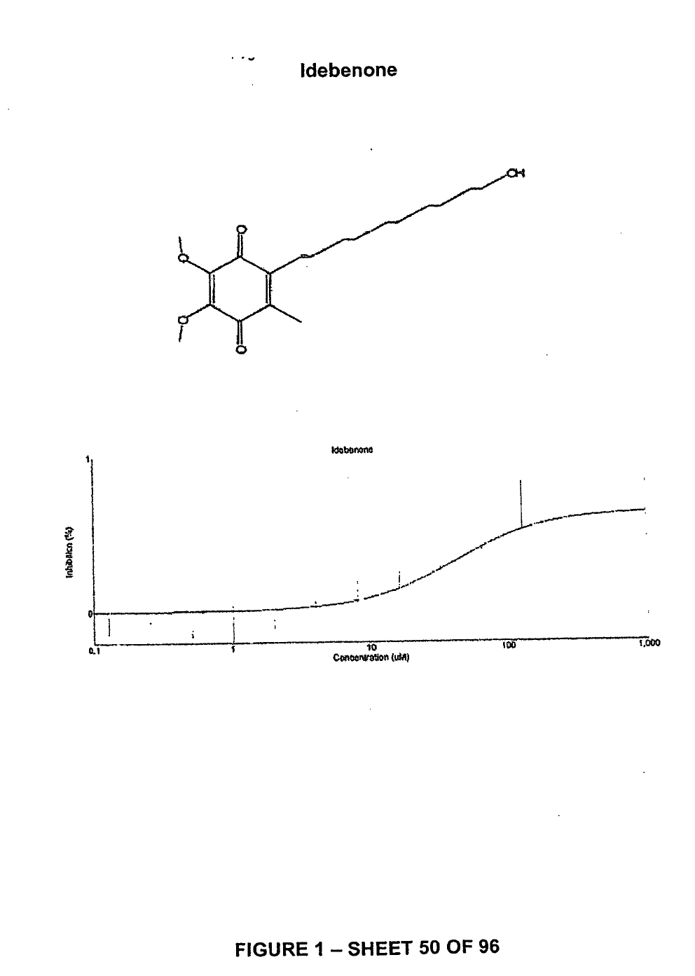

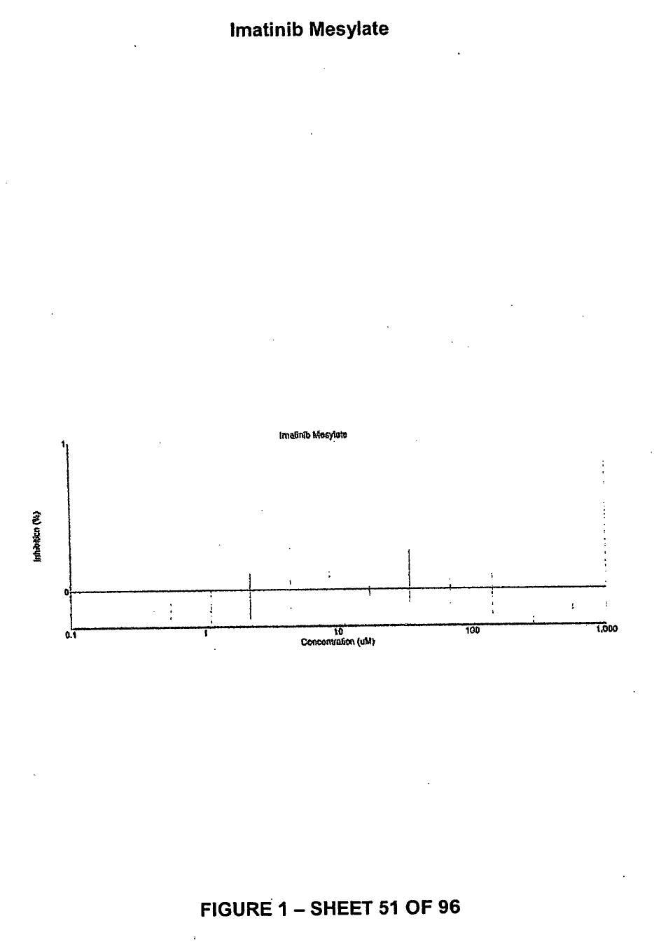

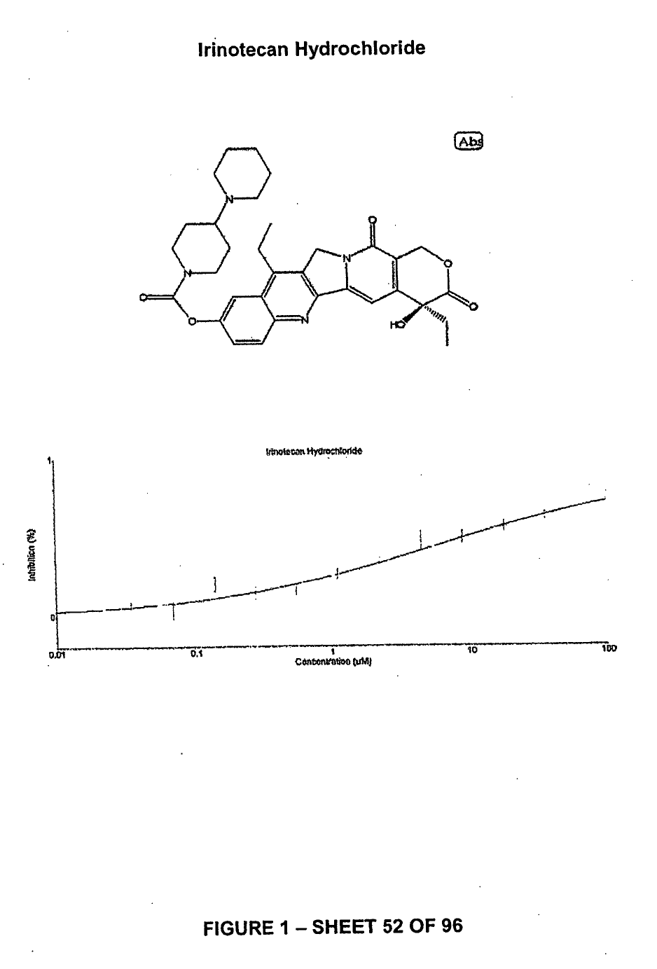

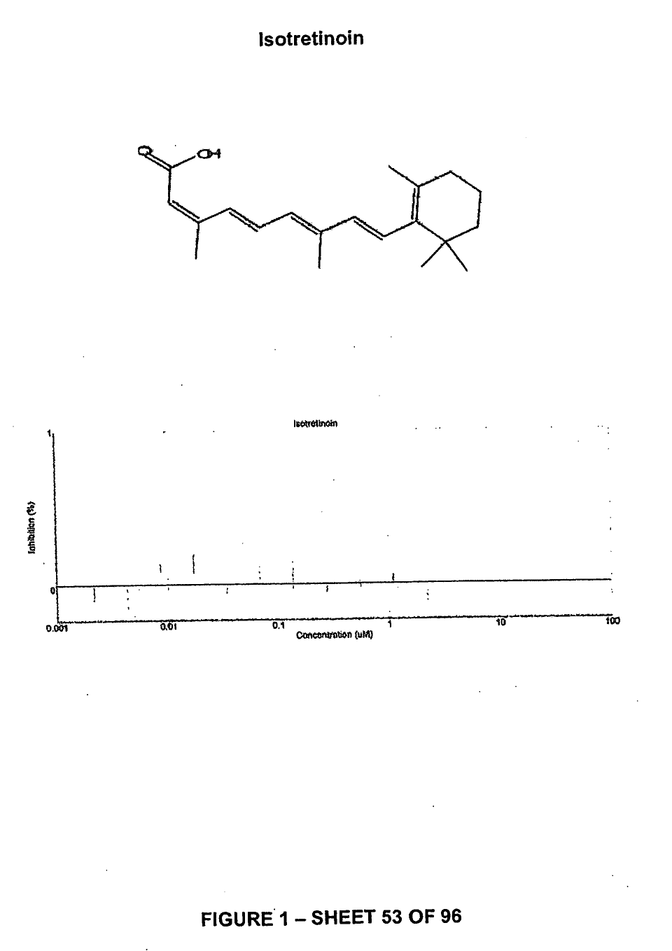

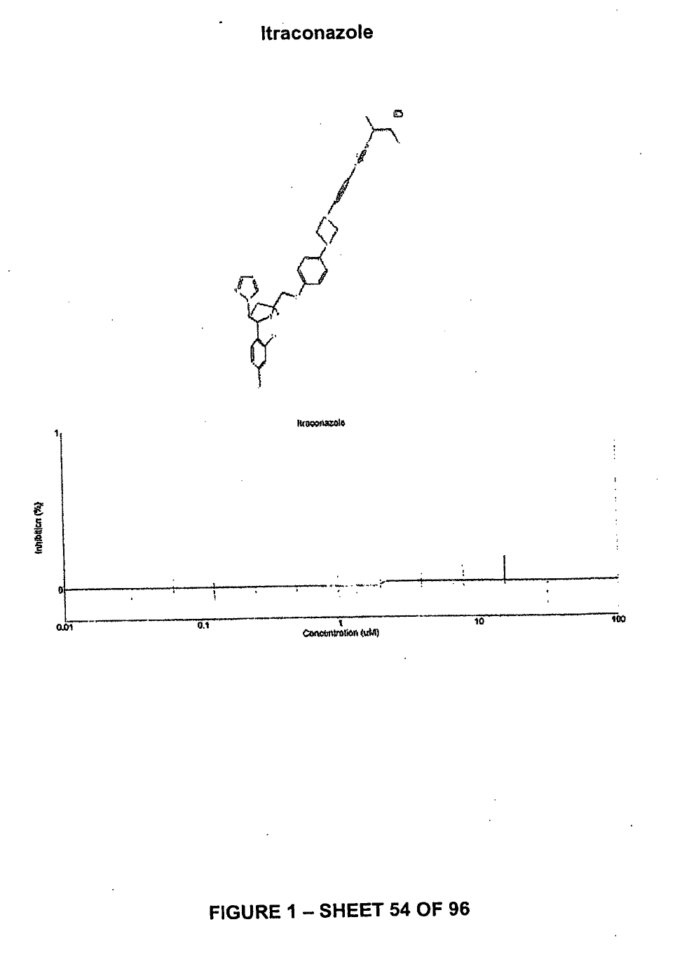

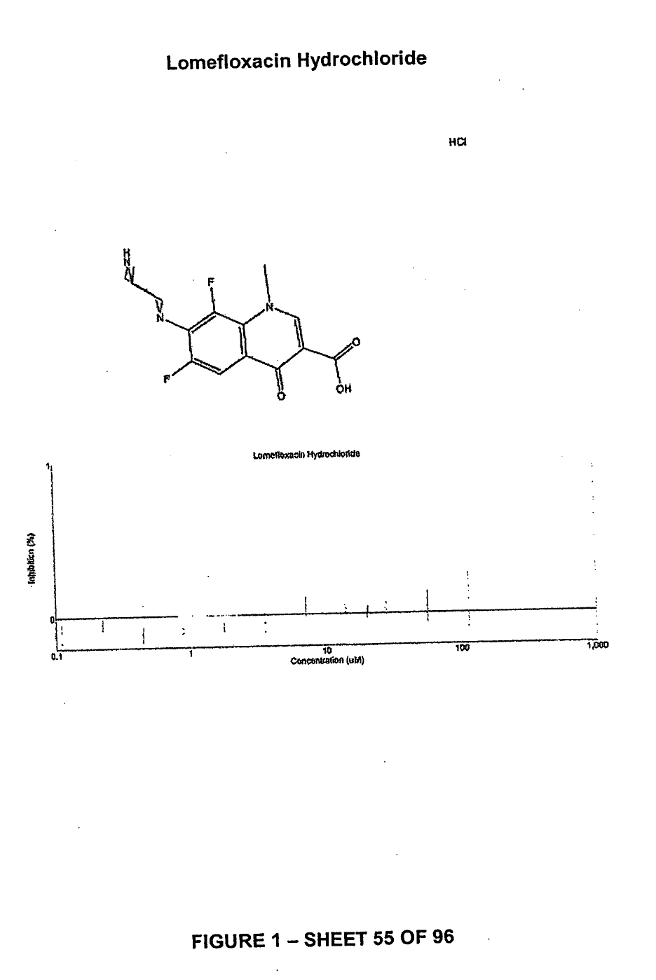

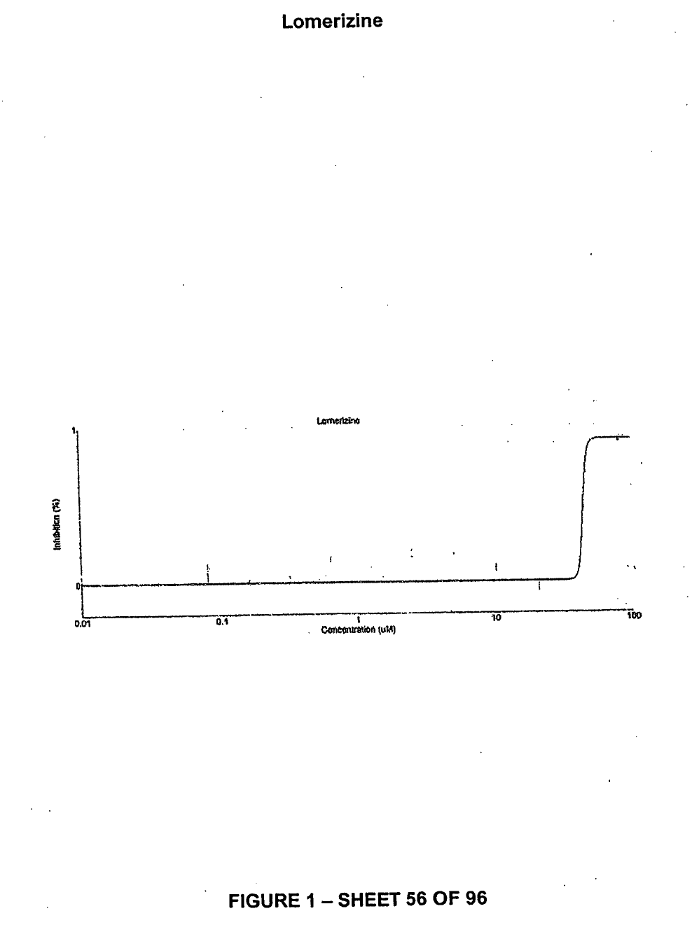

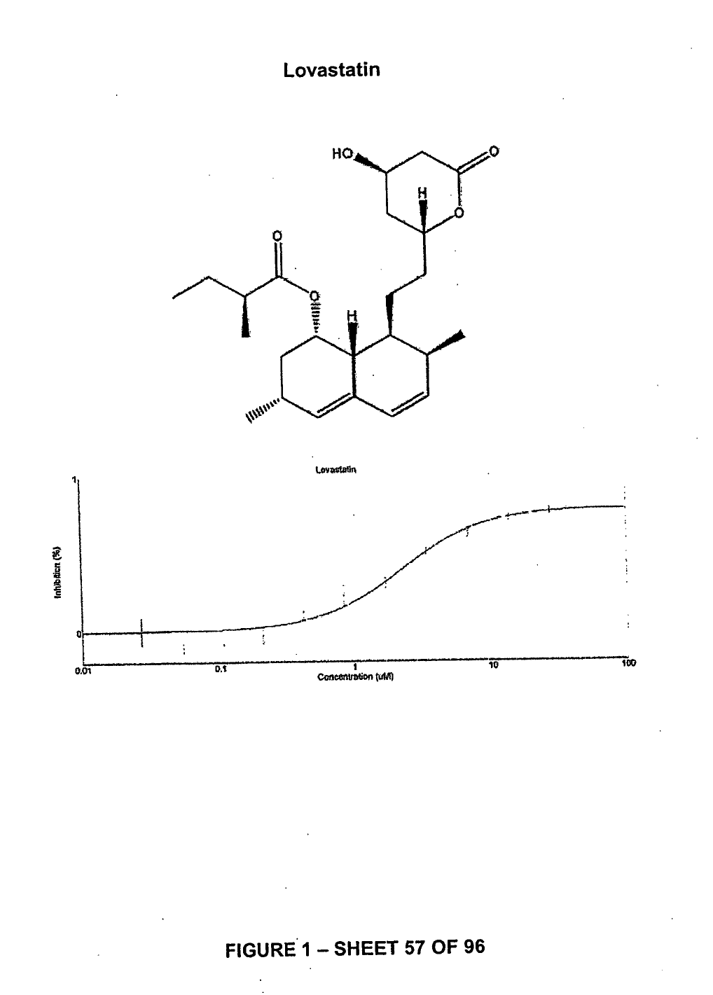

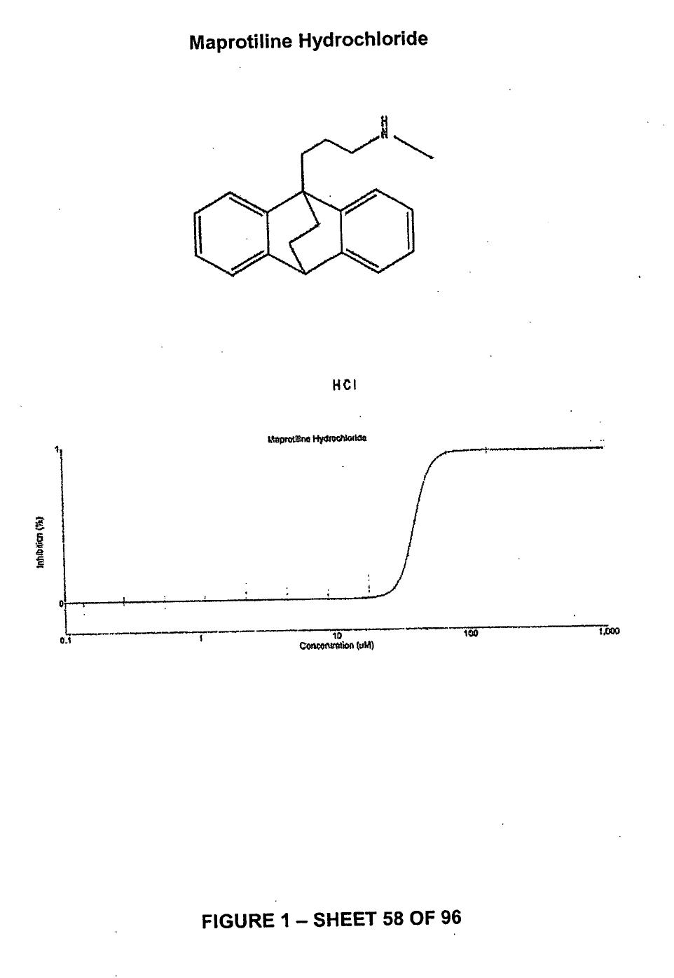

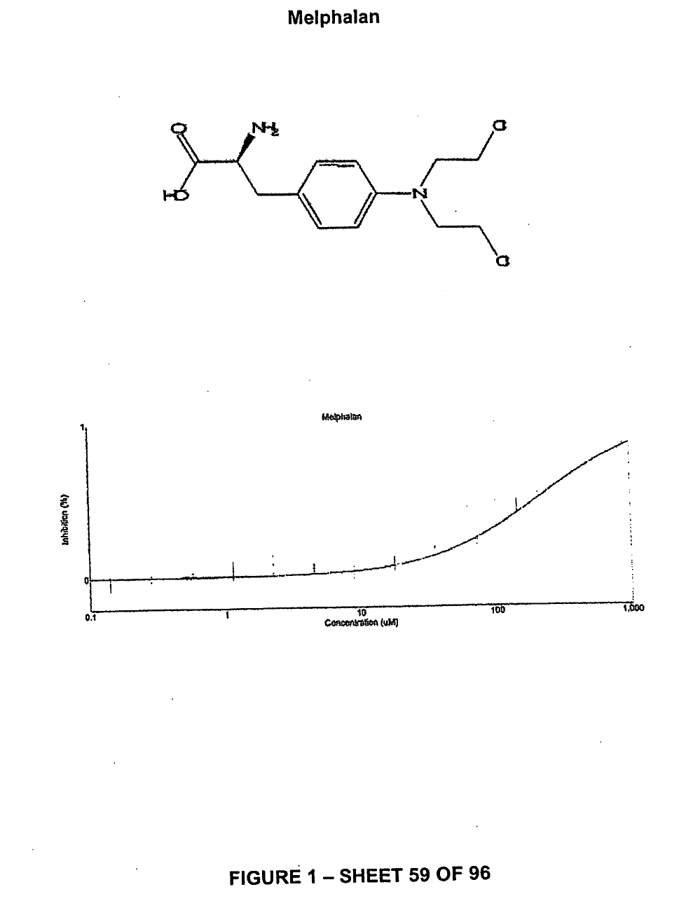

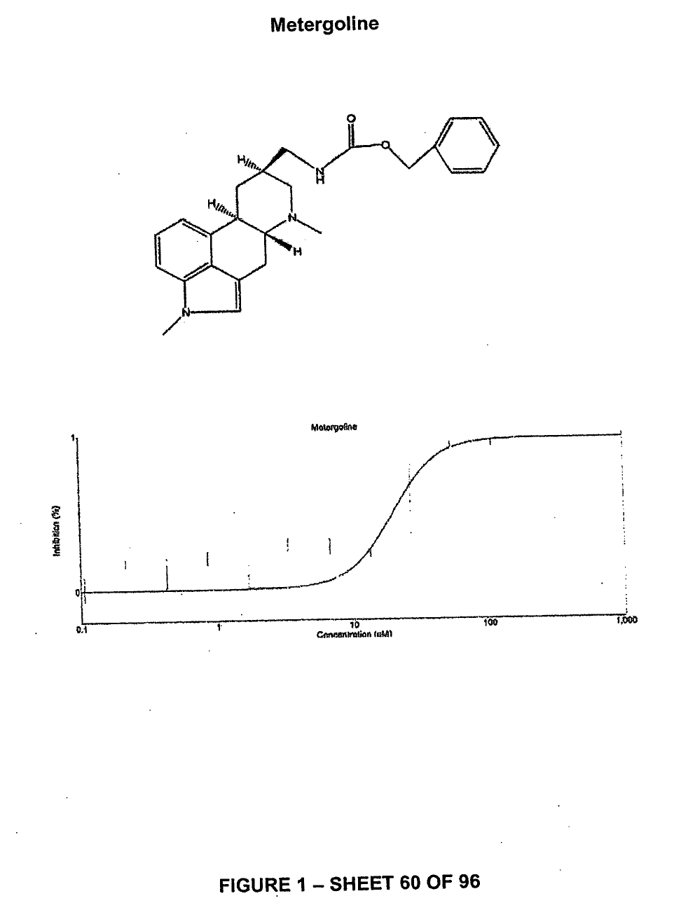

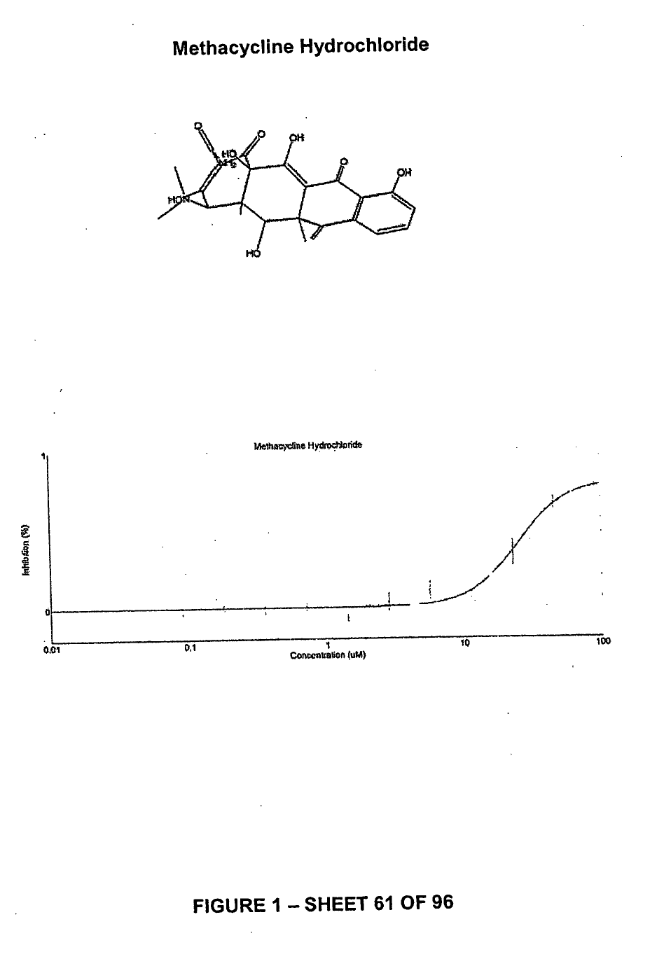

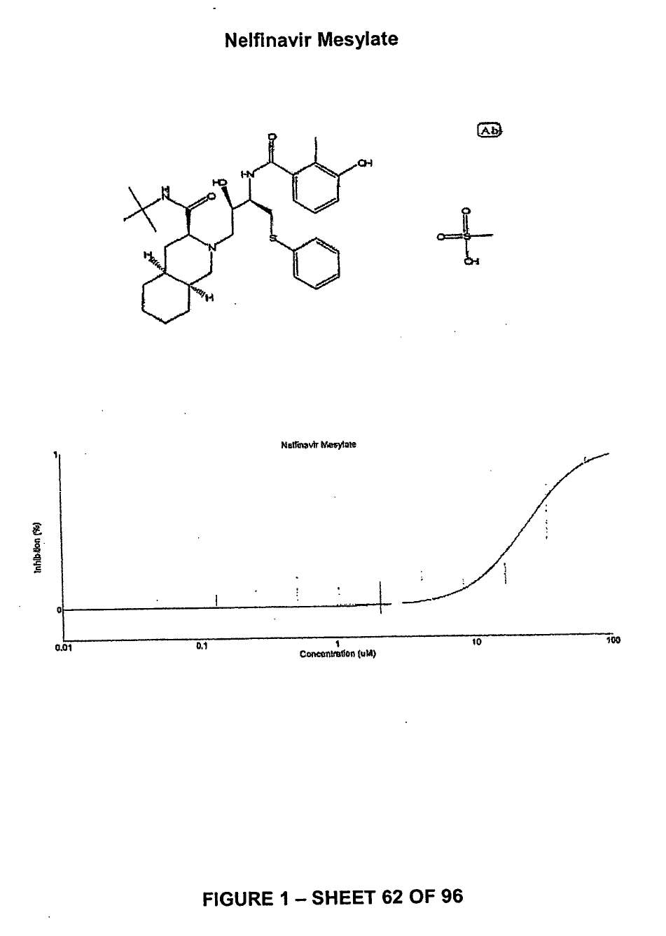

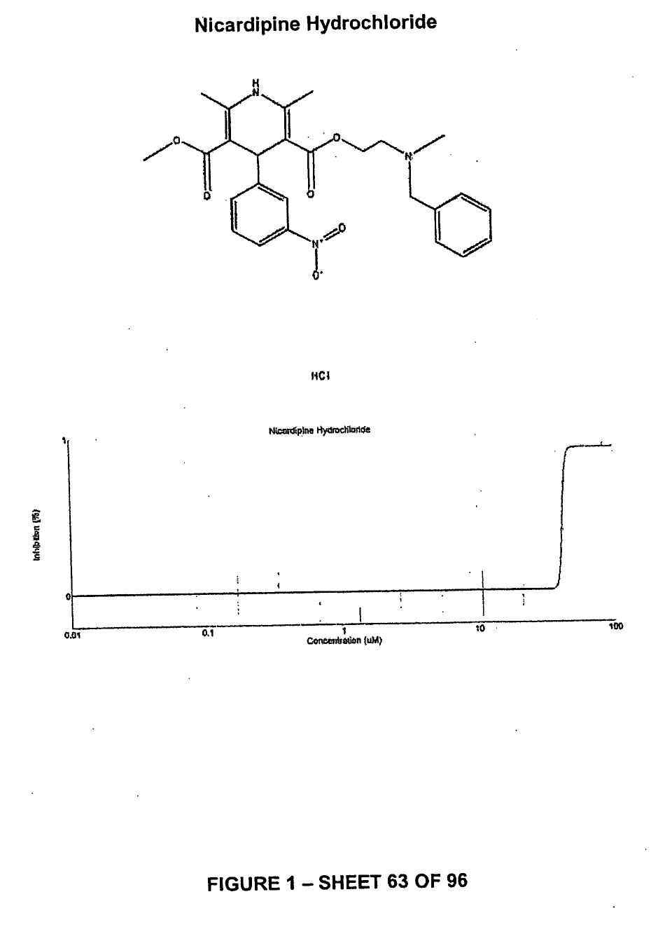

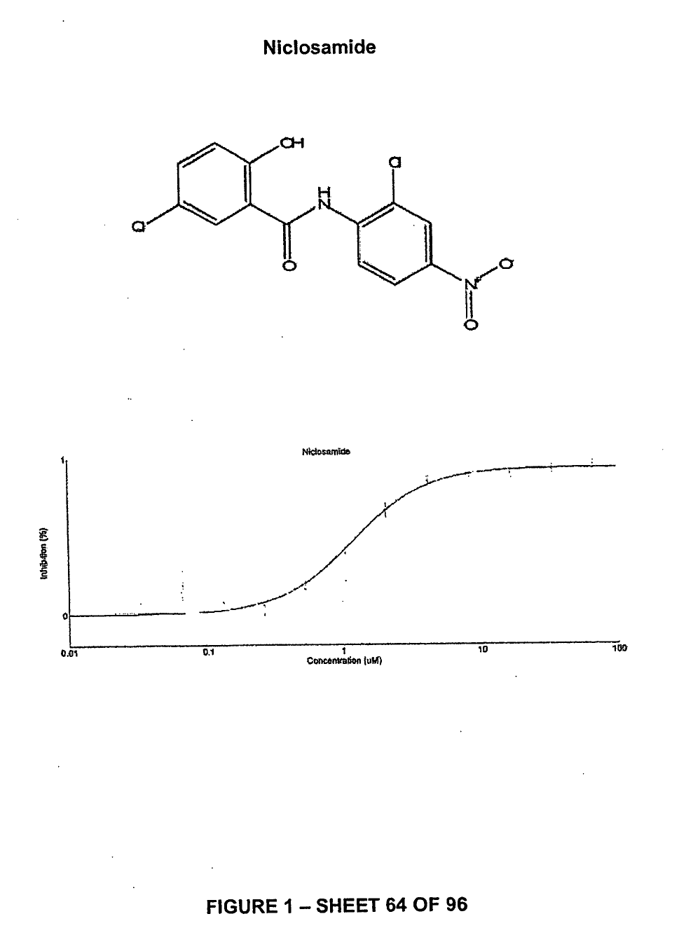

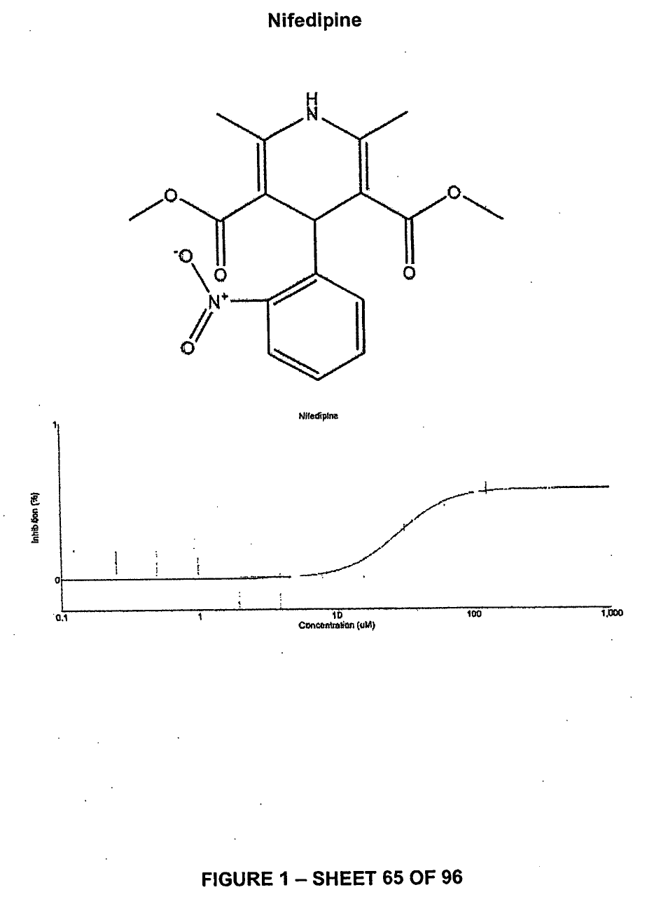

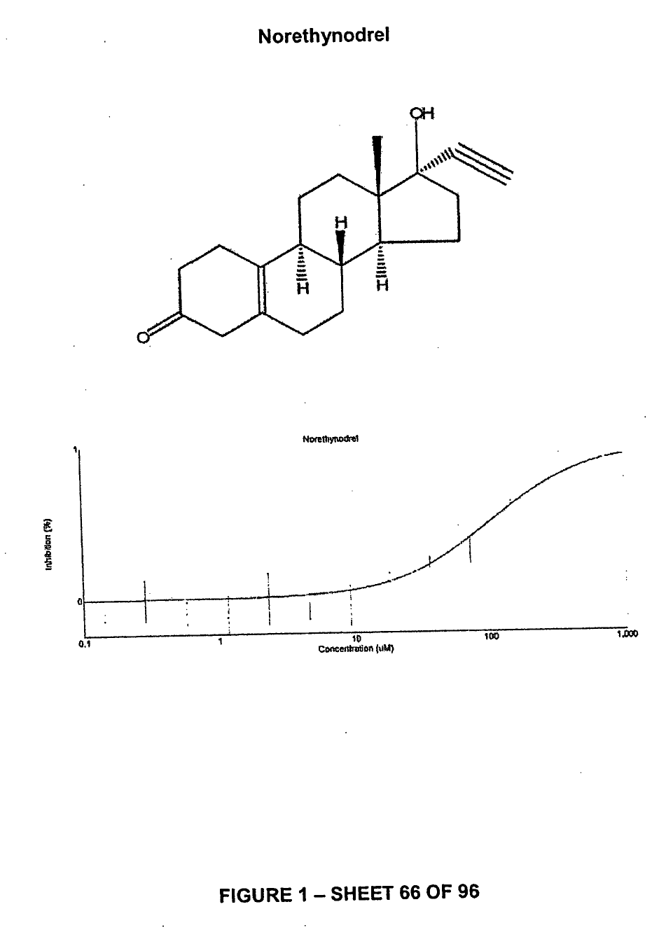

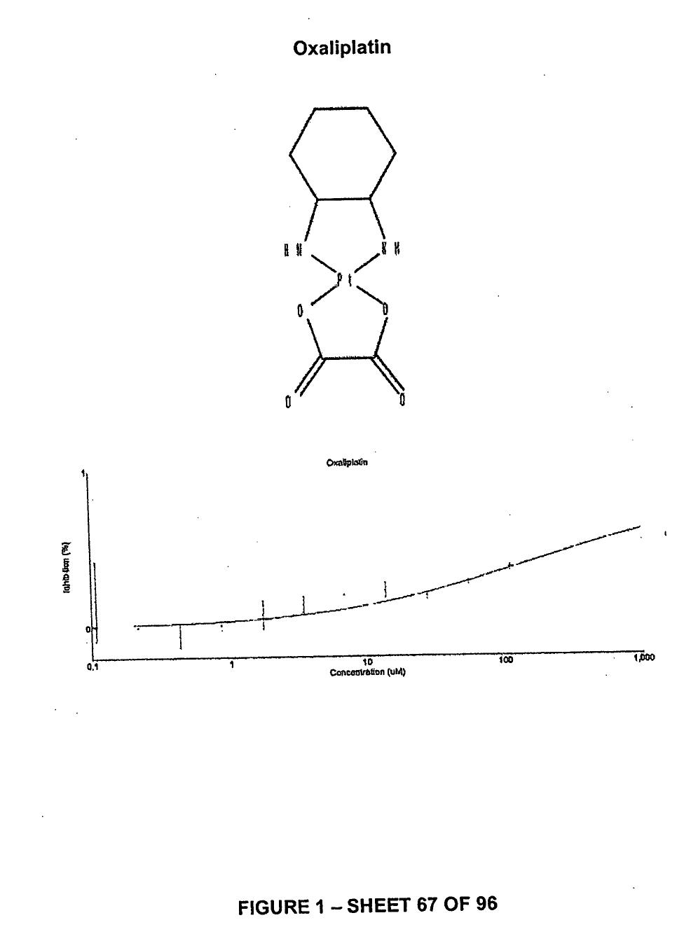

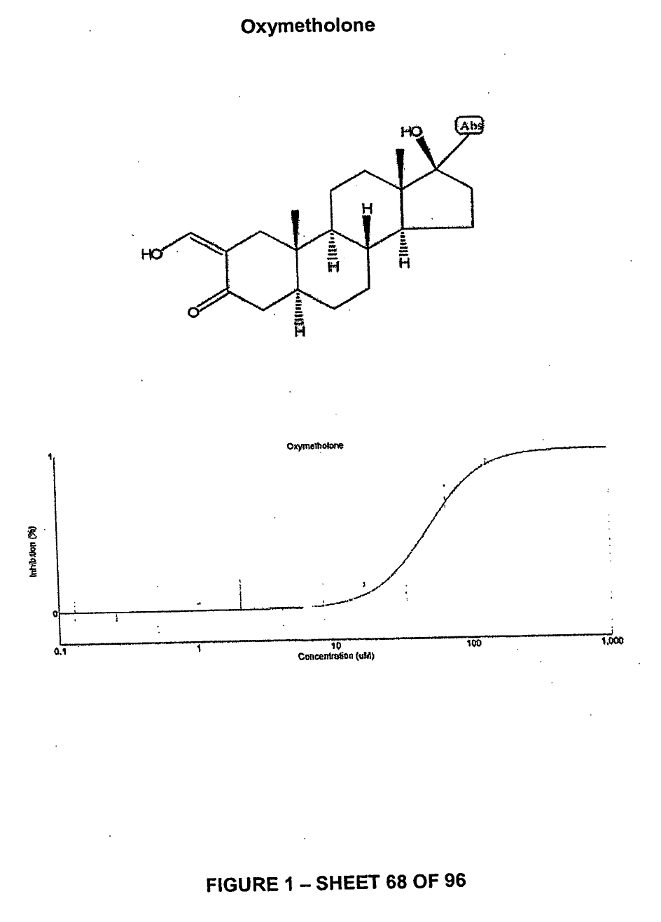

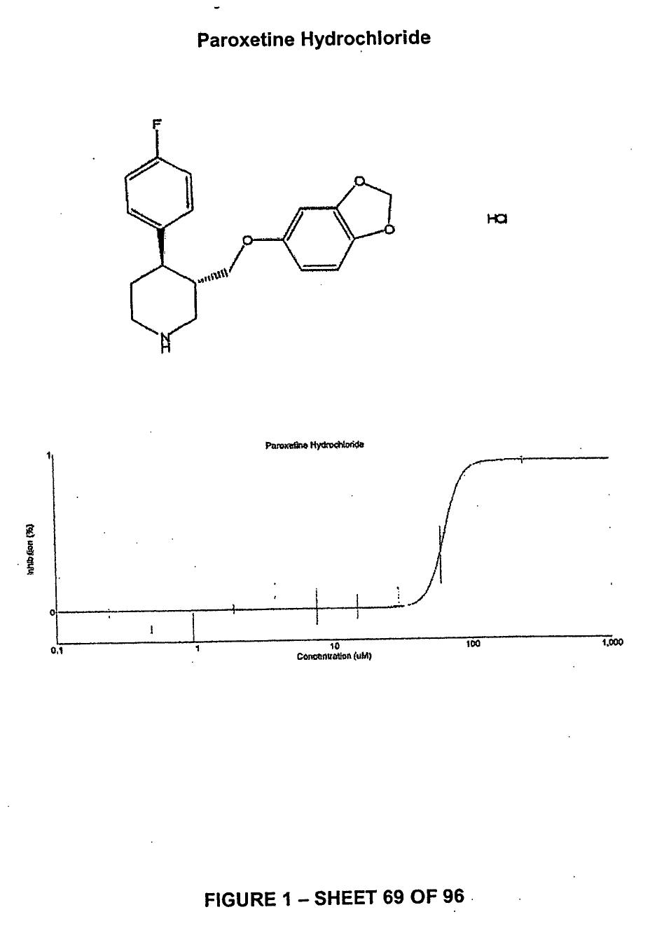

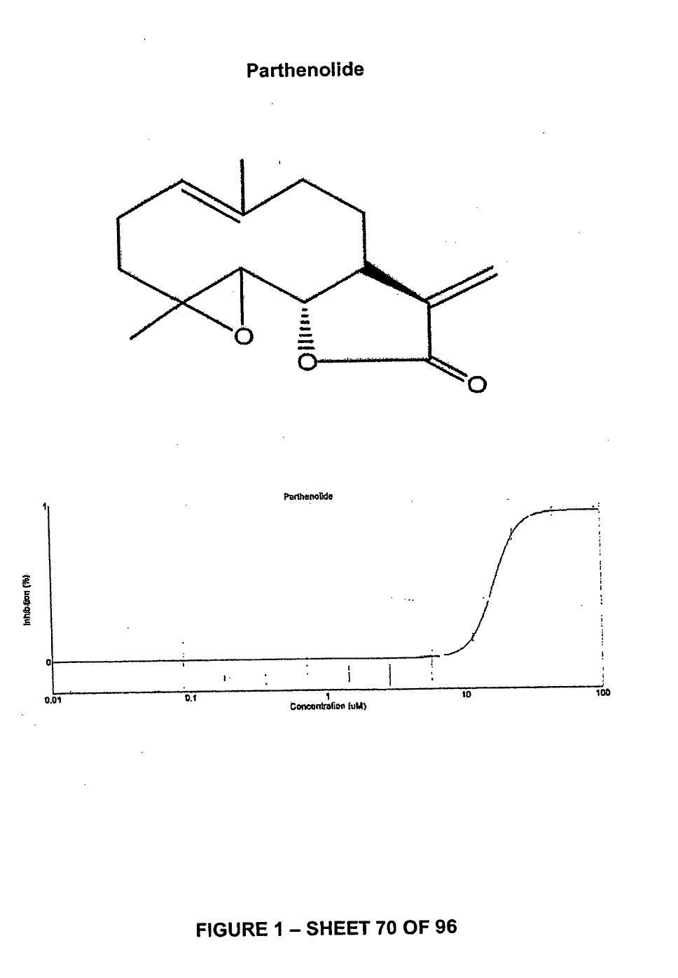

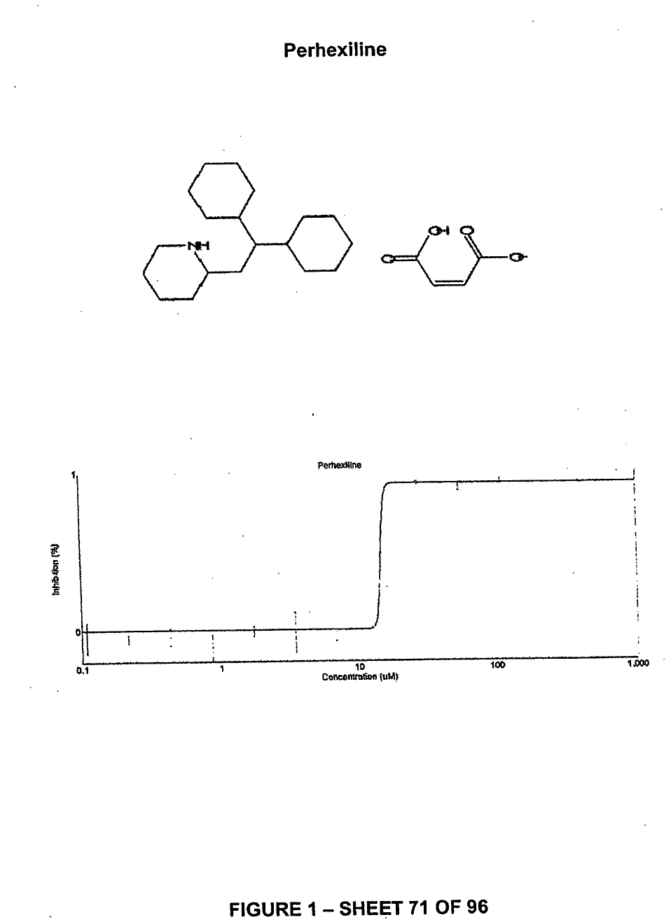

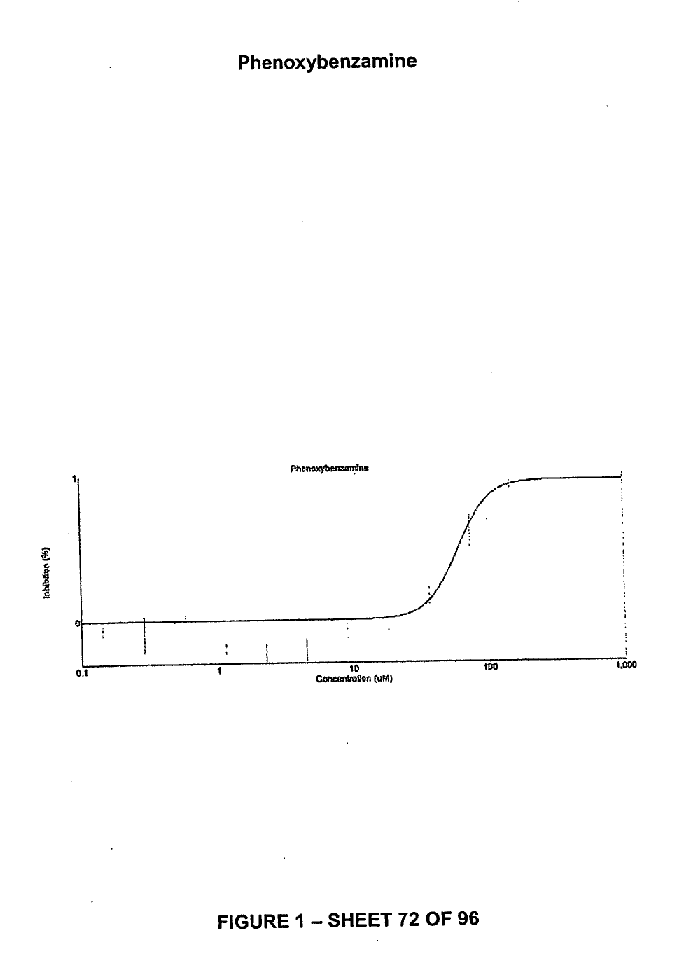

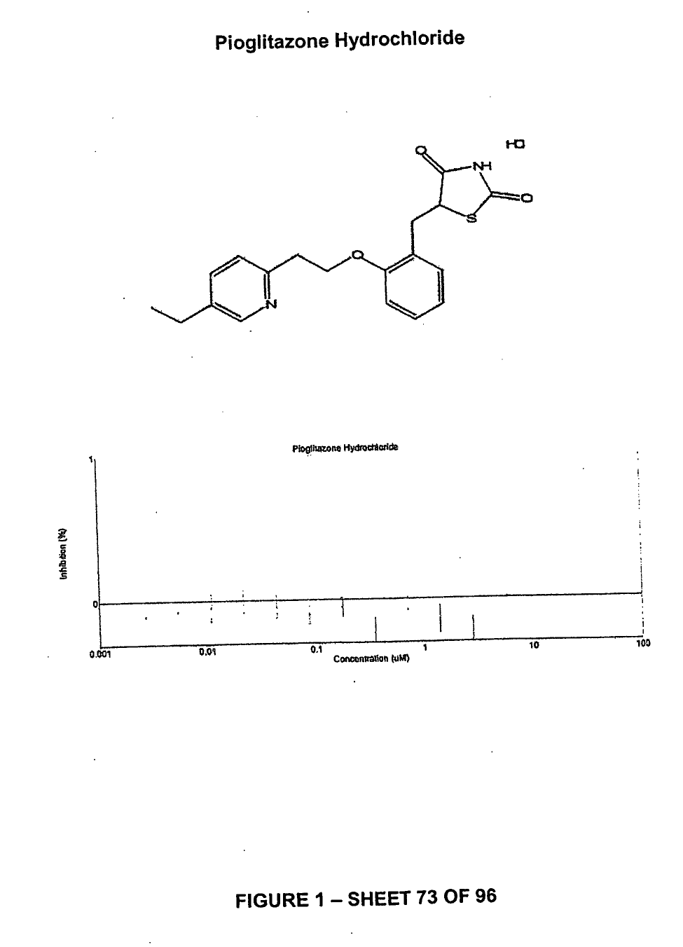

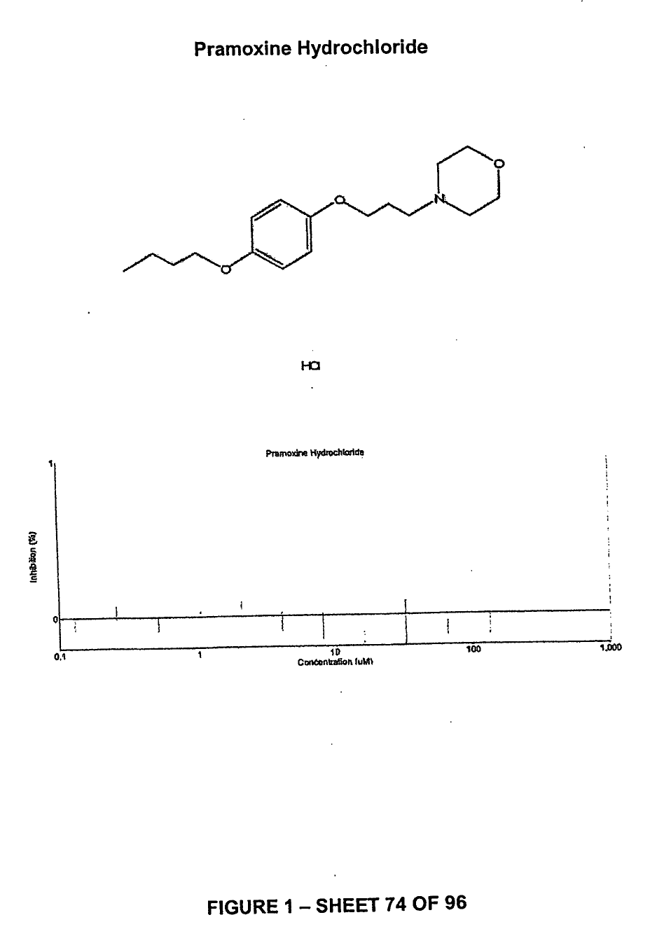

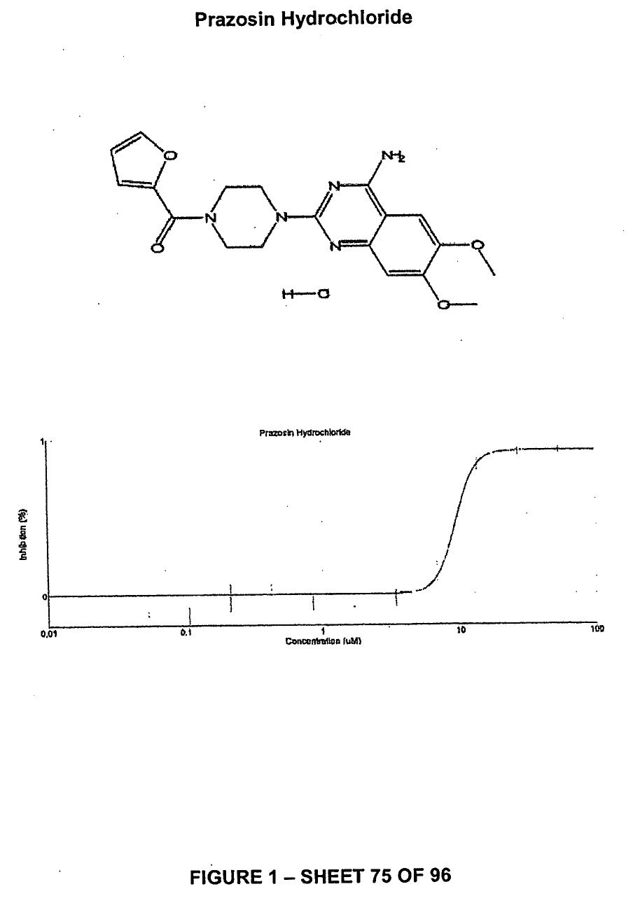

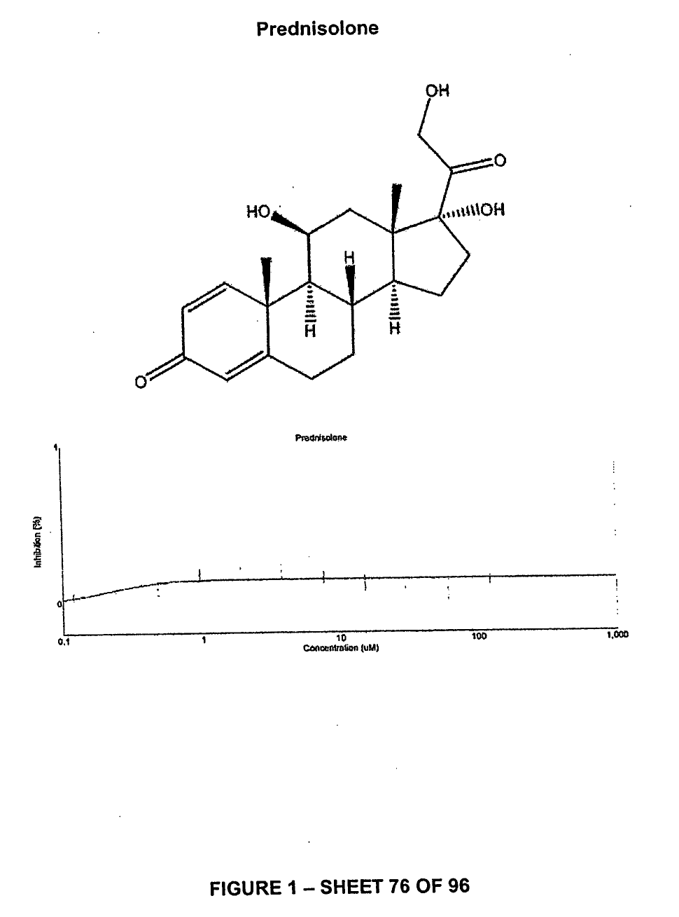

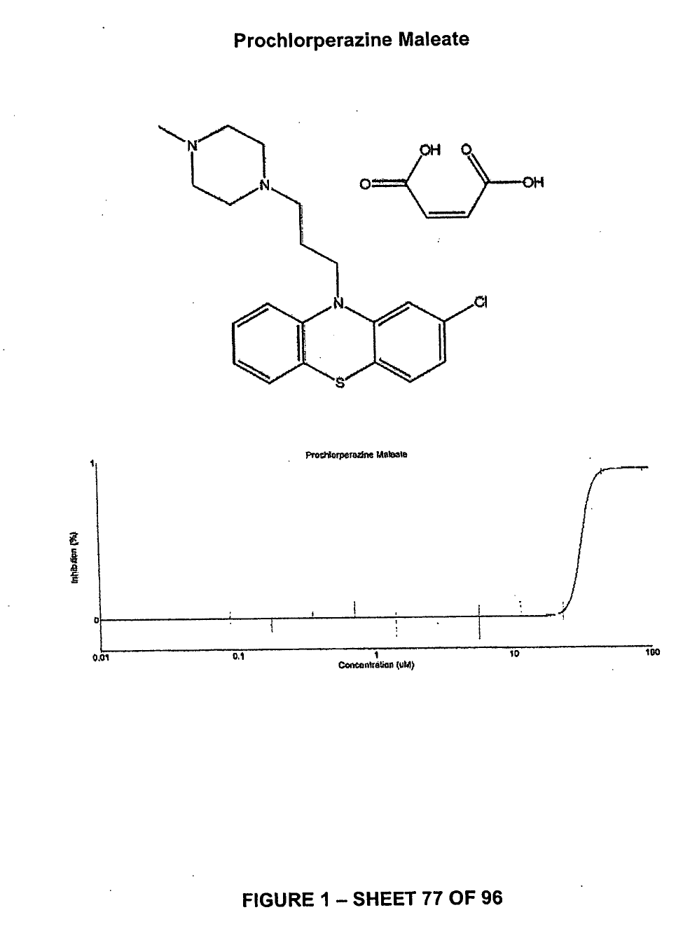

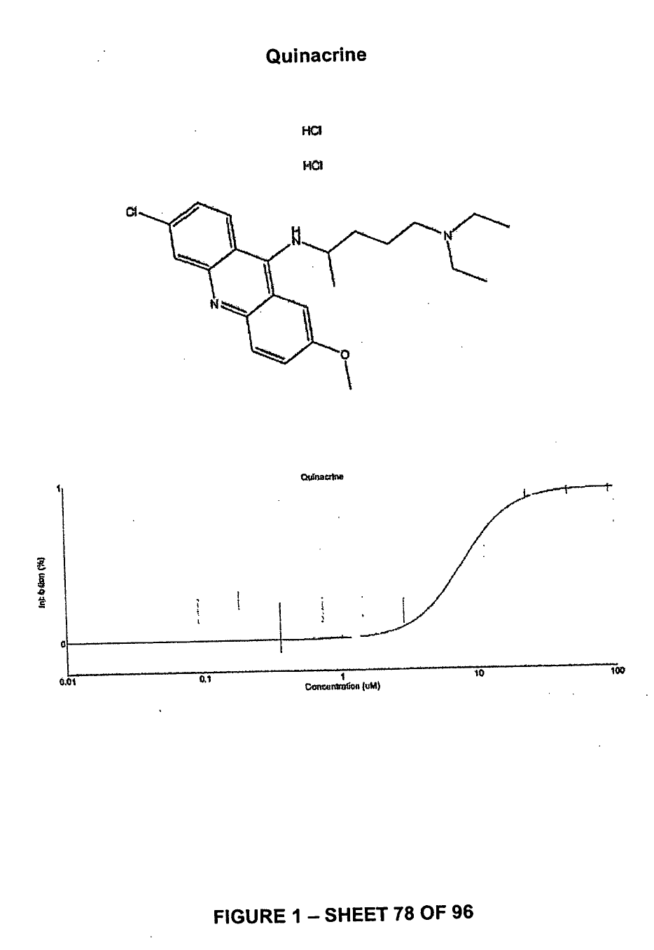

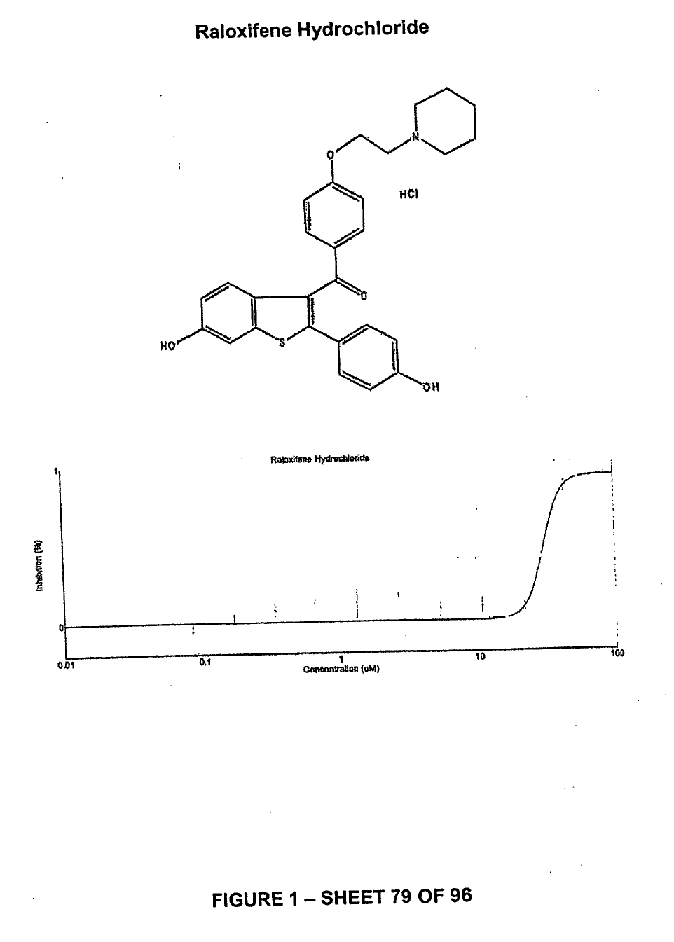

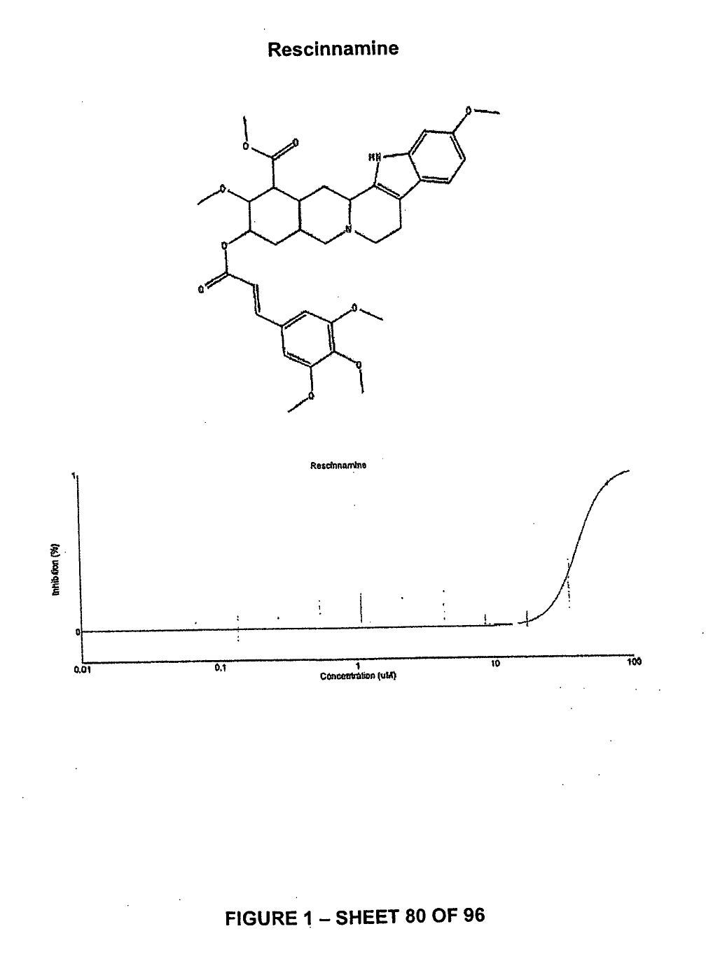

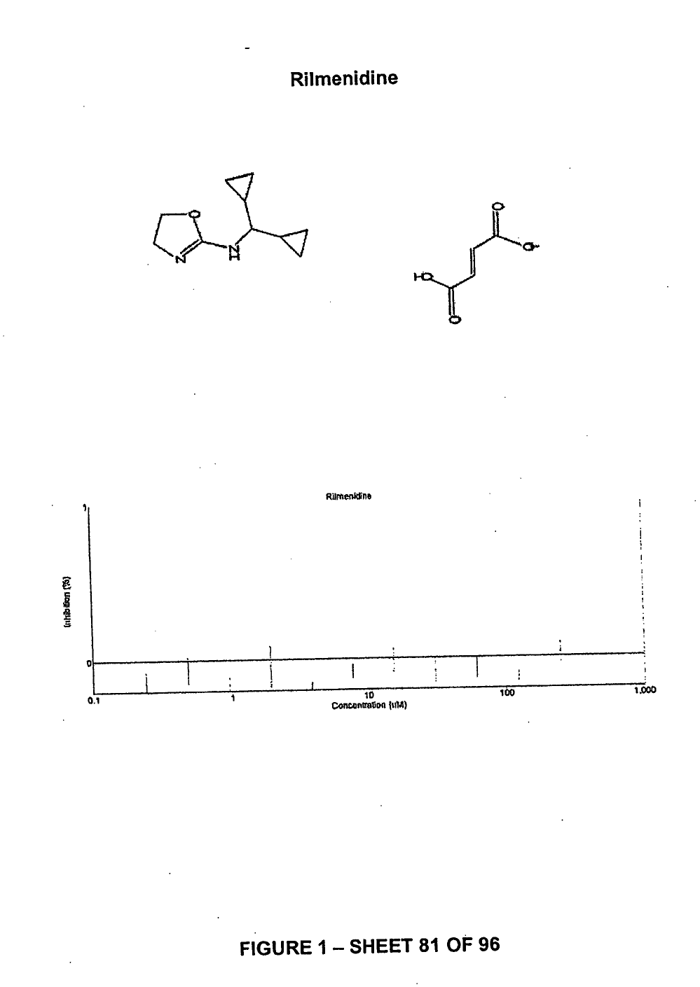

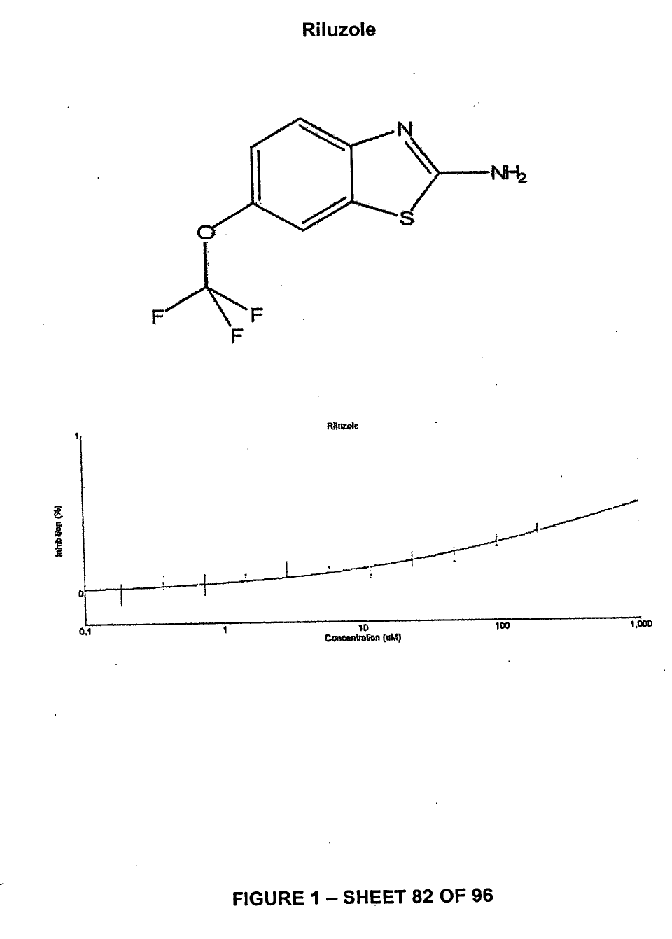

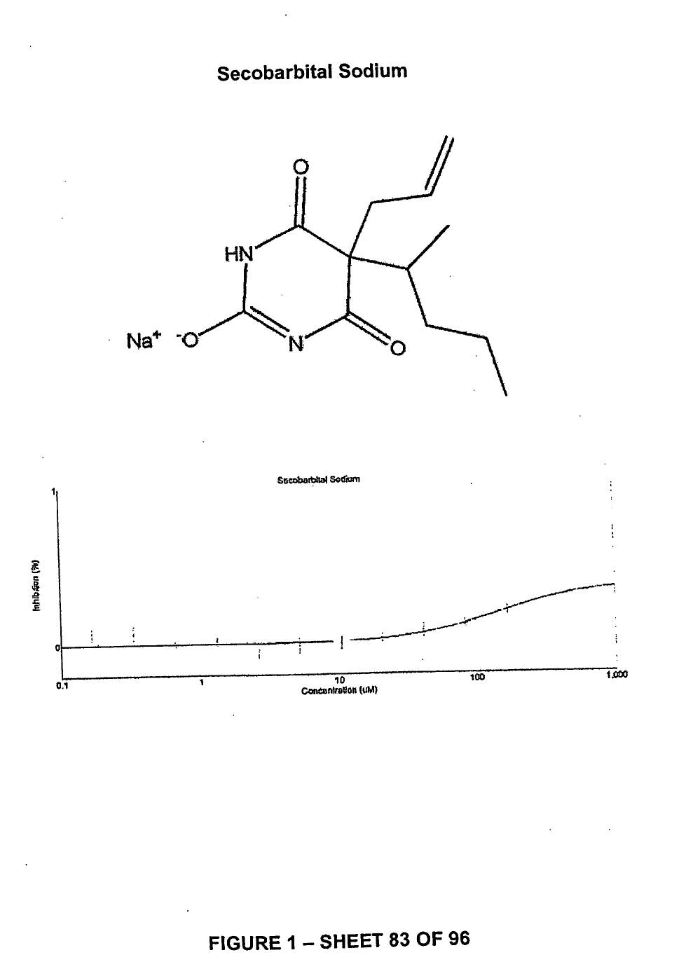

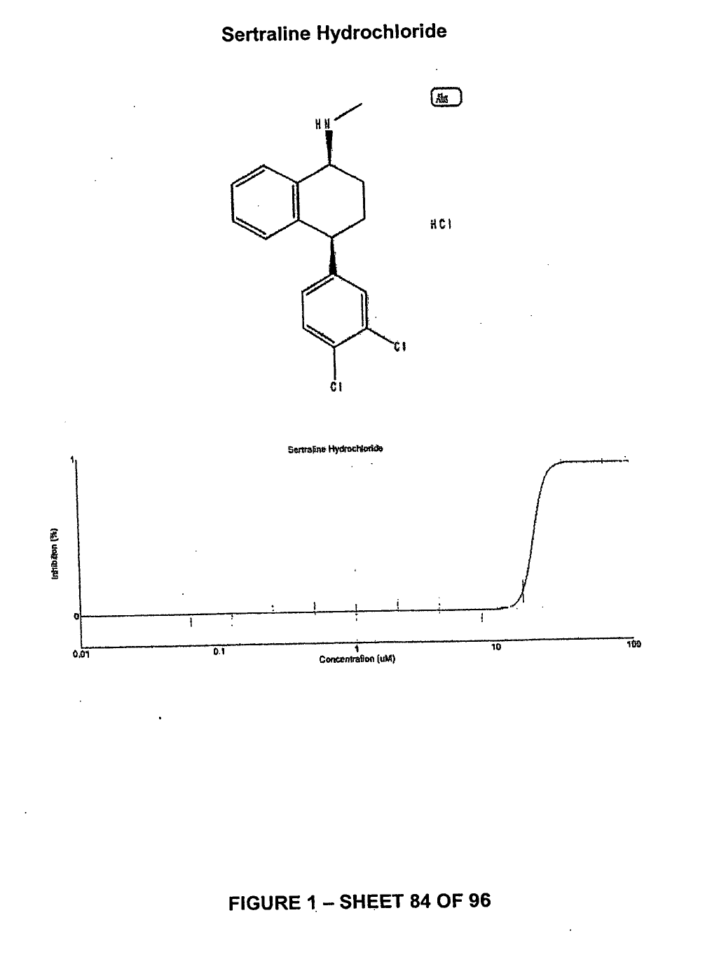

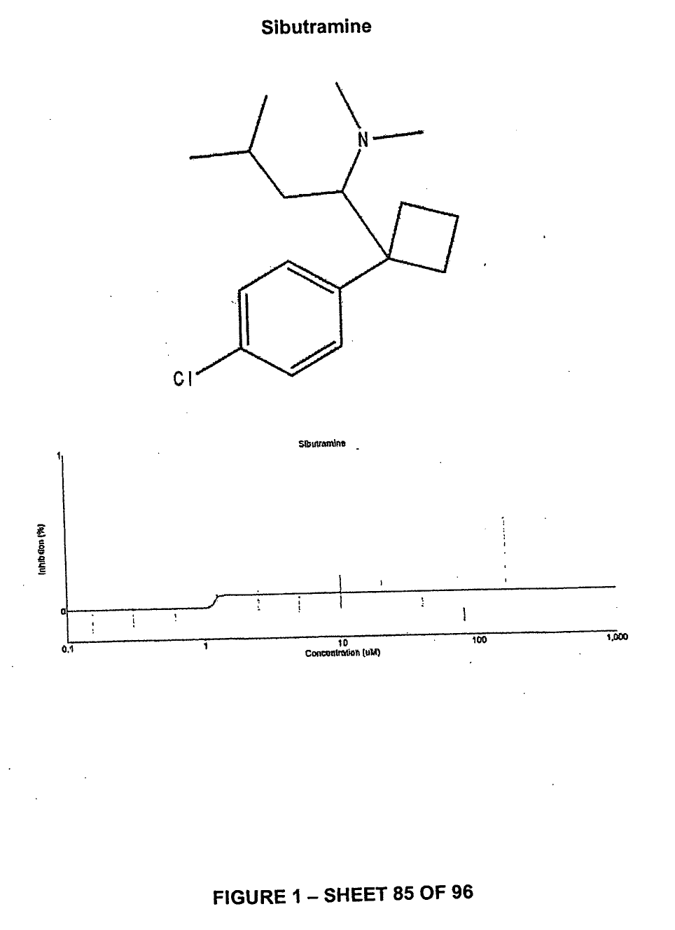

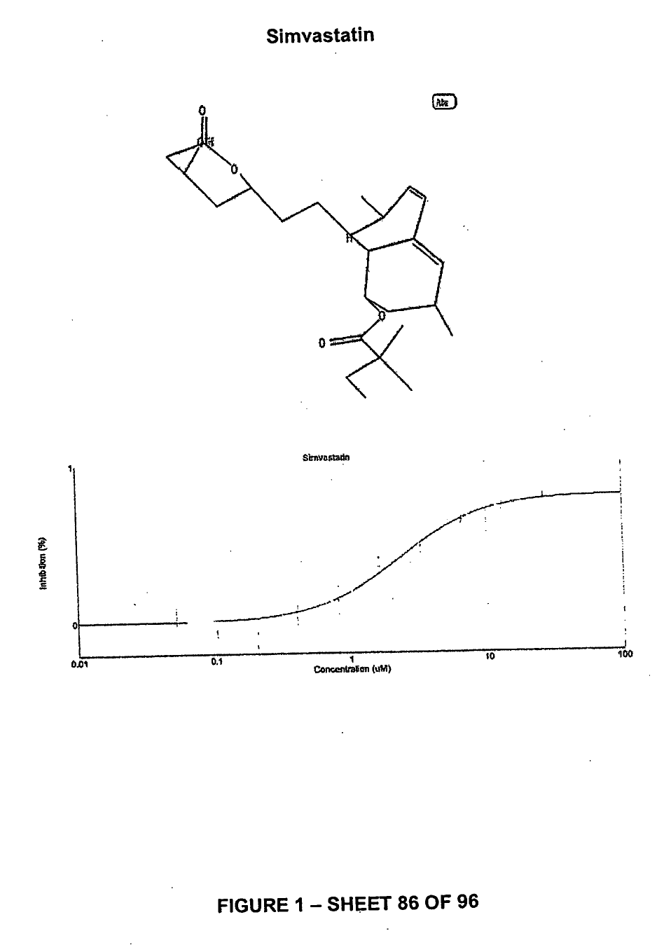

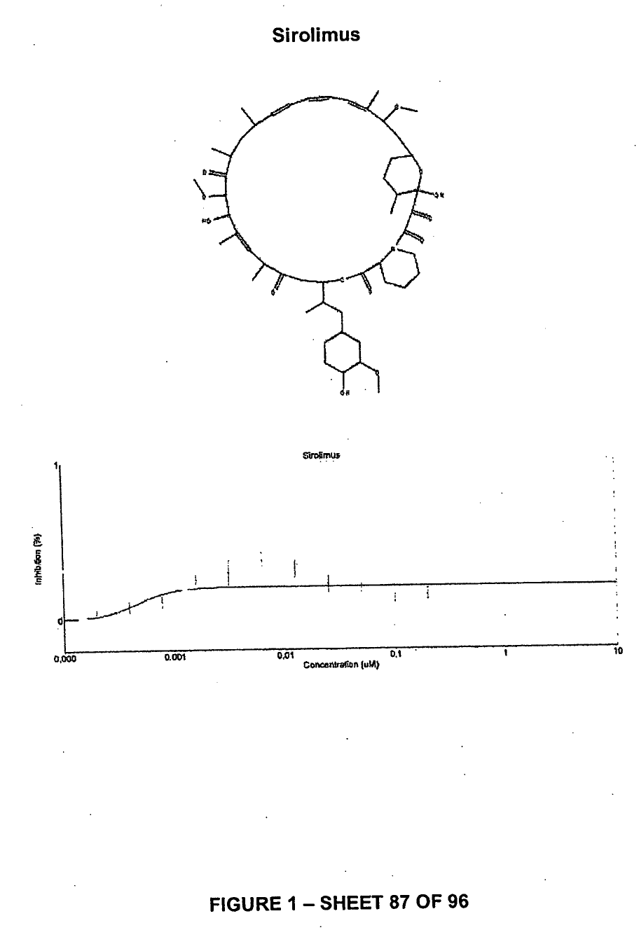

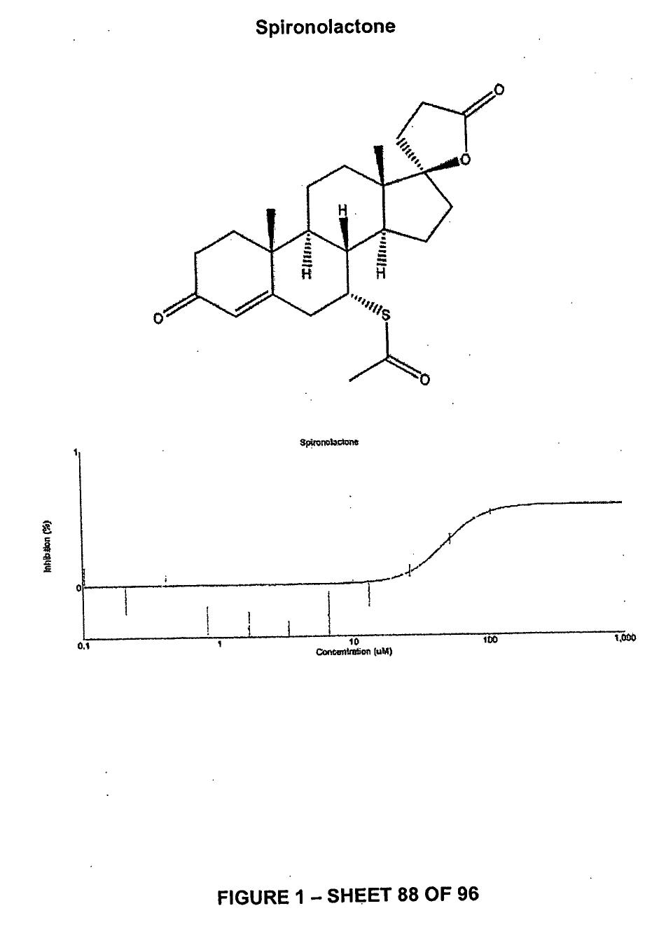

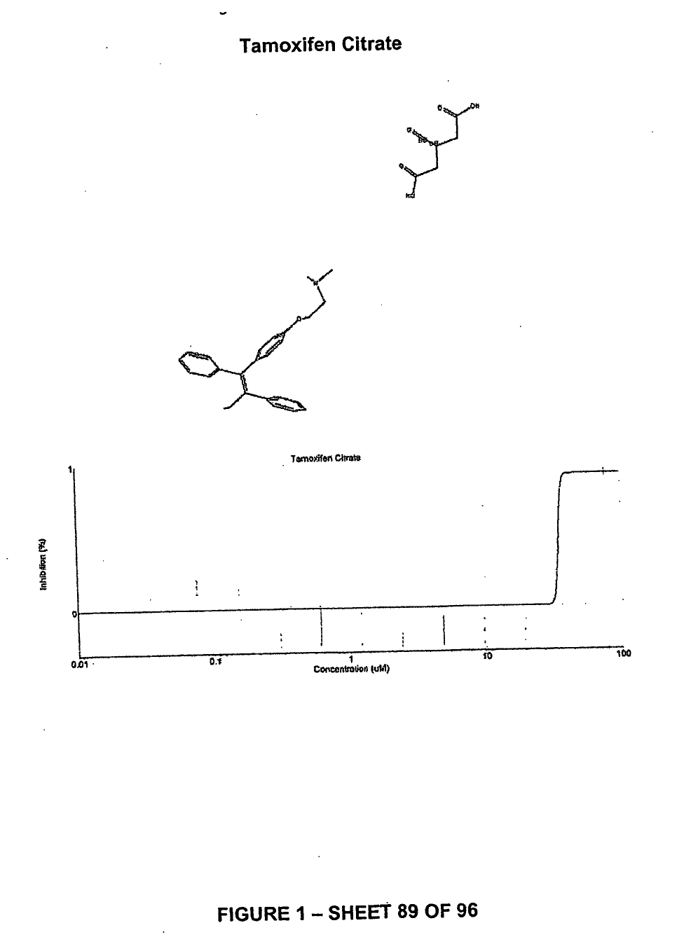

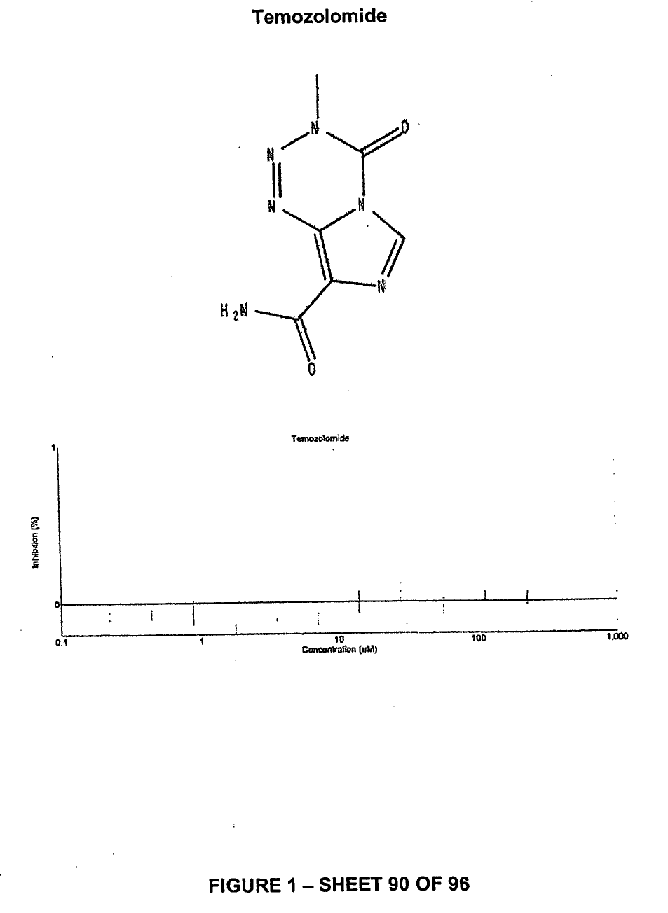

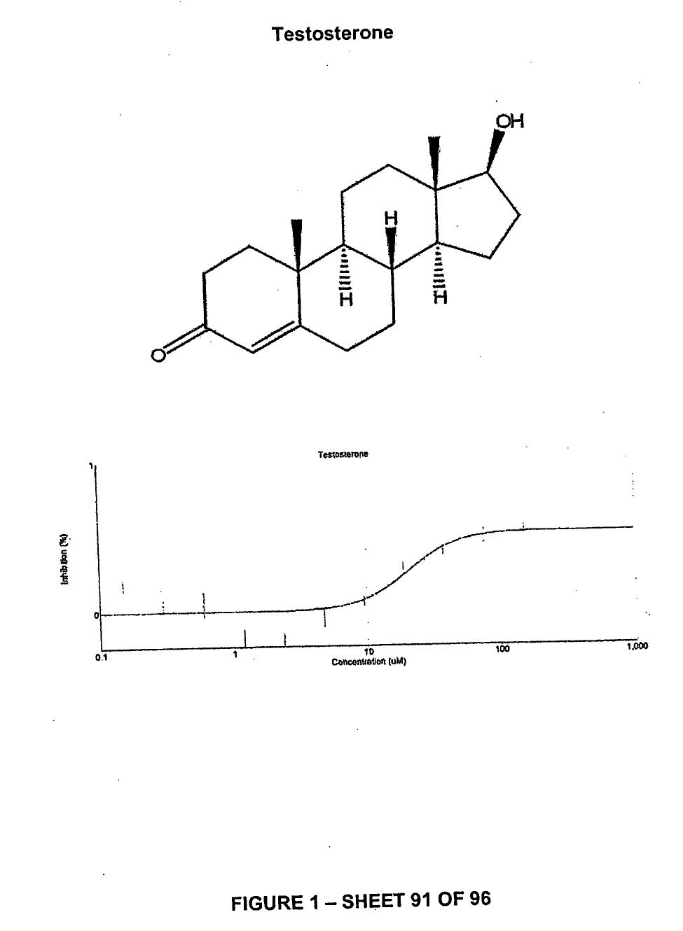

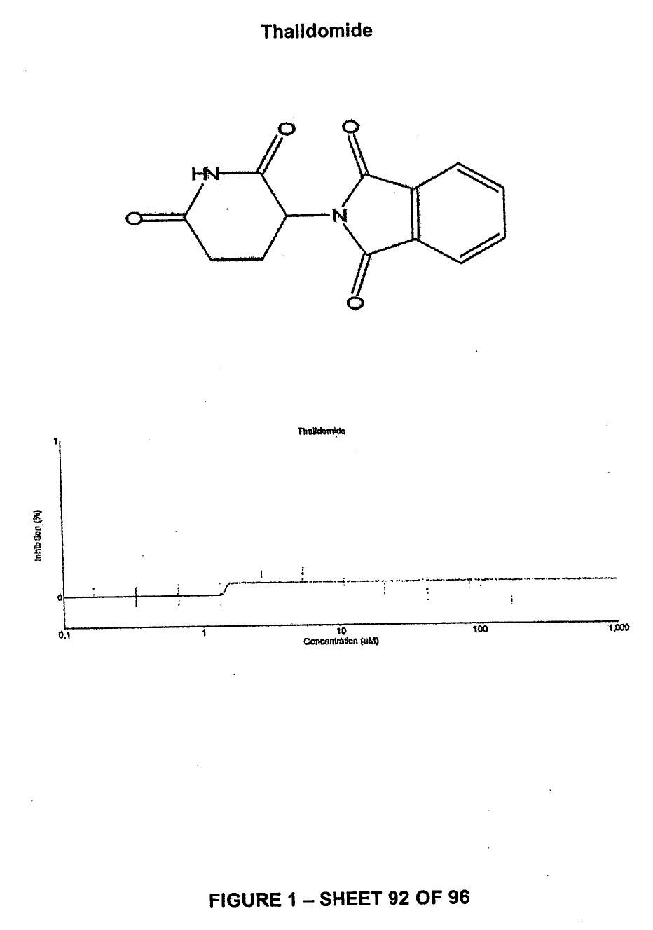

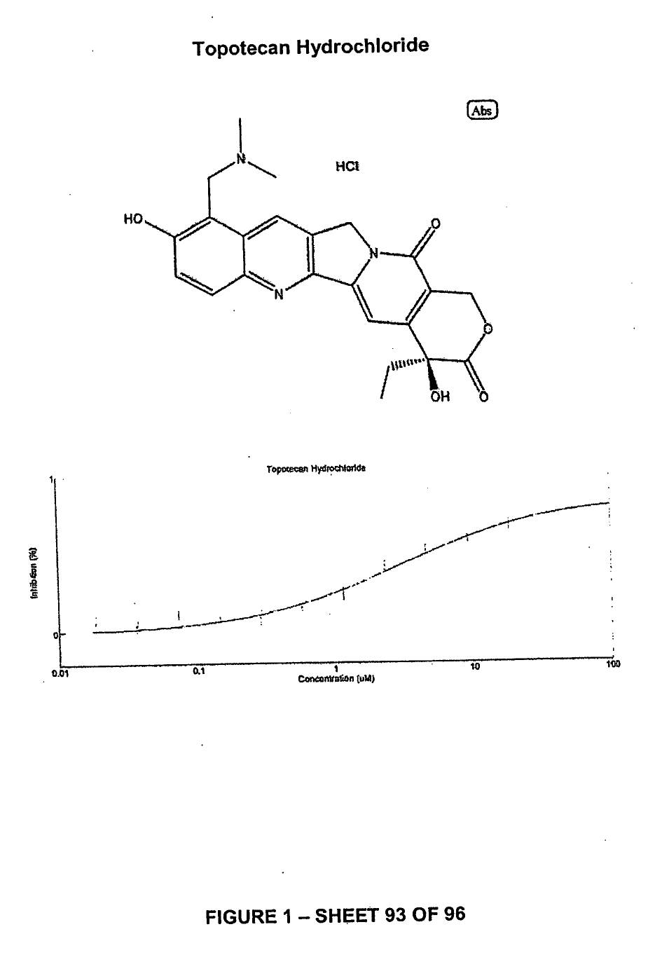

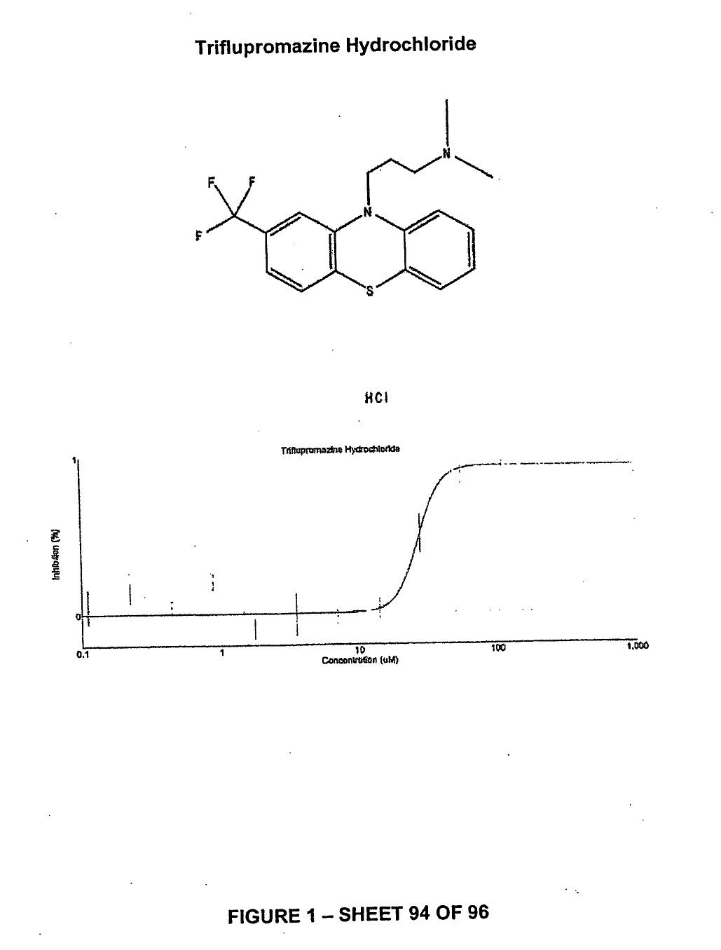

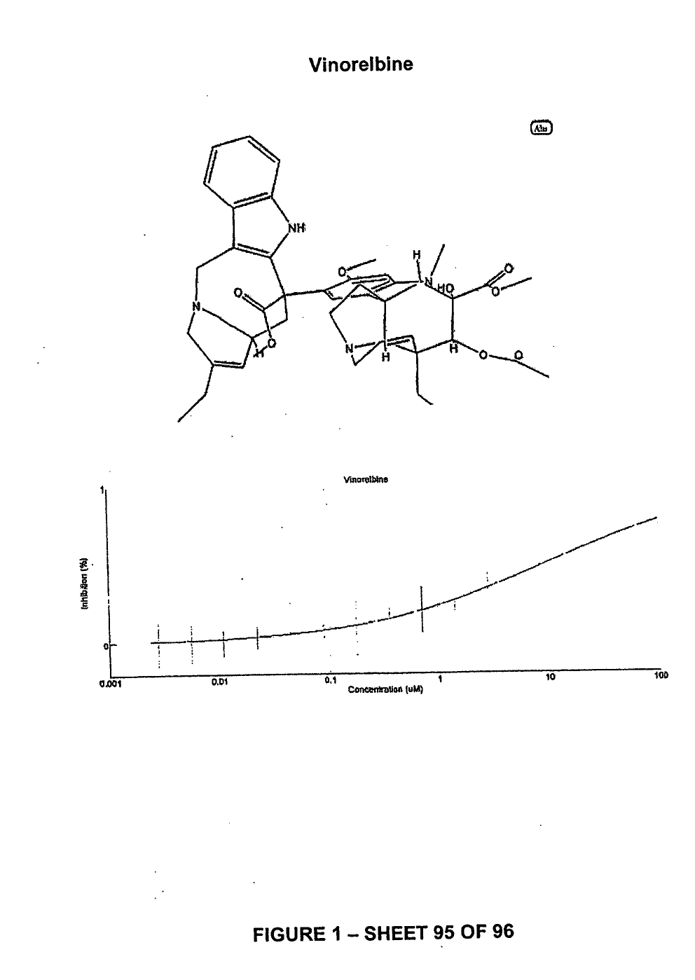

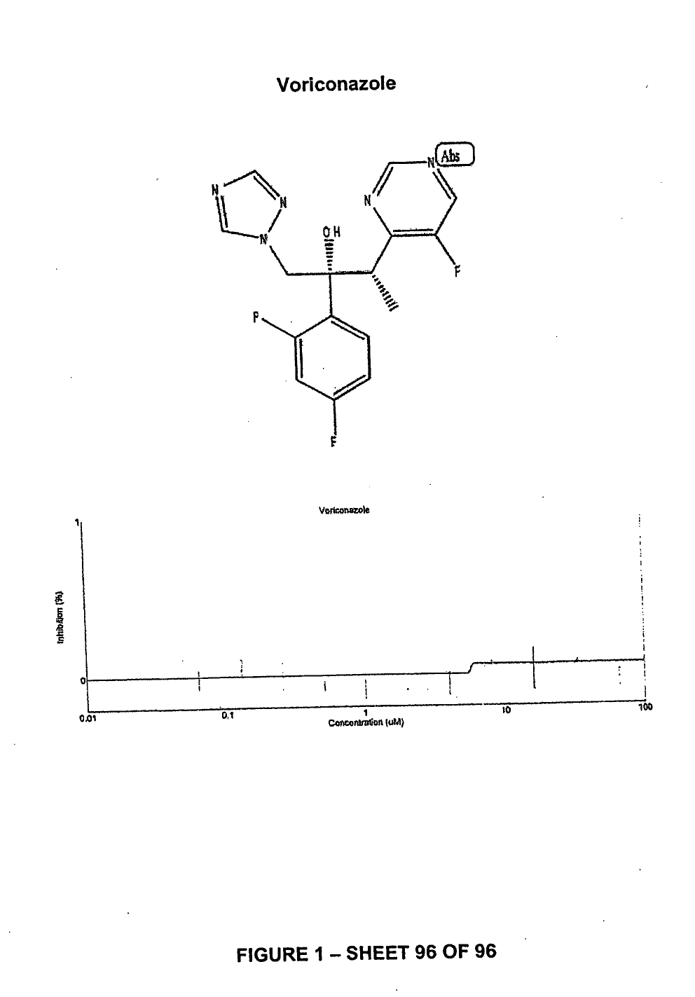

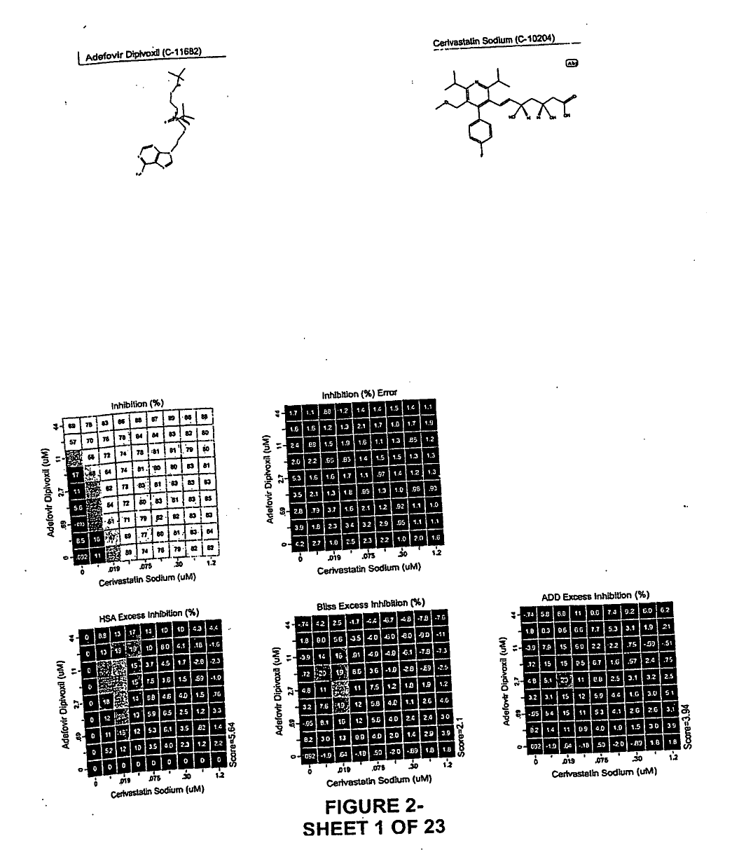

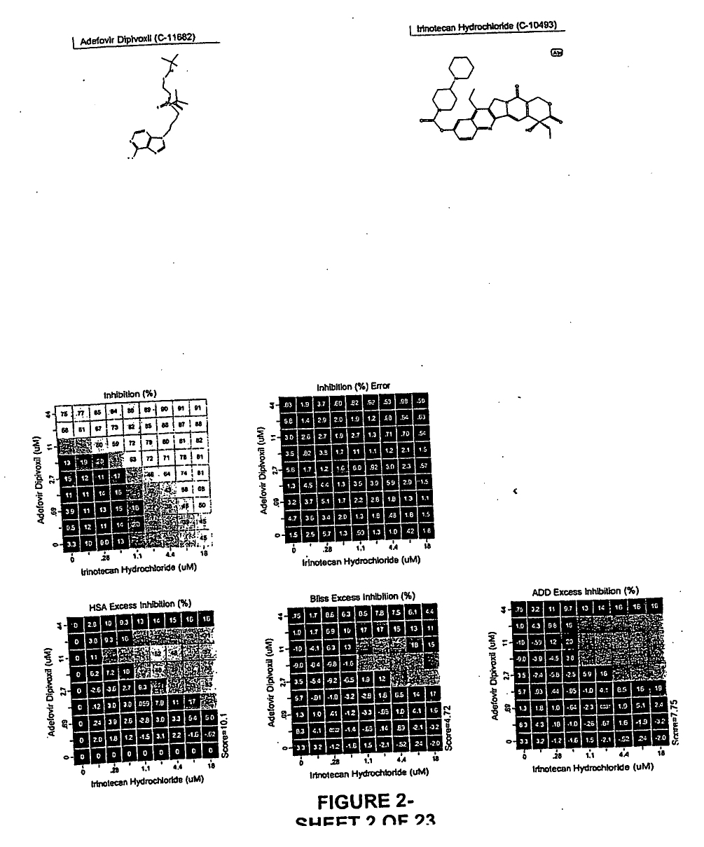

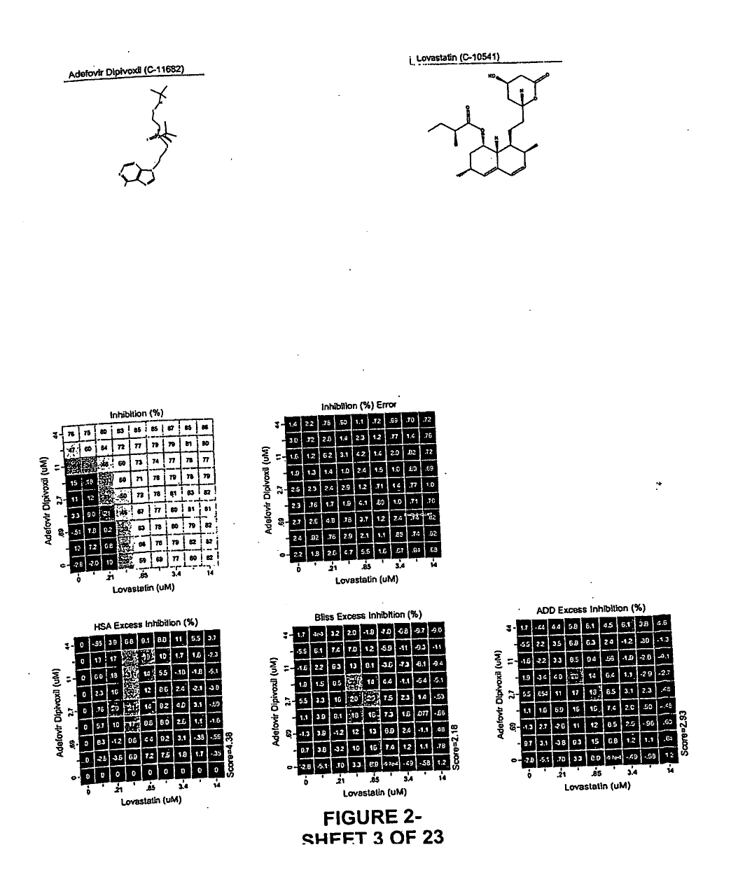

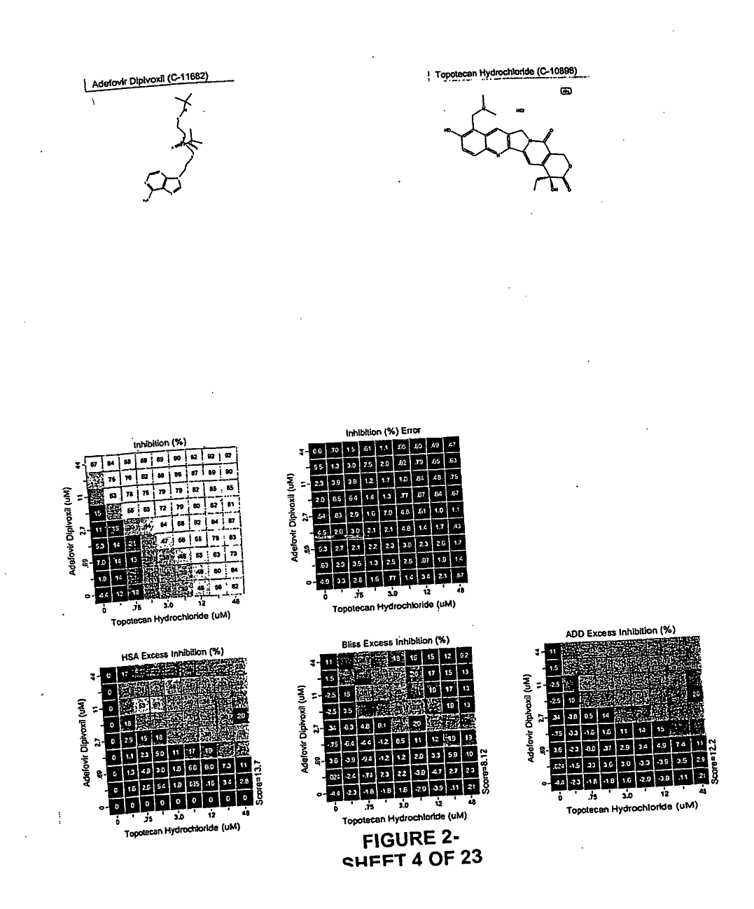

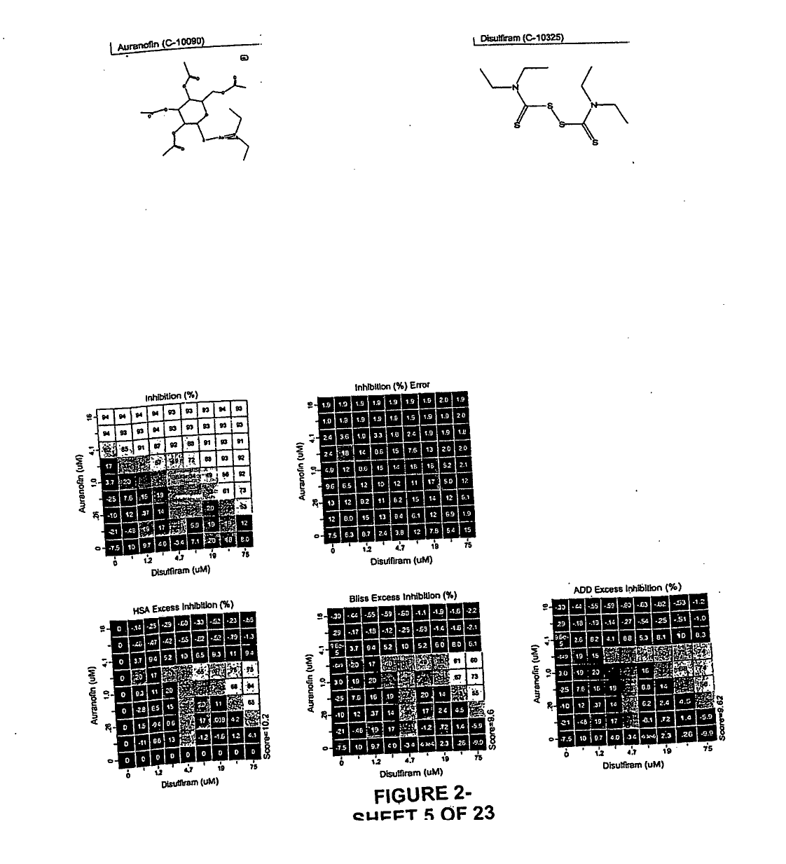

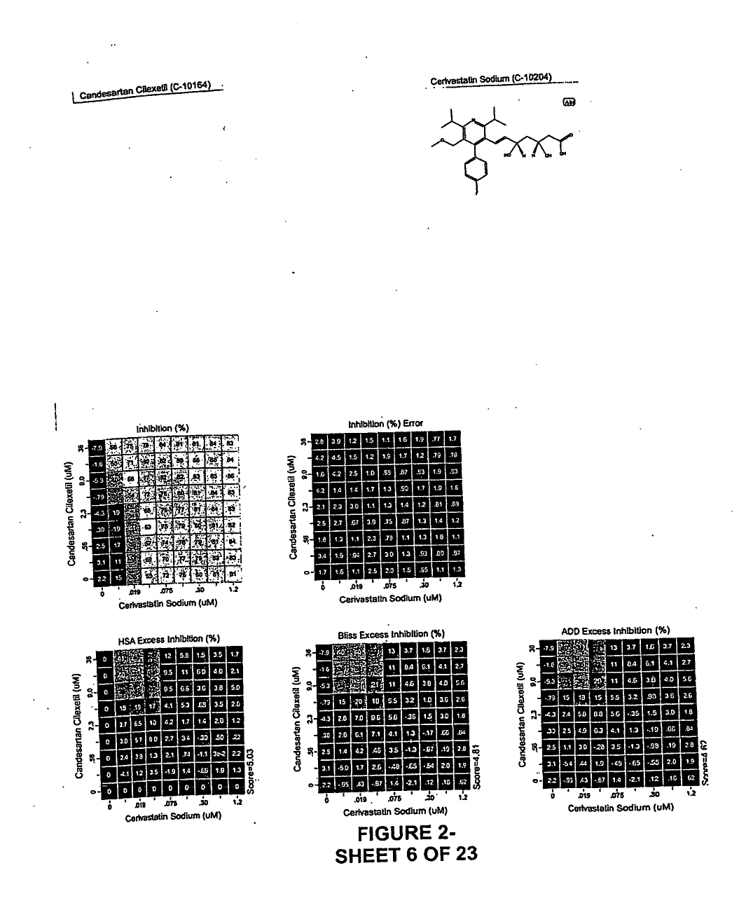

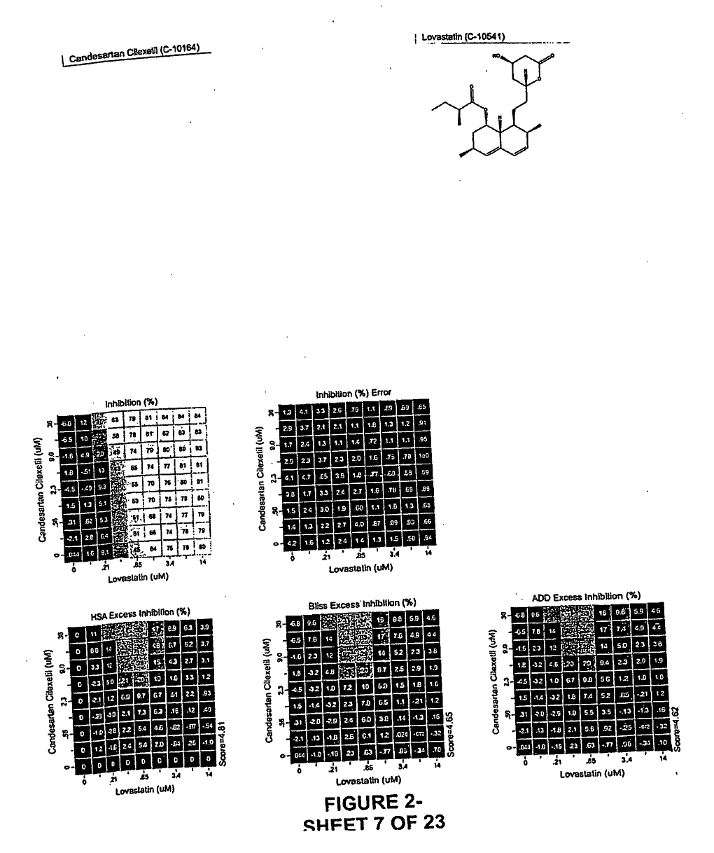

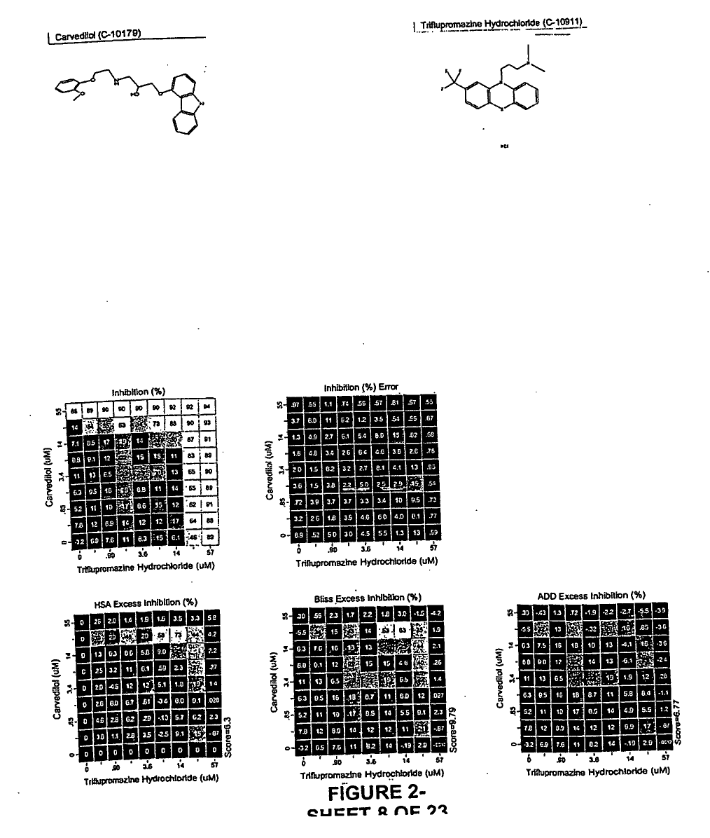

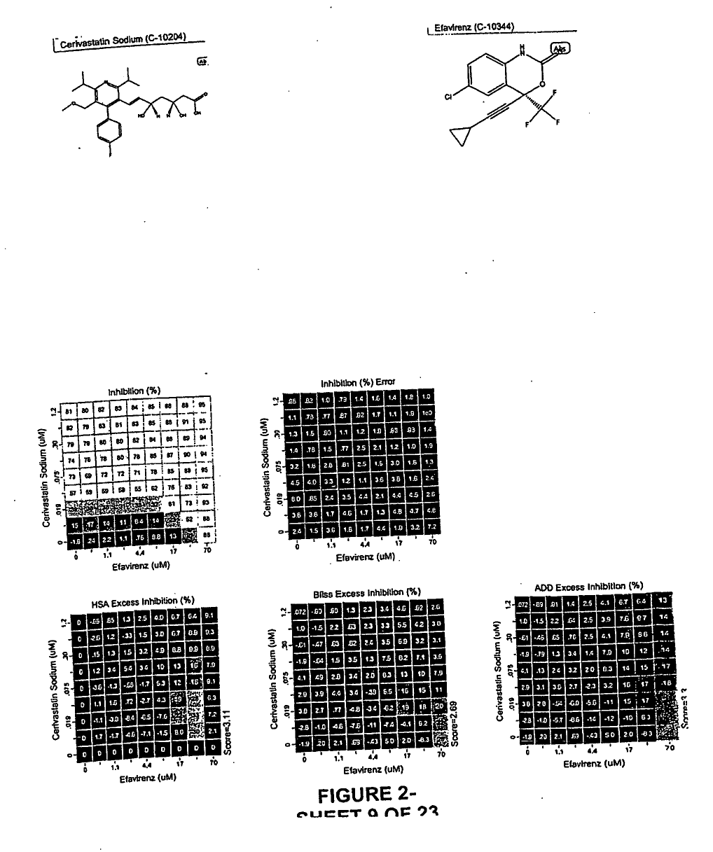

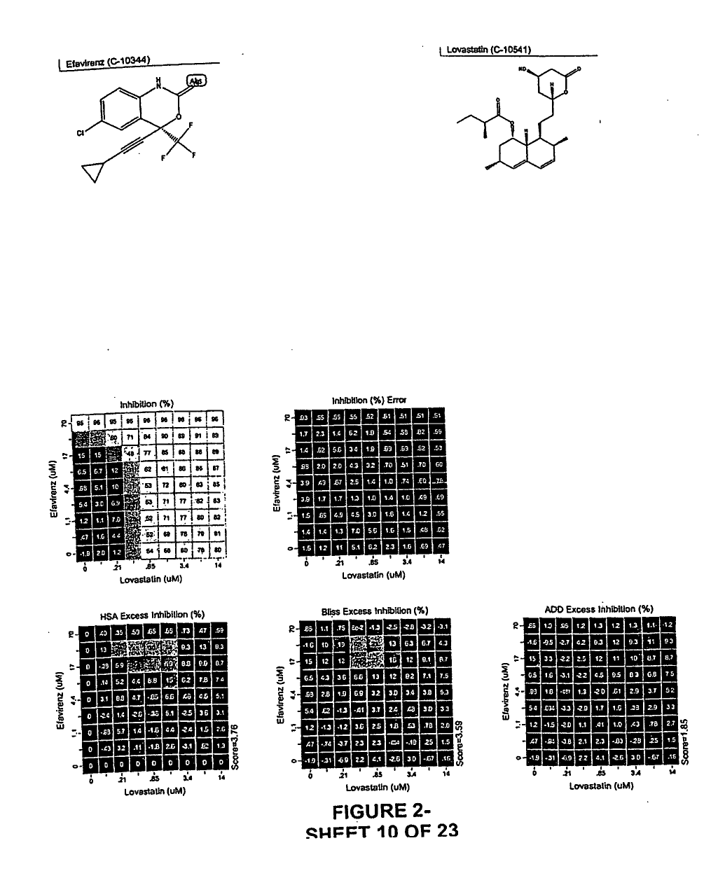

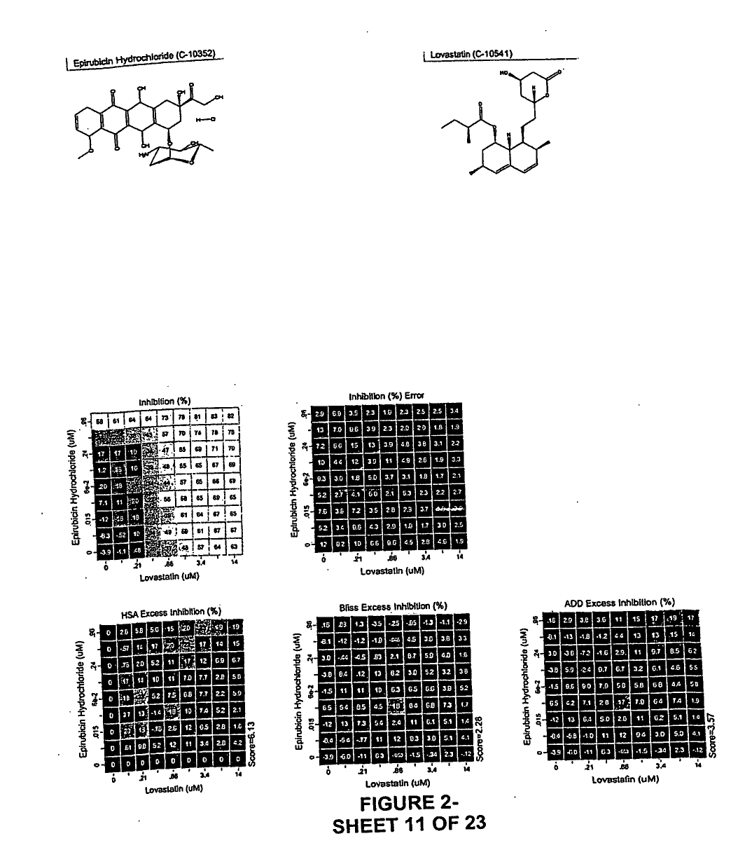

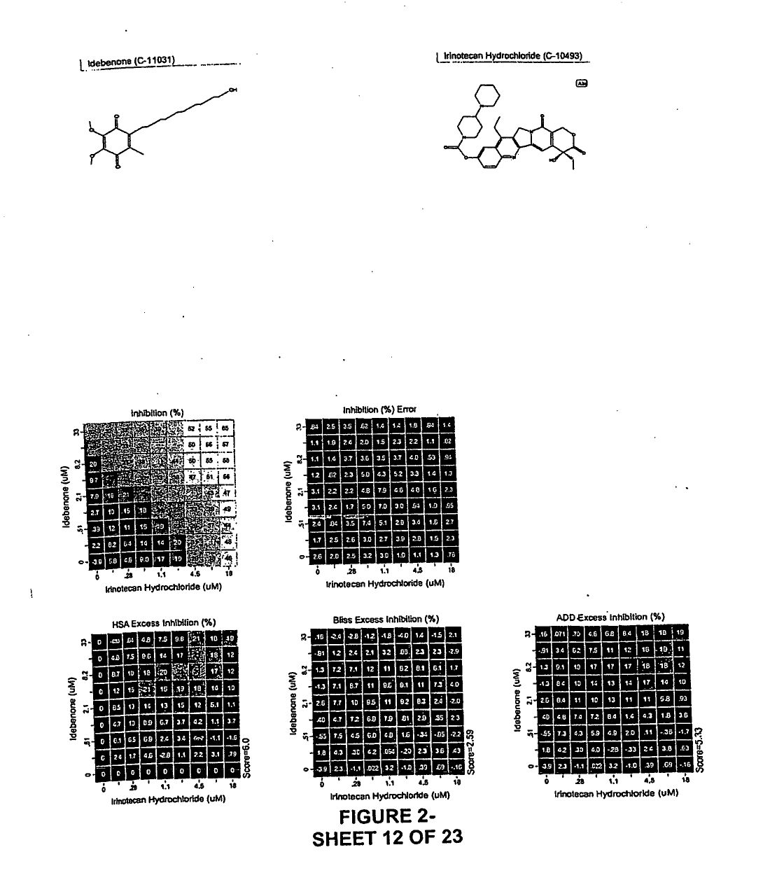

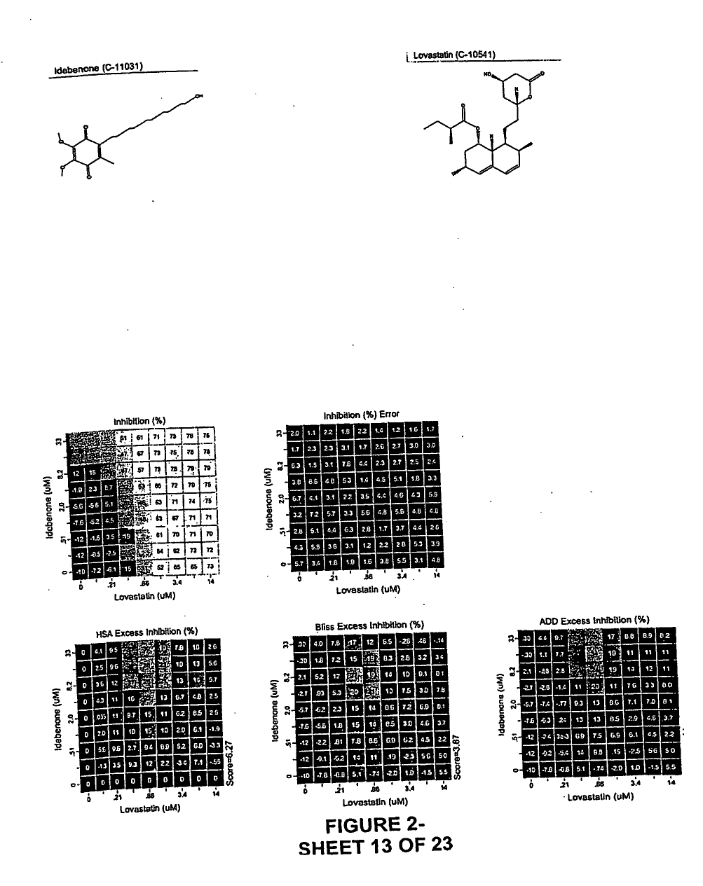

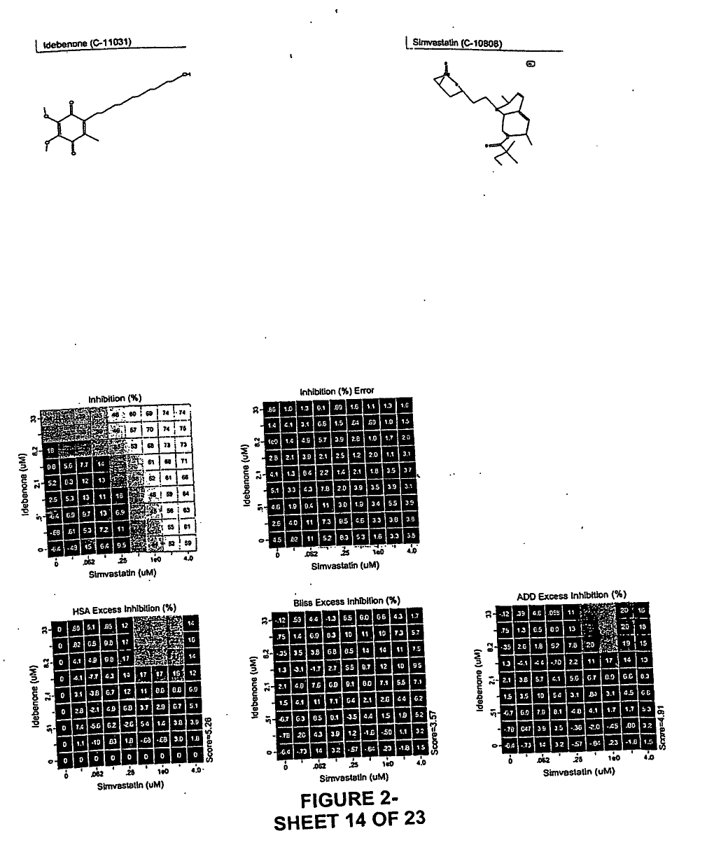

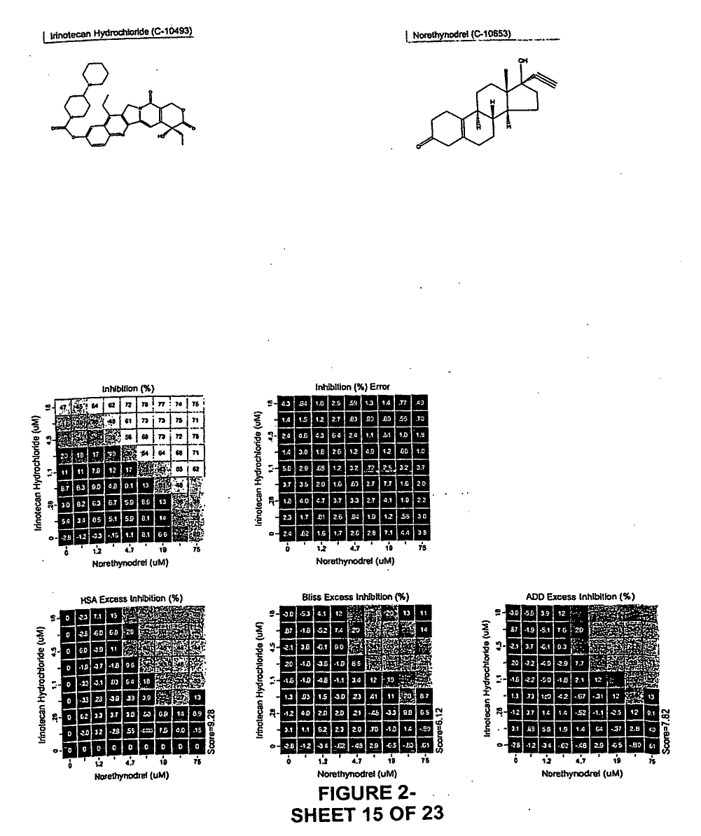

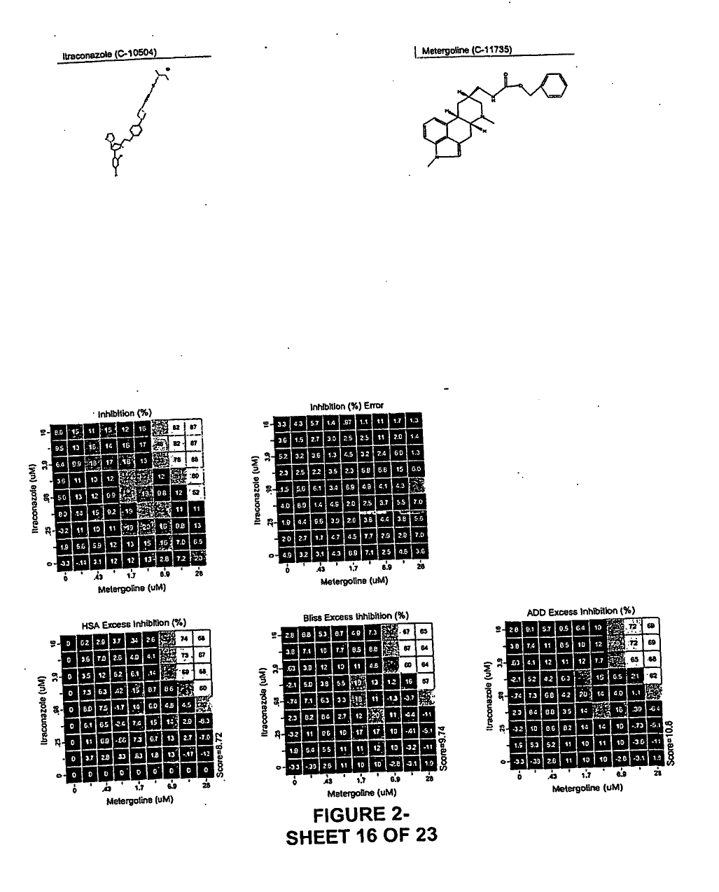

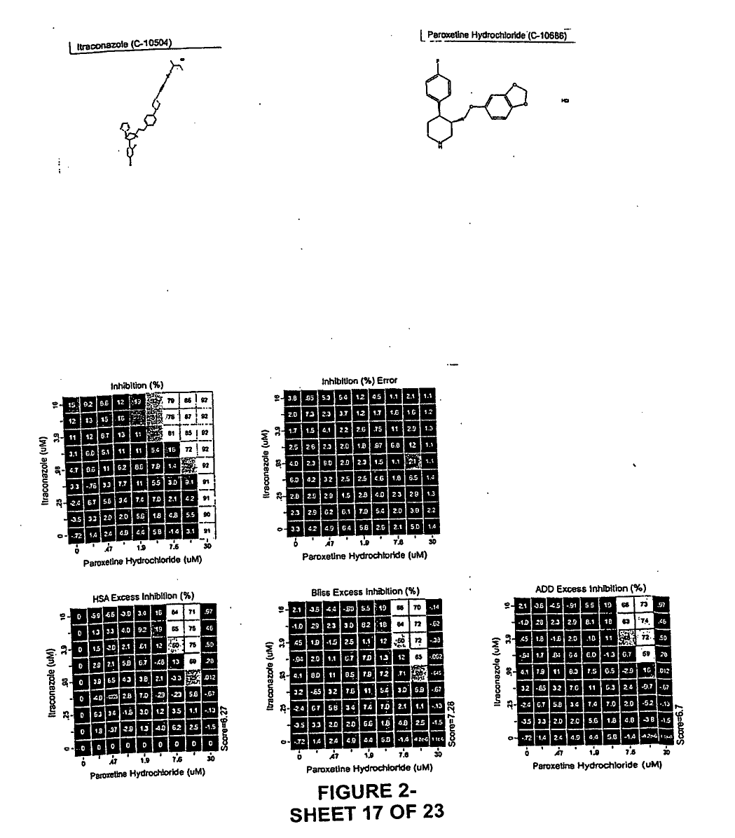

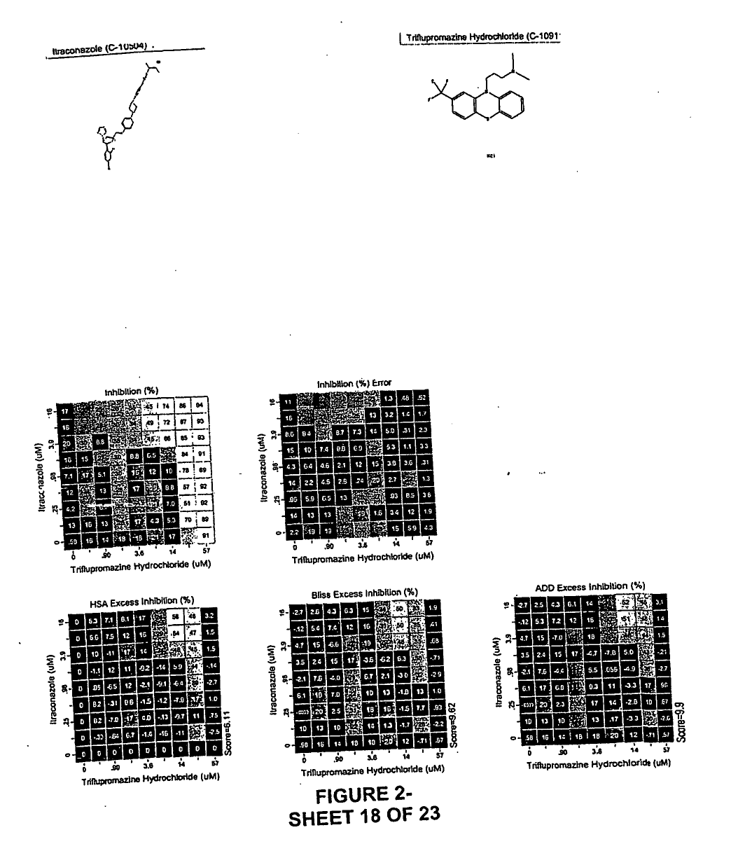

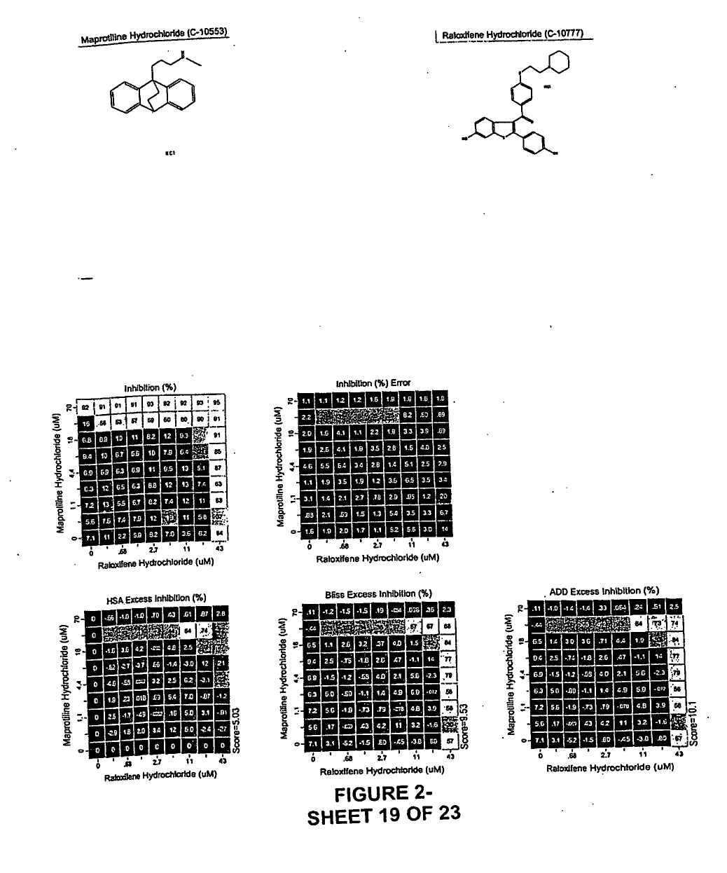

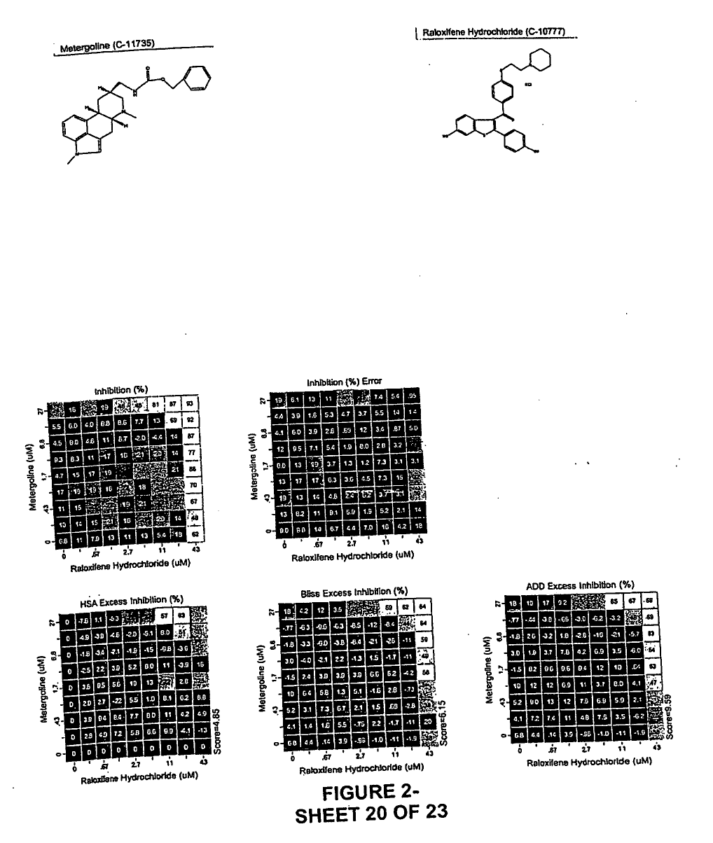

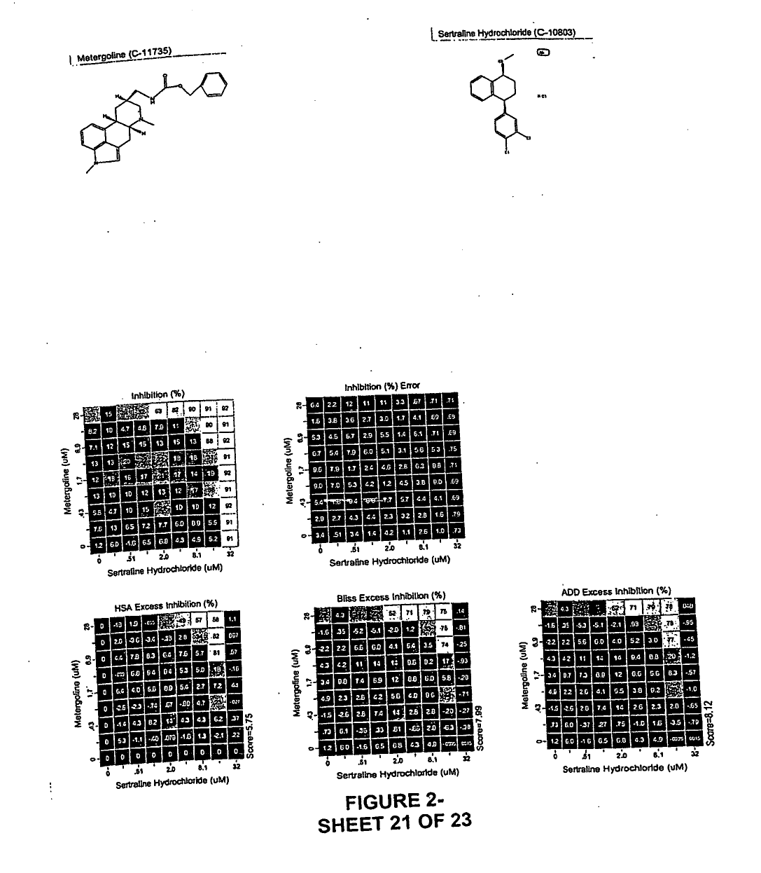

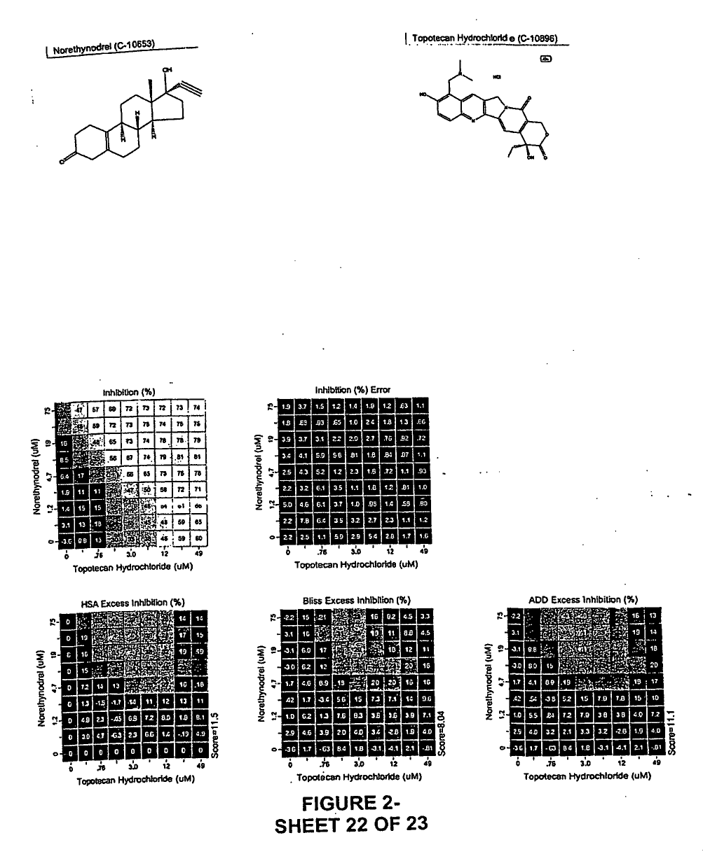

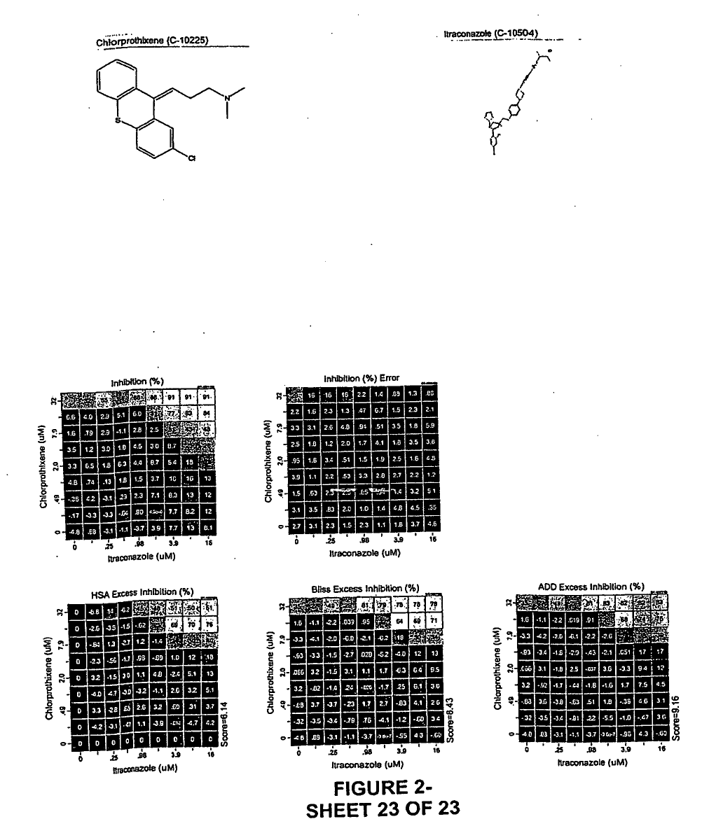

Ninety-six compounds listed in Table 1 and Table 2 (FIG. 1) were screened in all possible pairwise combinations to identify combinations exhibiting enhanced growth suppression of human D54MG cell line using the above-described anti-proliferation assay. Substantial increases in anti-proliferative activity were observed with 22 combinations (FIG. 2).

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, oncology, or related fields are intended to be within the scope of the invention.

What is claimed is:

1. A composition comprising:
(a) a first agent selected from the agents of Table 1 and Table 2; and
(b) a second agent selected from the agents of Table 1 and Table 2 other than said first agent.

2. The composition of claim 1, wherein said first agent and said second agent are present in amounts that, when administered to a patient, are effective to treat said patient.

3. The composition of claim 1, further comprising one or more additional agents selected from Table 1 or Table 2.

4. The composition of claim 1, wherein said composition is formulated for oral administration.

5. The composition of claim 1, wherein said composition is formulated for systemic administration.

6. The composition of claim 1, wherein said composition is formulated for intracranial or intrathecal administration.

7. The composition of claim 1, wherein said first agent and said second agent are selected from the group consisting of cerivastatin and adefovir dipivoxil; irinotecan and adefovir dipivoxil; lovastatin and adefovir dipivoxil; topotecan and adefovir dipivoxil; disulfiram and auranofin; cerivastatin and candesartan cilexetil; lovastatin and candesartan cilexetil; triflupromazine and carvedilol; efavirenz and cerivastatin; lovastatin and efavirenz; lovastatin and epirubicin; irinotecan and idebenone; lovastatin and idebenone; simvastatin and idebenone; norethynodrel and irinotecan; metergoline and itraconazole; paroxetine and itraconazole; triflupromazine and itraconazole; raloxifene and maprotiline; raloxifene and metergoline; sertraline and metergoline; topotecan and norethynodrel; and itraconazole and chlorprothixene.

8. A method for treating a patient having a neoplasm, said method comprising administering to said patient an agent selected from the agents of Table 1 in an amount effective to treat said patient.

9. A method for treating a patient having a neoplasm, said method comprising administering to said patient a plurality of agents each selected from any of the agents of Table 1 and Table 2, wherein said agents are administered within 28 days of each other in amounts that together are effective to treat said patient.

10. The method of claim 9, wherein said plurality of agents are cerivastatin and adefovir dipivoxil; irinotecan and adefovir dipivoxil; lovastatin and adefovir dipivoxil; topotecan and adefovir dipivoxil; disulfiram and auranofin; cerivastatin and candesartan cilexetil; lovastatin and candesartan cilexetil; triflupromazine and carvedilol; efavirenz and cerivastatin; lovastatin and efavirenz; lovastatin and epirubicin; irinotecan and idebenone; lovastatin and idebenone; simvastatin and idebenone; norethynodrel and irinotecan; metergoline and itraconazole; paroxetine and itraconazole; triflupromazine and itraconazole; raloxifene and maprotiline; raloxifene and metergoline; sertraline and metergoline; topotecan and norethynodrel; or itraconazole and chlorprothixene.

11. The method of claim 9, wherein said agents are administered within ten days of each other.

12. The method of claim 11, wherein said agents are administered within five days of each other.

13. The method of claim 12, wherein said agents are administered within twenty-four hours of each other.

14. The method of claim 8 or 9, wherein said neoplasm is cancer.

15. The method of claim 14, wherein said cancer is selected from the group consisting of brain cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, retinoblastoma, lung cancer, squamous cell carcinoma, adenocarcinoma, large cell carcinoma, and colon cancer.

16. The method of claim 15, wherein said cancer is brain cancer.

17. The method of claim 16, wherein said brain cancer is selected from the group consisting of glioblastoma, astrocytoma, glioma, meduloblastoma, oligodendroma, neuroglioma, ependymoma, and meningioma.

18. The method of claim 8 or 9, wherein said method is performed in conjunction with administering to said patient an additional treatment for a neoplasm, wherein said method and said additional treatment are administered within 6 months of each other.

19. The method of claim 18, wherein said additional treatment is administered and said method is performed within fourteen days of each other.

20. The method of claim 18, wherein said additional treatment is administered and said method is performed within five days of each other.

21. The method of claim 18, wherein said additional treatment is administered and said method is performed within twenty-four hours of each other.

22. The method of claim 18, said additional treatment comprising surgery, radiation therapy, chemotherapy, immunotherapy, anti-angiogenesis therapy, or gene therapy.

23. The method of claim 22, said additional treatment comprising chemotherapy with one or more Group A antiproliferative agents.

24. The method of claim 23, wherein said antiproliferative agent is selected from the group consisting of: bleomycin, cisplatin, daunorubicin, etoposide, melphalan, mercaptopurine, methotrexate, mitomycin, vinblastine, paclitaxel, docetaxel, vincristine, cyclophosphamide, chlorambucil, capecitabine, fludarabine, raltitrexed, doxorubicin, letrozole, anastrazole, formestane, tamoxifen, toremofine, gosereliri, leuporelin, bicalutamide, flutamide, nilutamide, hypericin, trastuzumab, and rituximab, or any combination thereof.

25. The method of claim 8 or 9, wherein said agents are administered to said patient by intravenous, intramuscular, inhalation, rectal, or oral administration.

26. The method of claim 8 or 9, wherein said agents are administered to said patient by intracranial or intrathecal administration.

27. The method of claim 8 or 9, wherein said administration further comprises administration of a compound that increases blood-brain barrier permeability.

28. The method of claim 27, wherein said compound is selected from the group consisting of a Na⁺/Ca⁺⁺ exchange blocker, mannitol, and Cereport.

29. A kit comprising:
(a) an agent selected from any one of the agents of Table 1; and
(b) instructions for administering said agent to a patient having or at risk of having a neoplasm.

30. A kit comprising:
(a) a composition comprising two agents selected from any one of the agents of Table 1 and Table 2; and
(b) instructions for administering said composition to a patient having or at risk of having a neoplasm.

31. A kit comprising:
(a) a first agent selected from any one of the agents of Table 1 and Table 2;
(b) a second agent selected from any one of the agents of Table 1 and Table 2; and
(c) instructions for administering said first and said second agents to a patient having or at risk of having a neoplasm.

32. A kit comprising:
(a) an agent selected from any one of the agents of Table 1 and Table 2; and
(b) instructions for administering said agent with a second agent selected from any one of the agents of Table 1 and Table 2 to a patient having or at risk of having a neoplasm, wherein said second agent is not the agent in (a).

33. A kit comprising:
(a) a composition comprising:
(i) a first agent selected from any one of the agents of Table 1 and Table 2; and
(ii) one or more Group A antiproliferative agents; and
(b) instructions for administering said composition to a patient having or at risk of having a neoplasm.

34. A kit comprising:
(a) a first agent selected from any one of the agents of Table 1 and Table 2;
(b) one or more Group A antiproliferative agents; and
(c) instructions for administering (a) and (b) to a patient having or at risk of having a neoplasm.

35. A kit comprising:
(a) an agent selected from any one of the agents of Table 1; and
(b) instructions for administering said agent and one or more Group A antiproliferative agents to a patient having or at risk of having a neoplasm.

36. A kit comprising:
(a) one or more Group A antiproliferative agents; and
(b) instructions for administering said agent from (a) with any agent selected from any one of the agents of Table 1 and Table 2 to a patient having or at risk of having a neoplasm.

37. A method of identifying a combination that may be useful for the treatment of a patient having a neoplasm, or the prevention or reduction of said neoplasm, said method comprising the steps of:
(a) contacting neoplastic cells with an agent selected from any one the agents of Table 1 and Table 2 and a candidate compound; and
(b) determining whether the combination of said agent and said candidate compound inhibits the growth of a neoplasm relative to cells contacted with said agent but not contacted with the candidate compound, wherein a reduction in proliferation identifies the combination as a combination useful for the treatment of a patient having a neoplasm, or the prevention or reduction of a neoplasm.

38. The method of claim 37, wherein reduction in proliferation is the result of a decreased rate of cellular division, toxicity to rapidly dividing cells, an increase in apoptotic death, or an increase in necrotic death.

39. The method of claim 37, wherein said cells are mammalian cells.

40. The method of claim 39, wherein said cells are human cells.

41. The method of claim 40, wherein said cells are selected from the group consisting of neuronal cells, glial cells, microglial cells, oligodendrocytes, and astrocytes.
